(19) United States
(12) Patent Application Publication
(10) Pub. No.: US 2025/0269069 A1
(43) Pub. Date: Aug. 28, 2025

(54) METHODS OF USING SELECTIVE DELIVERY MOLECULES

(71) Applicant: AVELAS ACQUISITION CORPORATION, La Jolla, CA (US)

(72) Inventors: Jesus E. Gonzalez, La Jolla, CA (US); Steven Chen, San Diego, CA (US); Alec Harootunian, San Diego, CA (US); Phillip Poonka, San Diego, CA (US)

(21) Appl. No.: 18/248,839

(22) PCT Filed: Oct. 8, 2021
(86) PCT No.: PCT/US2021/054313

§ 371 Date: Apr. 12, 2023

Related U.S. Application Data
(60) Provisional application No. 63/091,180, filed on Oct. 13, 2020.

(51) Int. Cl. A61K 49/00; C07K 7/08

(52) U.S. Cl. CPC A61K 49/0032; A61K 49/0056; C07K 7/08

(57) ABSTRACT

Provided herein, in part, are selective delivery molecules their methods of use in the detection of cancer tissue and surgical excision of said cancer tissue.

### CROSS-REFERENCE

This application claims the benefit of U.S. Provisional Application No. 63/091,180, filed Oct. 13, 2020, which is incorporated herein by reference in its entirety.

### BACKGROUND

Initial management of a breast cancer diagnosis usually involves surgery, carried out with the goals of both removing the tumor and staging the disease, via breast and axillary procedures, respectively. The breast surgery may consist of a lumpectomy (partial mastectomy) or a complete mastectomy. Lumpectomy is considered to be a “breast conserving surgery” procedure associated with better cosmesis and represents the majority of procedures performed presently.

There is a need for new methods for identifying cancer tumor margins intraoperatively with improved examination timelines and reduced sampling errors.

### SUMMARY

Described herein, in certain embodiments, are methods of surgically excising, evaluating, and/or imaging tissue, such as tissue comprising a plurality of cancerous cells, using the selective delivery molecule (SDM) represented by a compound of Formula I.

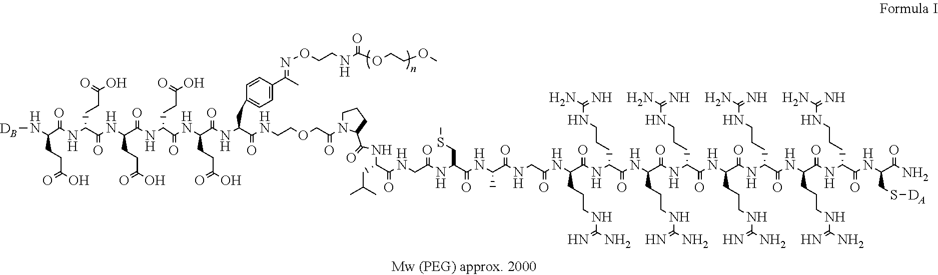

wherein: D_{A} and D_{B} are, respectively, a donor moiety and acceptor moiety that are capable of participating in Förster Resonance Energy Transfer; or a pharmaceutically acceptable salt thereof. In some embodiments, the SDM is SDM-25 as described herein.

Described herein, in certain embodiments, is a method of surgically excising a plurality of cancerous cells from a subject in need thereof, comprising: (i) administering to the subject an effective amount of a compound of Formula I,

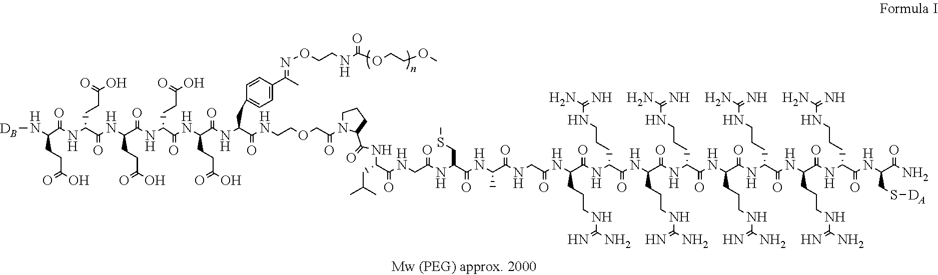

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Also disclosed herein, in certain embodiments, is a method of surgically excising a plurality of cancerous cells from a subject in need thereof, comprising: (i) administering to the subject an effective amount of a compound of Formula I,

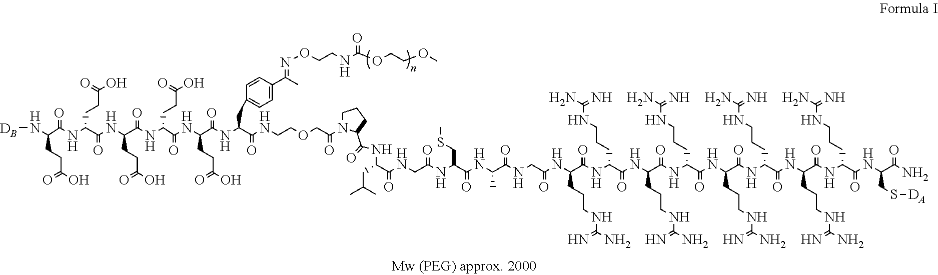

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, prior to surgery, the additional tissue excised from the surgical cavity contacted the margin of the tissue comprising the plurality of cancerous cells. In some embodiments, the method comprises surgically excising the additional tissue across the full perimeter of the surgical cavity.

Additionally disclosed herein, in certain embodiments, is a method of evaluating a tissue suspected of having a plurality of cancerous cells, comprising: (i) administering to the subject an effective amount of a compound of Formula I,

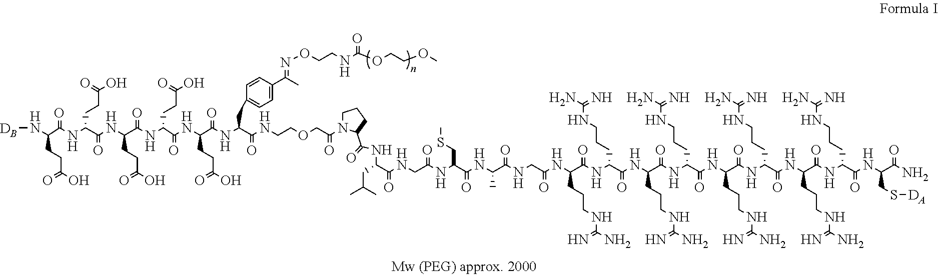

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) contacting the tissue with light having at least one excitation wavelength of the D_{A} donor moiety in Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the tissue is intact in the subject. In some embodiments, the tissue has been surgically excised from the subject.

Additionally disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of a compound of Formula I:

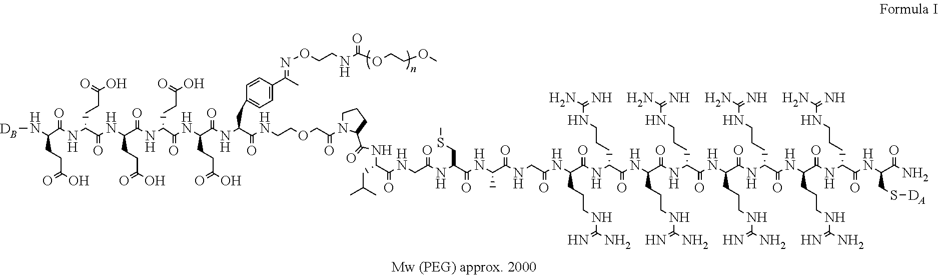

wherein: D_{A} and D_{B} are, respectively, a donor moiety and acceptor moiety that are capable of participating in Förster Resonance Energy Transfer; or a pharmaceutically acceptable salt thereof at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells.

Disclosed herein, in certain embodiments, is a method of surgically excising a plurality of cancerous cells from a subject in need thereof, comprising: (i) administering to the subject an effective amount of SDM-25,

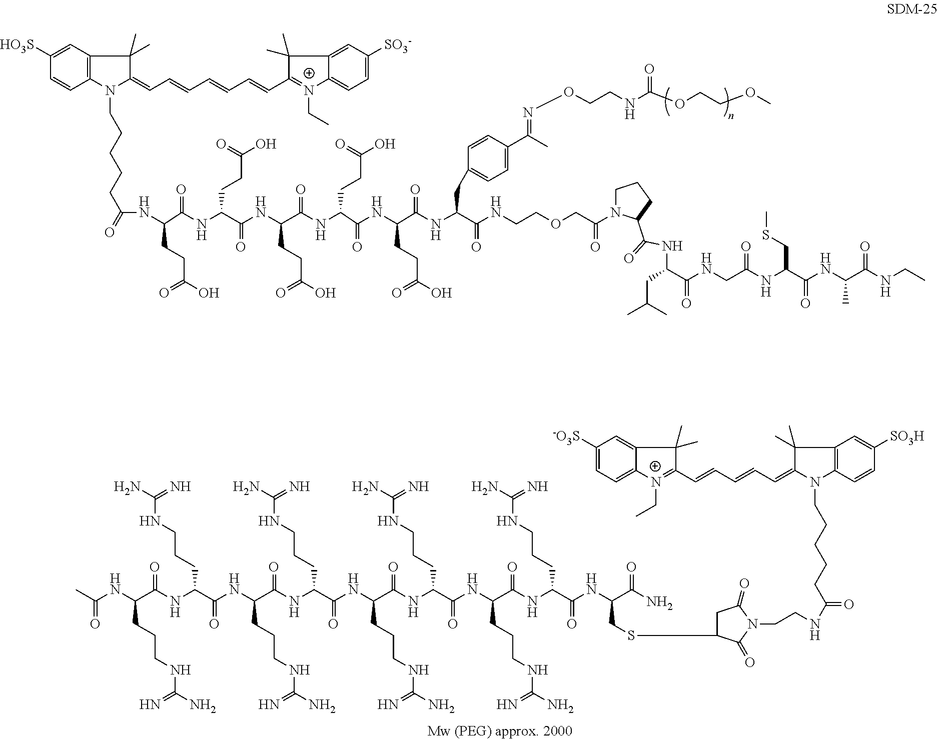

or a pharmaceutically acceptable salt thereof, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Also disclosed herein, in certain embodiments, is a method of surgically excising a plurality of cancerous cells from a subject in need thereof, comprising: (i) administering to the subject an effective amount of SDM-25,

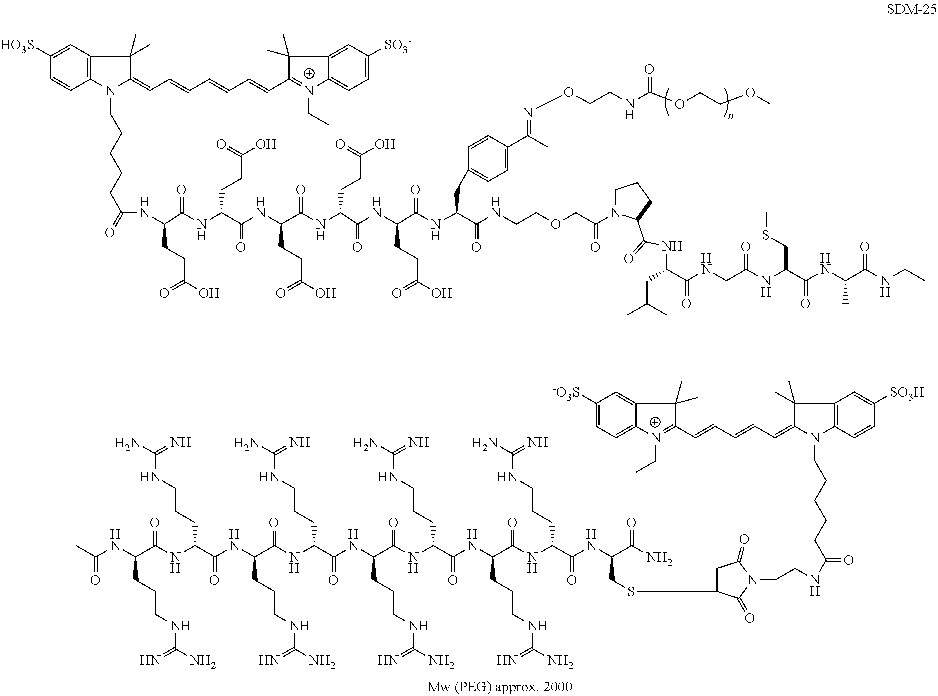

or a pharmaceutically acceptable salt thereof, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, prior to surgery, the additional tissue excised from the surgical cavity contacted the margin of the tissue comprising the plurality of cancerous cells. In some embodiments, the method comprises surgically excising the additional tissue across the full perimeter of the surgical cavity.

Additionally disclosed herein, in certain embodiments, is a method of evaluating a tissue suspected of having a plurality of cancerous cells, comprising: (i) administering to the subject an effective amount of SDM-25,

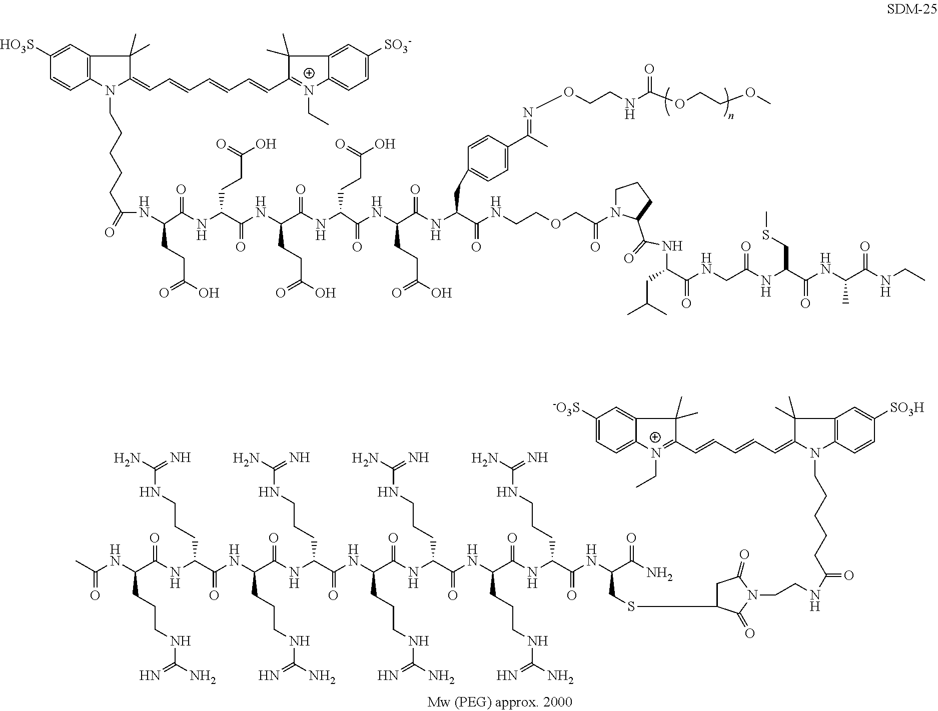

or a pharmaceutically acceptable salt thereof, at least 12 hours prior to surgery; (ii) contacting the tissue with light having at least one excitation wavelength of the Cy5 dye moiety in SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the tissue is intact in the subject. In some embodiments, the tissue has been surgically excised from the subject.

Additionally disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of SDM-25:

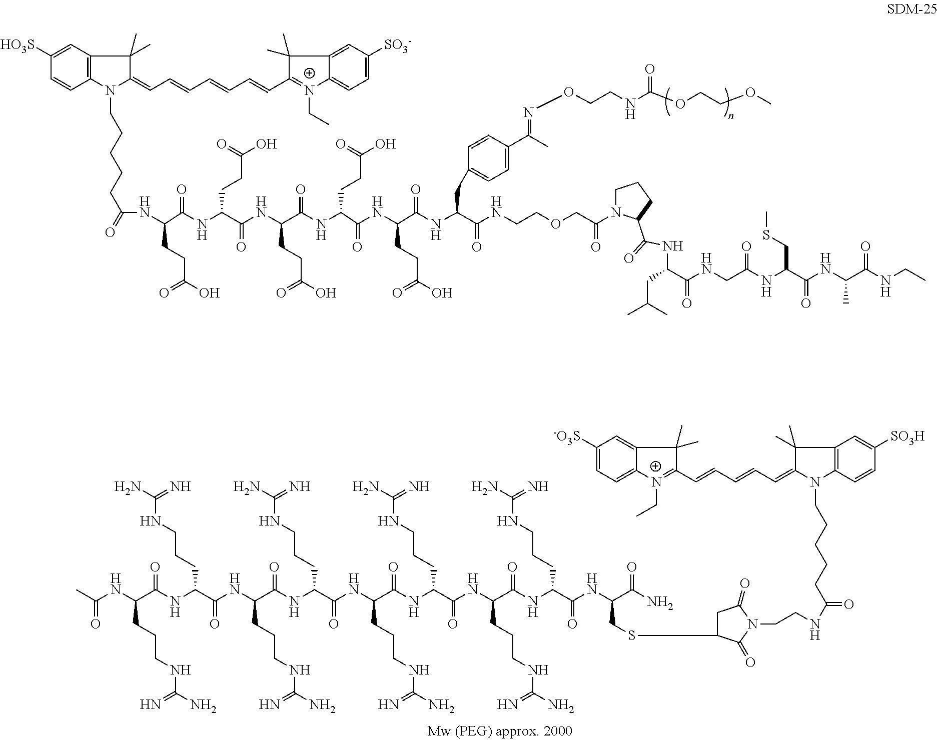

or a pharmaceutically acceptable salt thereof at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells.

Described herein, in certain embodiments, is a pharmaceutical composition configured in single dose form, wherein the single dose form comprises 1 mg to 16 mg of a compound of Formula I:

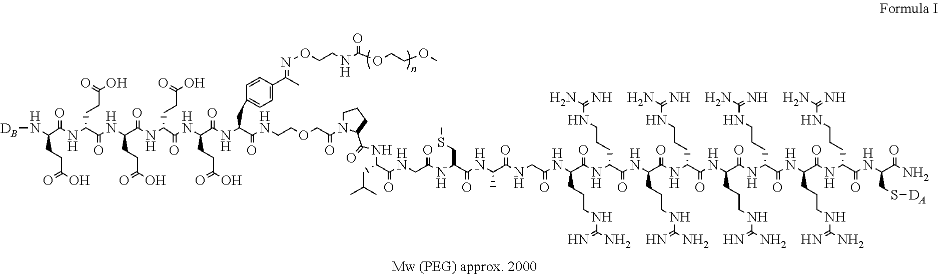

wherein: D_{A} and D_{B} are, respectively, a donor moiety and acceptor moiety that are capable of participating in Förster Resonance Energy Transfer; or a pharmaceutically acceptable salt thereof.

Described herein, in certain embodiments, is a pharmaceutical composition configured in single dose form, wherein the single dose form comprises 1 mg to 16 mg of a compound of Formula I:

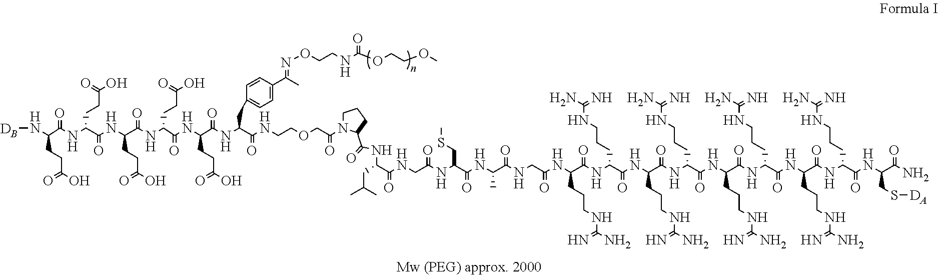

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Described herein, in certain embodiments, is a pharmaceutical composition configured in single dose form, wherein the single dose form comprises 1 mg to 16 mg of a compound of Formula I:

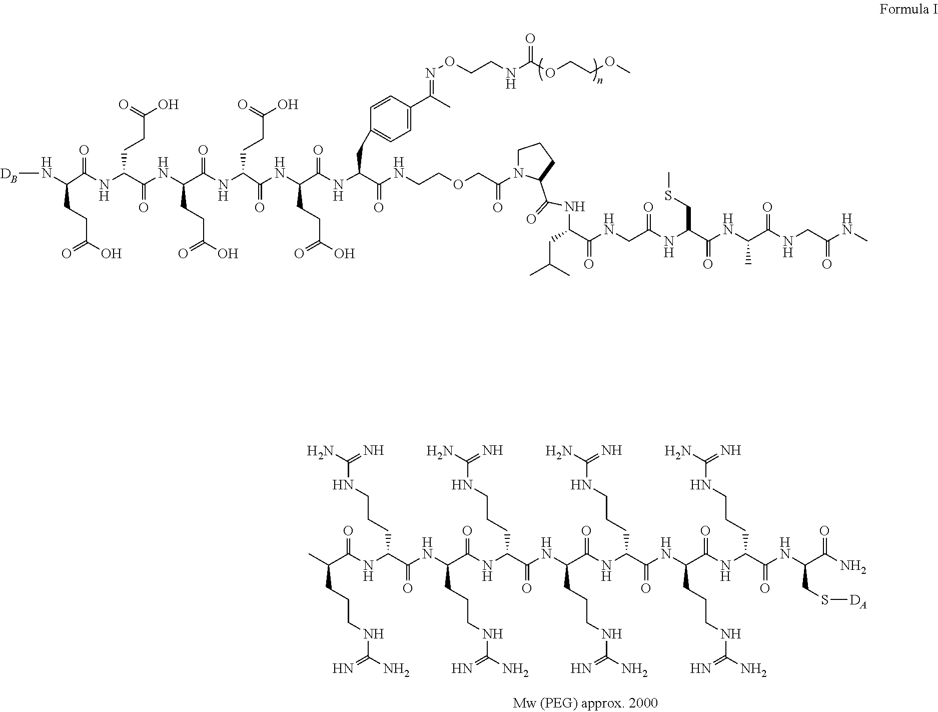

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition; at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, prior to surgery, the additional tissue excised from the surgical cavity contacted the margin of the tissue comprising the plurality of cancerous cells. In some embodiments, the method comprises surgically excising the additional tissue across the full perimeter of the surgical cavity.

Described herein, in certain embodiments, is a pharmaceutical composition configured in single dose form, wherein the single dose form comprises 1 mg to 16 mg of a compound of Formula I:

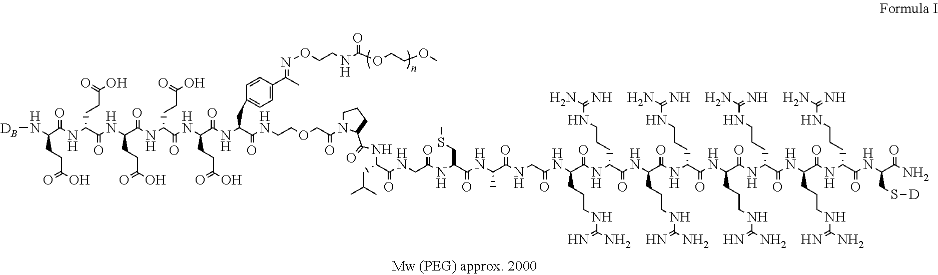

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the D_{A} donor moiety in the compound of Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the tissue is intact in the subject. In some embodiments, the tissue has been surgically excised from the subject.

Described herein, in certain embodiments, is a pharmaceutical composition configured in single dose form, wherein the single dose form comprises 1 mg to 16 mg of a compound of Formula I:

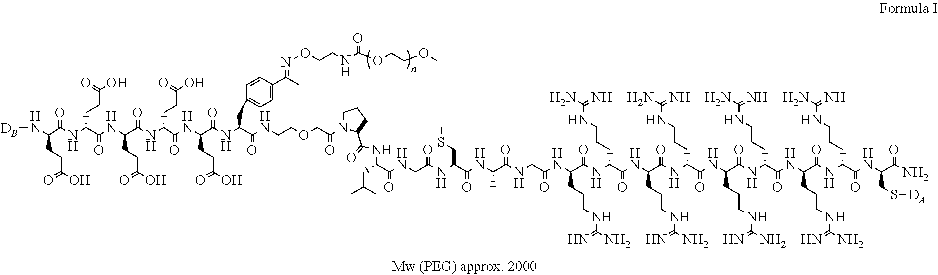

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in a single dose form, wherein the single dose form comprises 1 mg to 16 mg of a compound of SDM-25:

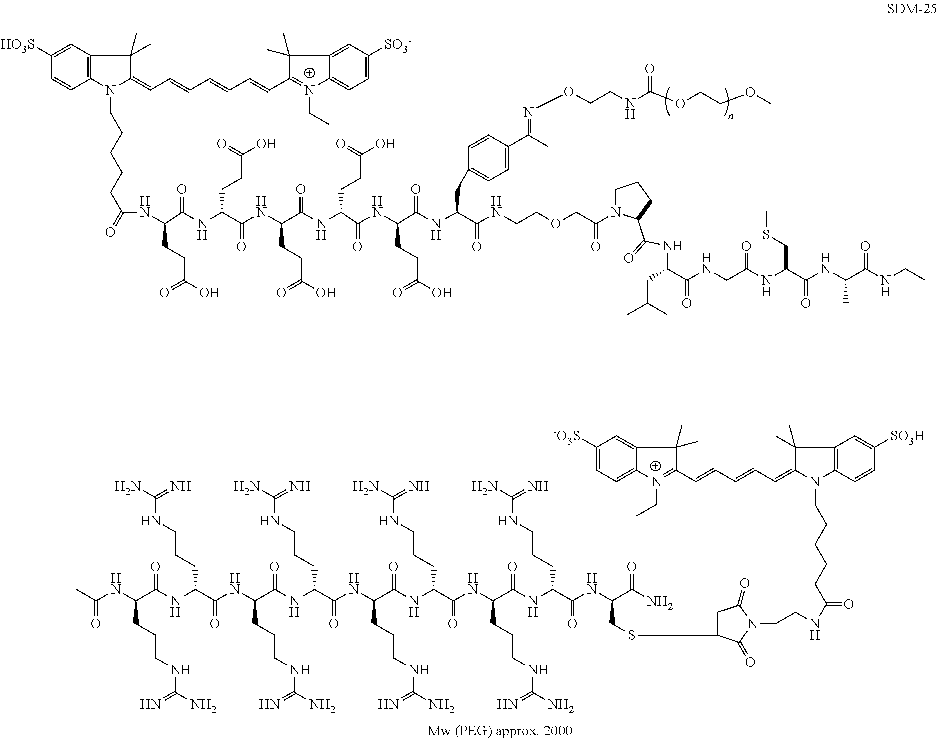

or a pharmaceutically acceptable salt thereof.

Described herein, in certain embodiments, is a pharmaceutical composition configured in single dose form, wherein the single dose form comprises 1 mg to 16 mg of SDM-25:

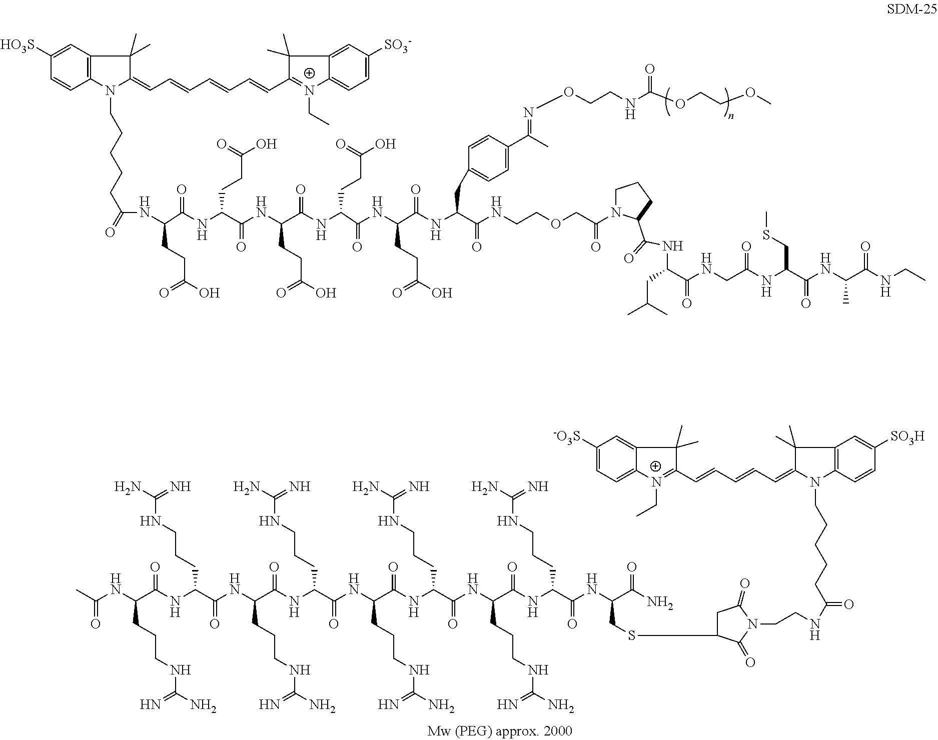

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Described herein, in certain embodiments, is a pharmaceutical composition configured in single dose form, wherein the single dose form comprises 1 mg to 16 mg of SDM-25:

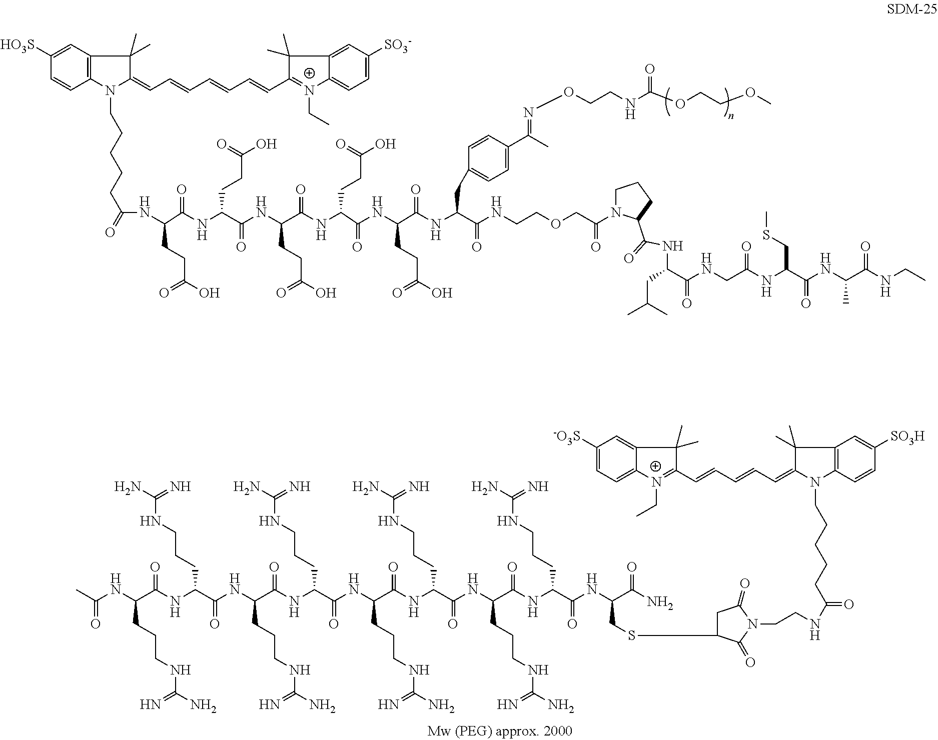

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, prior to surgery, the additional tissue excised from the surgical cavity contacted the margin of the tissue comprising the plurality of cancerous cells. In some embodiments, the use comprises surgically excising the additional tissue across the full perimeter of the surgical cavity.

Described herein, in certain embodiments, is a pharmaceutical composition configured in single dose form, wherein the single dose form comprises 1 mg to 16 mg of SDM-25:

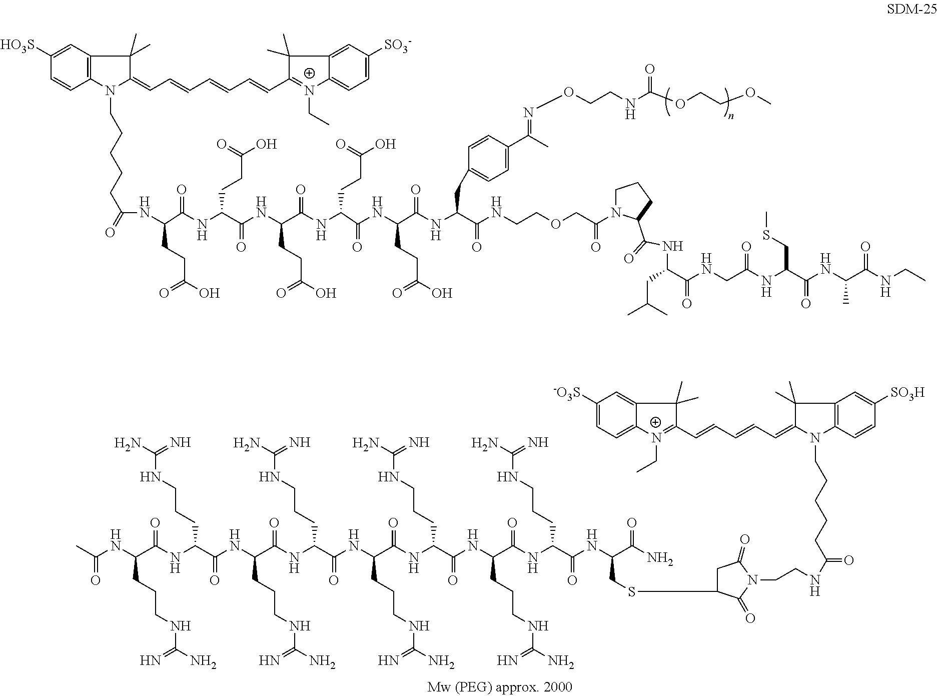

or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the Cy5 dye moiety of SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the tissue is intact in the subject. In some embodiments, the tissue has been surgically excised from the subject.

Described herein, in certain embodiments, is a pharmaceutical composition configured in single dose form, wherein the single dose form comprises 1 mg to 16 mg of SDM-25:

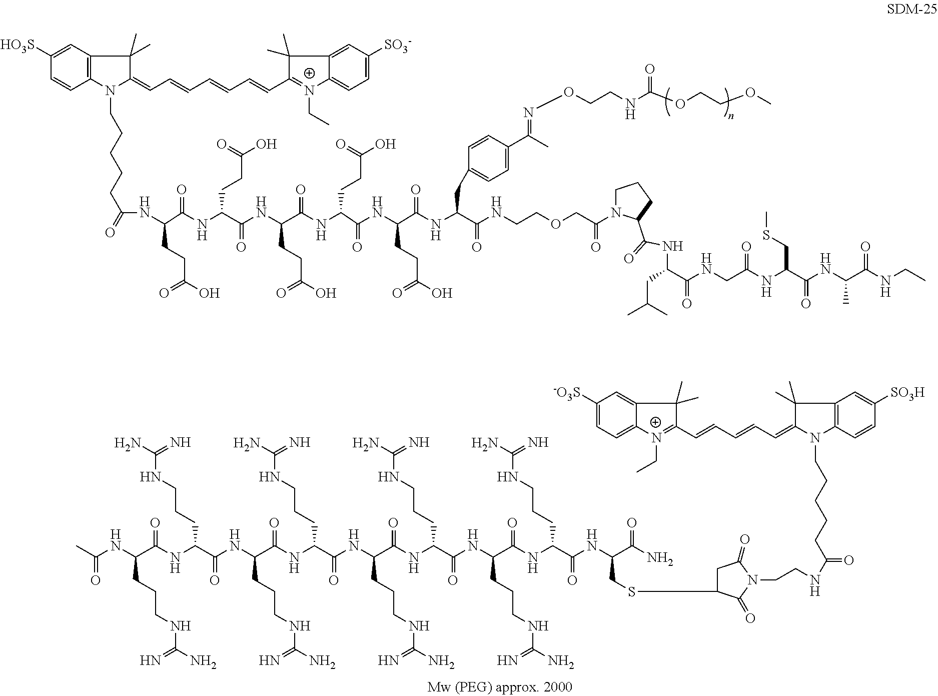

or a pharmaceutically acceptable salt thereof, for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells.

### BRIEF DESCRIPTION OF THE DRAWINGS

FIG. 1 depicts an exemplary schematic of SDM-25 Mechanism: (Left) SDM-25 is an activatable cell penetrating peptide (ACPP). A masking sequence (red) neutralizes the cell penetrating peptide (CPP) when tethered by protease cleavable linker. In the intact structure, illumination with light of wavelengths able to photoexcite Cy5 dye attached to CPP results in efficient Förster Resonance Energy Transfer (FRET) from Cy5 to Cy7 attached to end of mask sequence, due to close proximity and overlap of Cy5 emission spectrum with Cy7 excitation spectrum. FRET result in highly quenched Cy5 and sensitized emission from Cy7. (Right) SDM-25 is a substrate to proteases in the MMP family which have higher expression in cancerous tissue compared to non-malignant tissue. Cleavage at the substrate site results in mask to dissociate from CPP which activates the CPP and disrupts FRET. This results in a dramatic change in fluorescence color from Cy7 to Cy5 and with great increase in Cy5 intensity. The unmasked CPP also enhances tissue retention of Cy5 for a long-lasting fluorescence signal.

FIG. 2 depicts representative images of SDM-25 mediated visualization of cancerous tissue from human breast cancer patients. (Left) Excised primary lumpectomy specimen images with lightly-shaded overlay regions (encompassed by a circle) indicating fluorescence above pre-specified fluorescence ratio and intensity thresholds. The same specimen surfaces were confirmed by histopathology to contain cancer. (Right) In vivo cavity (surgical field after lumpectomy specimen removed) shaves with histopathology confirmed cancer (also known as positive margin). A lightly-shaded overlay encompassed by a circle shows locations with fluorescence above pre-specified thresholds.

FIG. 3 depicts an exemplary patient-level analysis of margin relevant tissues (lumpectomy surfaces and cavity shaves): The percentage of SDM-25 administered patients with intraoperative positive margins detected by fluorescence imaging is described as sensitivity (TP/(TP+FN)). The patient-level sensitivity is shown (black bars) for same day of surgery (SDS) and day before surgery (DBS) dosing groups. The DBS groups was significantly greater (65% versus 25%) compared to SDS groups (p value=0.005). The percentage of patients with intraoperative negative margins detected is described as specificity (TN/(TN+FP)). The patient-level specificity is shown (hatched bar). The SDS group was 60.0% and DBS group was 77.8%. The DBS group had higher detection performance for both positive and negative margins compared to SDS dosing. This data was prospectively collected using pre-specified thresholds and considered close margin as negative.

FIG. 4 depicts an exemplary patient-level analysis of cavity shaves: The percentage of patients with intraoperative positive margins on cavity shaves is described as sensitivity (TP/(TP+FN)). The patient-level sensitivity is shown (black bars) for SDS and DBS dosing groups. The DBS groups was significantly greater (71.4% versus 14.3%) compared to SDS groups (p value=0.003). The percentage of patients with intraoperative negative margins detected is described as specificity (TN/(TN+FP)). The patient-level specificity is shown (hatched bar). The SDS group was 88.5% and DBS group was 81.1%. The DBS group had superior detection performance compared to SDS dosing. This data was prospectively collected using pre-specified thresholds.

FIG. 5 depicts SDM-25 imaging method intraoperatively identified up to 75% patients with a cancer within 2 mm of surface (close margin) in margin-relevant tissues (lumpectomy surfaces and cavity shaves) in patients dosed DBS. The intraoperative positive margin rate was 42.6% in patients dosed DBS as determined by pathology. This result indicates that up to 75% of patients that could otherwise be candidates for a re-excision operation may be helped and avoid a second operation.

FIG. 6 depicts an exemplary time dependence of Cy5/Cy7 fluorescence ratio in transected tumor (diamonds) and non-malignant adjacent breast tissue (triangles). Time (hours) is interval from end of SDM-25 infusion administration and start of surgery. Data show that ratio increases with time and that a minimum amount of time is needed to generate sufficient and maximal ratio difference between malignant and non-malignant tissue. DBS dosing is typically ≥12 hours.

FIG. 7 depicts a flowchart describing the SDM-25 imaging procedure during a lumpectomy in breast cancer patients. Lumpectomy is defined as the surgical removal of a breast malignancy with the intention to achieve negative margins, while not removing the entire breast.

FIG. 8 depicts a flowchart describing where the imaging read values and pathology read values are generated and where they are combined to generate the imaging-pathology correlation value. The logic described in Table 2 as described herein is programmatically implemented from data in the electronic case report form (eCRF) to output the imaging-pathology correlation value based on the imaging read value and pathology read value.

### DETAILED DESCRIPTION

The goal of tumor resection is to remove all of the cancerous tissue to prevent recurrence and associated negative outcomes including metastasis and death. However, cancerous tissue is often difficult to distinguish from non-cancerous tissue based solely on visual inspection and touch. To determine whether cancerous tissue has been fully excised, the excised tissue is sent to a pathologist. The pathologist will identify the tumor as having positive, close or negative margins. Histopathology and margin status results often take 1-3 weeks after completion of surgery. In some embodiments, if cancerous cells are identified on the surface of the excised tumor specimen, the pathologist classifies this as a positive margin. In certain embodiments, identification of cancerous cells within 2 mm of the surface of the excised tissue will result in the tumor being classified as having positive margins. In other embodiments, identification of cancerous cells within 2 mm of the surface of the excised tissue will result in the tumor being classified as having close margins. Positive margins are associated with higher rates of local recurrence.

In the context of breast cancer surgery, the current standard of care is to excise the tumor to a negative margin. Positive tumor margins remain a challenge during breast cancer surgery. Surgeons need to rely on physical examination, tissue texture, and correlation with radiographic data to guide attempts to achieve negative margins. However, despite these tools, positive margin rates for breast-conserving surgeries are 30%-40% when prospectively measured, and re-excision rates are as high as 86%, but more typically in the 20% to 40% range. Untreated positive margins are associated with higher rates of local recurrence and thus re-excision is the standard of care whenever feasible. Approximately ⅓ of patients undergoing re-excision will choose a mastectomy in an attempt to achieve a negative margin as finding a positive margin in a second surgery can be difficult. Improved intraoperative tumor margin detection would help mitigate these serious and possibly life-threatening consequences of postsurgical positive tumor margins.

While the amount of time between surgical excision of a tumor and the pathology report varies, on average the pathology report is available 3 to 7 days after surgery. By this time, the patient has returned home and begun the healing process. Many patients are often reluctant to undergo additional surgical excision due to the significant pain, anxiety and cost associated with these surgeries. As with any surgery involving general anesthesia, there is also a risk of mortality. Further, re-excision has been associated with poorer cosmetic outcomes and higher rates of recurrence due to delays in commencing adjuvant therapy. Thus, there is a need for methods to reduce re-excision by identifying positive or close margins during surgical procedures.

The only currently available technology indicated for decreasing margin positivity is the MARGINPROBE® System, which was approved by the Food and Drug Administration (FDA) in 2012. MarginProbe provides a real-time assessment of cancer on the margin of excised human tissue, by assessing the electromagnetic response of the tissue and then comparing those responses to an internal database of known signatures in healthy and cancerous tissues. MARGINPROBE® demonstrated up to a 56% decrease in re-excision rate in one study of ductal carcinoma in situ (DCIS). However, a multicenter randomized trial reported a 6% absolute reduction in excisions from 25.8% to 19.8%, and a false positive rate of 53.6% and a false negative rate of 24.8%. In addition, multiple separate measurements per specimen may be required, as the sensor area is only 7 mm in diameter (e.g., up to 25 individual measurements per sample). This limitation makes examination of large specimens time consuming and increases the likelihood of sampling error. In addition, the required use of a probe requires touching the tissue and is not applicable to interrogating the surgical cavity.

The present disclosure will now be described more fully hereinafter with reference to the accompanying Figures and Examples, in which representative embodiments are shown. The present disclosure can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the disclosure pertains. All publications, patent applications, patents, and other references mentioned herein are incorporated by reference in their entirety.

### Definitions

As used herein, the following terms have the meanings ascribed to them unless specified otherwise.

Unless otherwise defined, all technical terms used herein have the same meaning as commonly understood by one of ordinary skill in the art in the field to which this disclosure belongs. As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “ratiometric fluorescent indicator” (or “ratiometric fluorescence probe”) and “ratiometric fluorescence imaging” are used broadly to include not only the use of conventional ratiometric indicators (e.g., Forster/fluorescence resonance energy transfer (FRET)-based probes) and associated imaging techniques but also, for example, instances where a combination of two or more fluorophores are used (e.g., fluorophores that are not capable of FRET pairing), with one fluorophore serving as an internal control and at least one other fluorophore serving as an indicator for the biological activity of interest. In some instances, a ratiometric fluorescent indicator is an imaging agent, e.g., for use in in vivo imaging applications.

As used herein, the term “excitation wavelength” refers to the wavelength of light used to excite a fluorescent indicator (e.g., a fluorophore or dye molecule) and generate fluorescence. Although the excitation wavelength is typically specified as a single wavelength, e.g., 620 nm, it will be understood by those of skill in the art that this specification refers to a wavelength range or excitation filter bandpass that is centered on the specified wavelength. In some instances, light of the specified excitation wavelength comprises light of the specified wavelength ±2 nm, ±5 nm, ±10 nm, ±20 nm, ±40 nm, ±80 nm, or more. In some instances, the excitation wavelength used may or may not coincide with the absorption peak maximum of the fluorescent indicator.

As used herein, the term “emission wavelength” refers to the wavelength of light emitted by a fluorescent indicator (e.g., a fluorophore or dye molecule) upon excitation by light of an appropriate wavelength. Although the emission wavelength is typically specified as a single wavelength, e.g., 670 nm, it will be understood by those of skill in the art that this specification refers to a wavelength range or emission filter bandpass that is centered on the specified wavelength. In some instances, light of the specified emission wavelength comprises light of the specified wavelength ±2 nm, ±0.5 nm, ±10 nm, ±20 nm, ±40 nm, ±80 nm, or more.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “administer,” “administering”, “administration,” and the like, as used herein, refer to the methods that may be used to enable delivery of agents or compositions to the desired site of biological action. These methods include, but are not limited to parenteral injection (e.g., intravenous, subcutaneous, intraperitoneal, intramuscular, intravascular, intrathecal, intravitreal, infusion, or local). Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “cancer” as used herein, refers to any disease involving uncontrolled growth or proliferation cells in the human body. Cancers may further be characterized by the ability of cells to migrate from the original site and spread to distant sites (i.e., metastasize). Cancers may be sarcomas, carcinomas, lymphomas, leukemias, blastomas, or germ cell tumors. Cancers may occur in a variety of tissues including but not limited to lung, breast, ovaries, colon, esophagus, rectum, bone, prostate, brain, pancreas, bladder, kidney, liver, blood cells, lymph nodes, and stomach.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In general, the “effective amount” of a compound refers to an amount sufficient to elicit a desired fluorescence intensity, or intensity change, of one or more fluorescent molecules in cancer tissue. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject.

The terms “Same Day Dosing” and “Same Day Surgery” are used interchangeably herein. The terms “Day Before Dosing” and “Day Before Surgery” are used interchangeably herein.

As used herein, a “fluorescence ratio image of Cy5 dye moiety fluorescence intensity to Cy7 dye moiety fluorescence intensity” refers to a fluorescence ratio image obtained by: (i) capturing a fluorescence intensity image of Cy5 dye moiety fluorescence across a region (e.g., a region of a tissue described herein), and a second fluorescence intensity image of Cy7 dye moiety fluorescence across the same region; and (ii) combining the first fluorescence intensity image and the second fluorescence intensity image to create the fluorescence ratio image.

“Specificity” at the patient level, and as used herein, refers to the percentage of a cancer patient population with intraoperative positive tumor margins (as determined by gold standard pathology), including margins on primary specimen surfaces and margins on cavity shaves, correctly identified with fluorescence imaging. It is calculated as (TP/(TP+FN)), where “TP” refers to the number of patients reported as true positives, and “FN” refers to the number of patients reported as false negatives.

“Sensitivity” at the patient level, and as used herein, refers to the percentage of a cancer patient population with intraoperative negative tumor margins, including margins on cavity shaves, which is (TN/(TN+FP)), where “TN” refers to the number of patients reported as true negatives, and “FP” refers to the number of patients reported as false positives.

Unless otherwise specified, sensitivity and specificity values described herein are reported under the close as negative (CAN) definition. The CAN definition defines cancerous tissue as being on the surface of the margin (0 mm away) for invasive cancer and up to 2 mm away from the surface of the margin for a non-invasive cancer (e.g., DCIS). In cases with mixed cancers, each type utilizes the same definition as if it was not mixed.

### Selective Delivery Molecules of Formula I

Described herein, in certain embodiments, are methods of surgically excising, evaluating, and imaging tissue, such as tissue comprising a plurality of cancerous cells, using the selective delivery molecule (SDM) represented by a compound of Formula I.

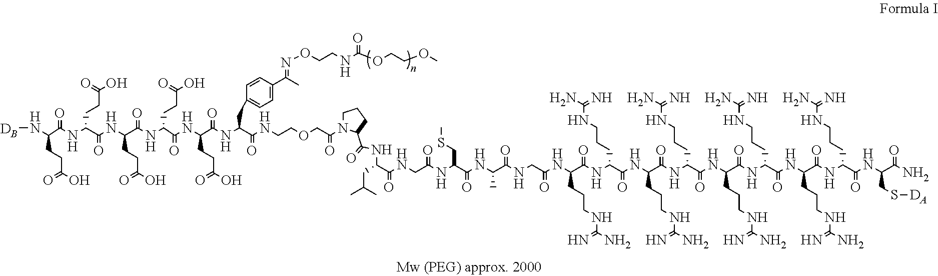

wherein: D_{A} and D_{B} are, respectively, a donor moiety and acceptor moiety that are capable of participating in Förster Resonance Energy Transfer; or a pharmaceutically acceptable salt thereof. In some embodiments, D_{A} and D_{B} are, respectively, selected from Cy5 and Cy7, Cy5 and IRDye750, Cy5 and IRDye800, and Cy5 and ICG.

Described herein, in certain embodiments, is a method of surgically excising a plurality of cancerous cells from a subject in need thereof, comprising: (i) administering to the subject an effective amount of a compound of Formula I,

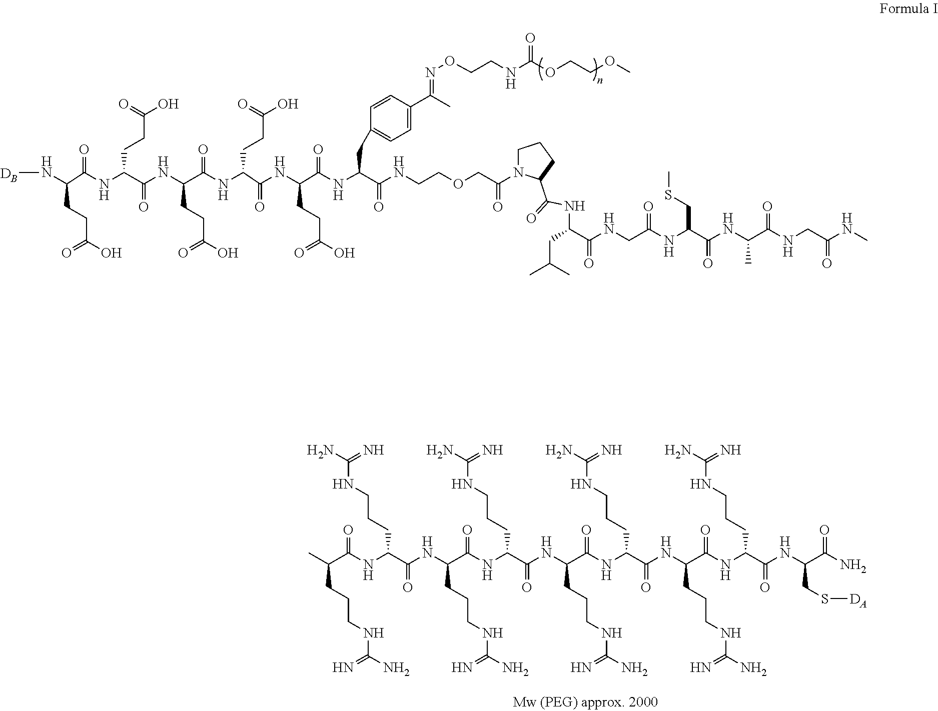

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, D_{A} and D_{B} are, respectively, selected from Cy5 and Cy7, Cy5 and IRDye750, Cy5 and IRDye800, and Cy5 and ICG.

Also disclosed herein, in certain embodiments, is a method of surgically excising a plurality of cancerous cells from a subject in need thereof, comprising: (i) administering to the subject an effective amount of a compound of Formula I,

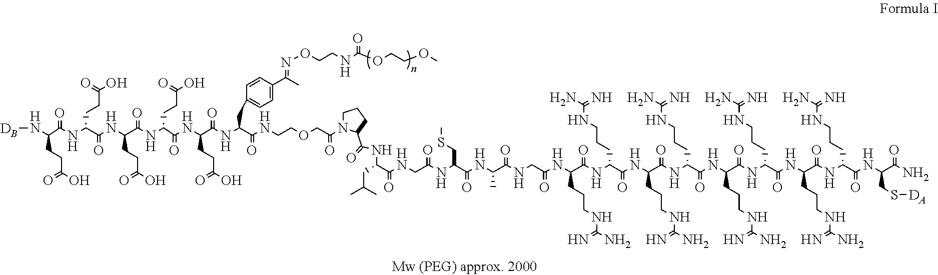

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, D_{A} and D_{B} are, respectively, selected from Cy5 and Cy7, Cy5 and IRDye750, Cy5 and IRDye800, and Cy5 and ICG.

Additionally disclosed herein, in certain embodiments, is a method of evaluating a tissue suspected of having a plurality of cancerous cells, comprising: (i) administering to the subject an effective amount of a compound of Formula I,

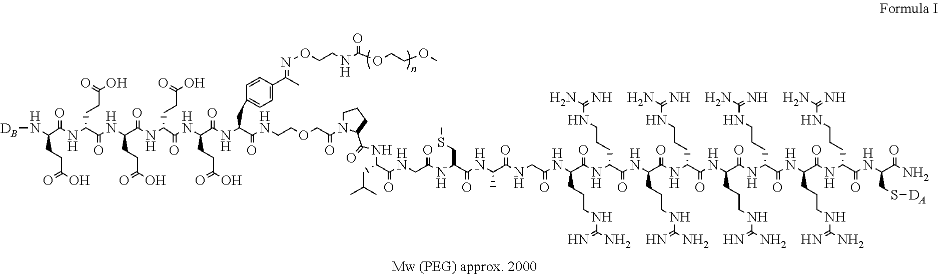

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) contacting the tissue with light having at least one excitation wavelength of the D_{A} donor moiety in Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, D_{A} and D_{B} are, respectively, selected from Cy5 and Cy7, Cy5 and IRDye750, Cy5 and IRDye800, and Cy5 and ICG. In some embodiments, the tissue is intact in the subject. In some embodiments, the tissue has been surgically excised from the subject. In some embodiments, prior to surgery, the additional tissue excised from the surgical cavity contacted the margin of the tissue comprising the plurality of cancerous cells. In some embodiments, the method comprises surgically excising the additional tissue across the full perimeter of the surgical cavity.

Additionally disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of a compound of Formula I:

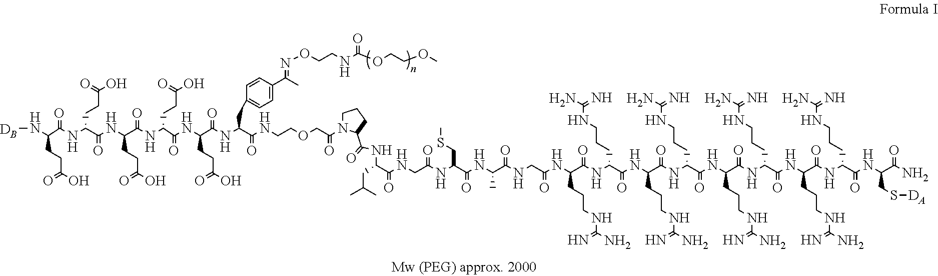

wherein: D_{A} and D_{B} are, respectively, a donor moiety and acceptor moiety that are capable of participating in Förster Resonance Energy Transfer; or a pharmaceutically acceptable salt thereof at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, D_{A} and D_{B} are, respectively, selected from Cy5 and Cy7, Cy5 and IRDye750, Cy5 and IRDye800, and Cy5 and ICG.

Pharmaceutical compositions configured as dosage forms for use in the methods described herein are also provided. Described herein, in certain embodiments, is a pharmaceutical composition configured in single dose form, wherein the single dose form comprises 1 mg to 16 mg of a compound of Formula I:

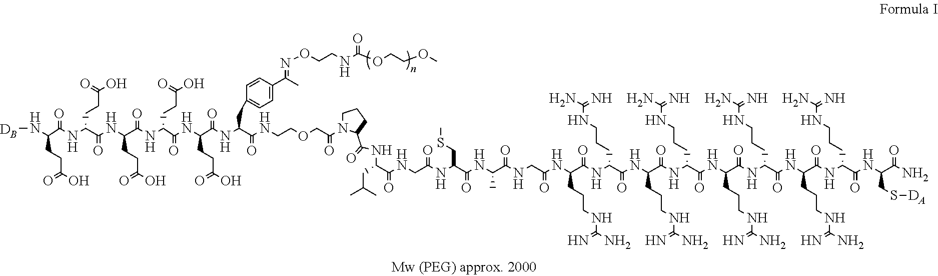

wherein: D_{A} and D_{B} are, respectively, a donor moiety and acceptor moiety that are capable of participating in Förster Resonance Energy Transfer; or a pharmaceutically acceptable salt thereof. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, D_{A} and D_{B} are, respectively, selected from Cy5 and Cy7, Cy5 and IRDye750, Cy5 and IRDye800, and Cy5 and ICG.

Described herein, in certain embodiments, is a pharmaceutical composition configured in single dose form, wherein the single dose form comprises 1 mg to 16 mg of a compound of Formula I:

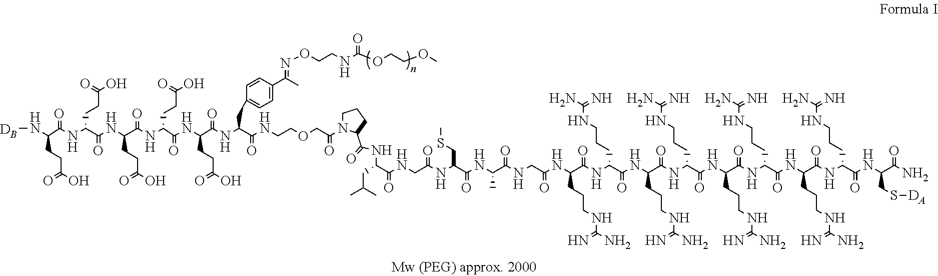

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, D_{A} and D_{B} are, respectively, selected from Cy5 and Cy7, Cy5 and IRDye750, Cy5 and IRDye800, and Cy5 and ICG.

Described herein, in certain embodiments, is a pharmaceutical composition configured in single dose form, wherein the single dose form comprises 1 mg to 16 mg of a compound of Formula I:

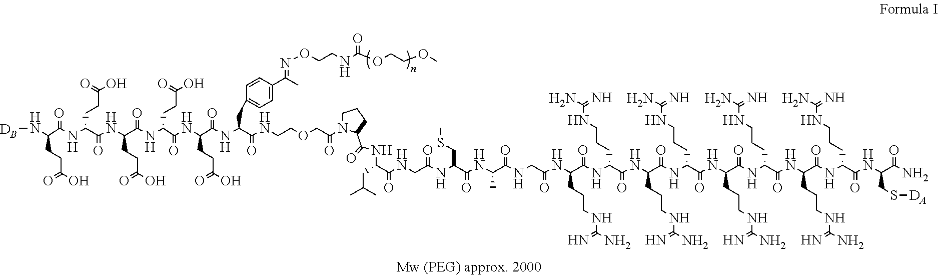

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition; at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, D_{A} and D_{B} are, respectively, selected from Cy5 and Cy7, Cy5 and IRDye750, Cy5 and IRDye800, and Cy5 and ICG. In some embodiments, prior to surgery, the additional tissue excised from the surgical cavity contacted the margin of the tissue comprising the plurality of cancerous cells. In some embodiments, the use comprises surgically excising the additional tissue across the full perimeter of the surgical cavity.

Described herein, in certain embodiments, is a pharmaceutical composition configured in single dose form, wherein the single dose form comprises 1 mg to 16 mg of a compound of Formula I:

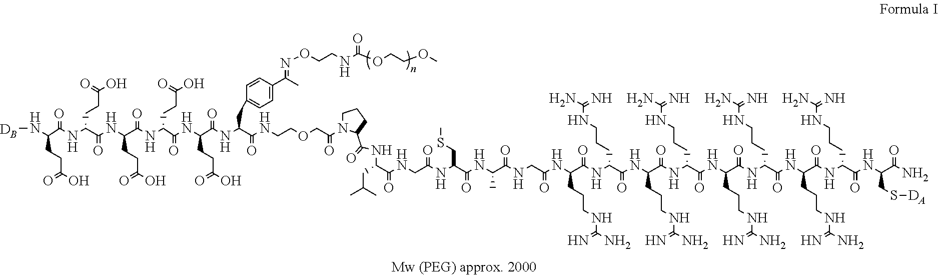

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the D_{A} donor moiety in the compound of Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, D_{A} and D_{B} are, respectively, selected from Cy5 and Cy7, Cy5 and IRDye750, Cy5 and IRDye800, and Cy5 and ICG. In some embodiments, the tissue is intact in the subject. In some embodiments, the tissue has been surgically excised from the subject.

Described herein, in certain embodiments, is a pharmaceutical composition configured in single dose form, wherein the single dose form comprises 1 mg to 16 mg of a compound of Formula I:

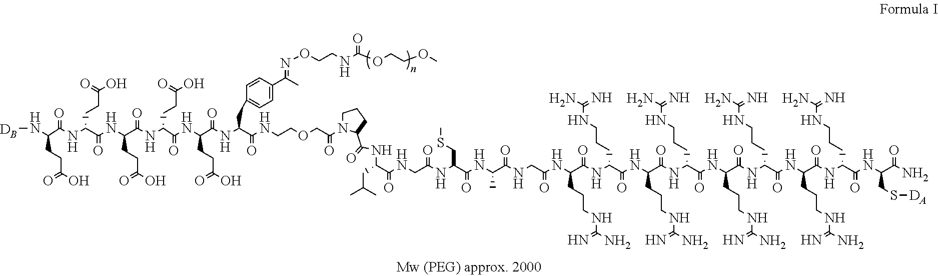

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, D_{A} and D_{B} are, respectively, selected from Cy5 and Cy7, Cy5 and IRDye750, Cy5 and IRDye800, and Cy5 and ICG.

### SDM-25

Described herein, in certain embodiments, are methods of surgically excising, evaluating, and imaging tissue, such as tissue comprising a plurality of cancerous cells, using the selective delivery molecule represented by SDM-25:

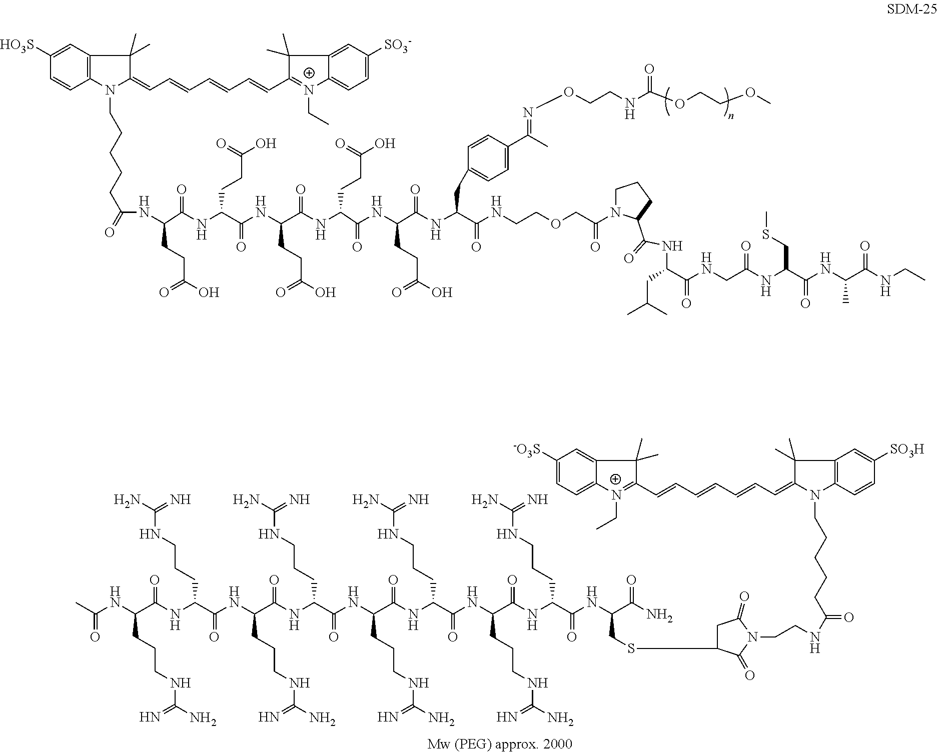

The selective delivery molecule SDM-25 allows for the targeted delivery of imaging agents to specific cells and/or tissues. SDM-25 is composed of a peptide of 22 amino acids, 2 fluorescent indocarbocyanine dyes, and an approximately 2 kDa polyethylene glycol (PEG) moiety conjugated to the peptide.

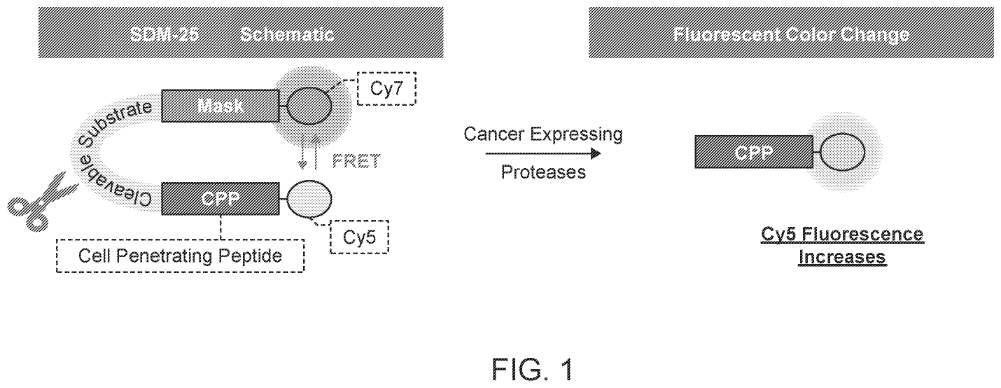

The 2 indocarbocyanine dyes of SDM-25 are far-red or near-infrared (NIR) fluorescent dyes (Cy5 and Cy7) that undergo Förster Resonance Energy Transfer (FRET). This energy transfer between the 2 dyes is disrupted upon hydrolysis of the peptide by proteases, resulting in a large fluorescence ratio change generated from both dyes (increase in Cy5 and decrease in Cy7) that can be visualized by fluorescence camera systems. The tumor tissue and surrounding microenvironment contains elevated protease activity due, in part, to increased secretion of matrix metalloproteinases (MMPs) from cancer and stromal cells; SDM-25 is a substrate for, and is hydrolyzed by, these proteases, resulting in a high fluorescence signal at tumors and metastases compared to background tissue that can be visualized by fluorescence camera systems (FIG. 1).

Disclosed herein, in certain embodiments, is a method of surgically excising a plurality of cancerous cells from a subject in need thereof, comprising: (i) administering to the subject an effective amount of SDM-25,

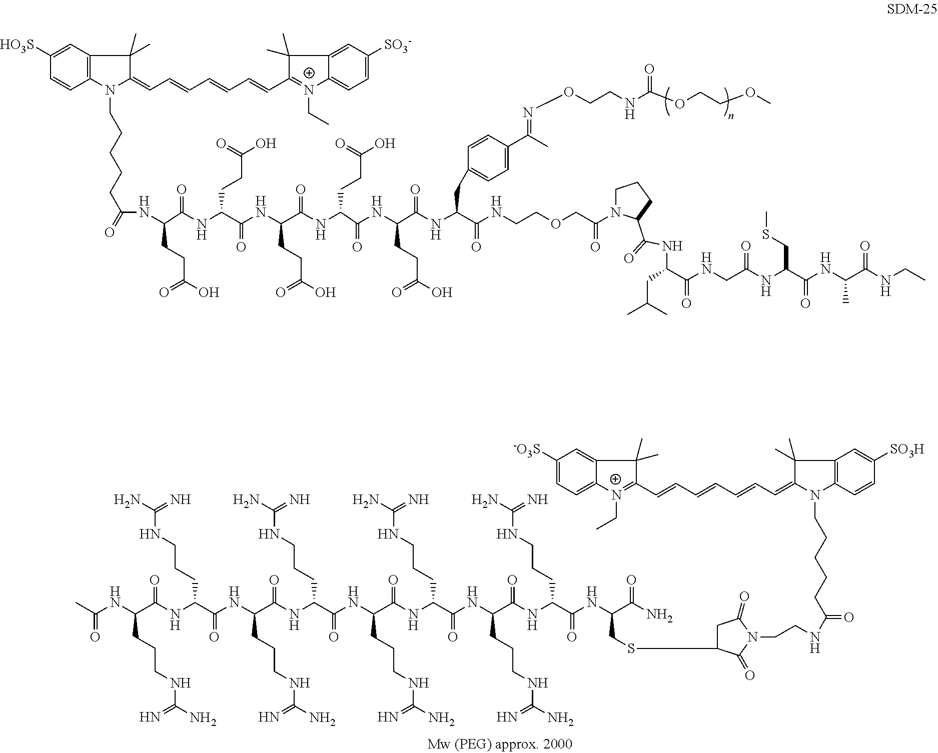

or a pharmaceutically acceptable salt thereof, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Also disclosed herein, in certain embodiments, is a method of surgically excising a plurality of cancerous cells from a subject in need thereof, comprising: (i) administering to the subject an effective amount of SDM-25,

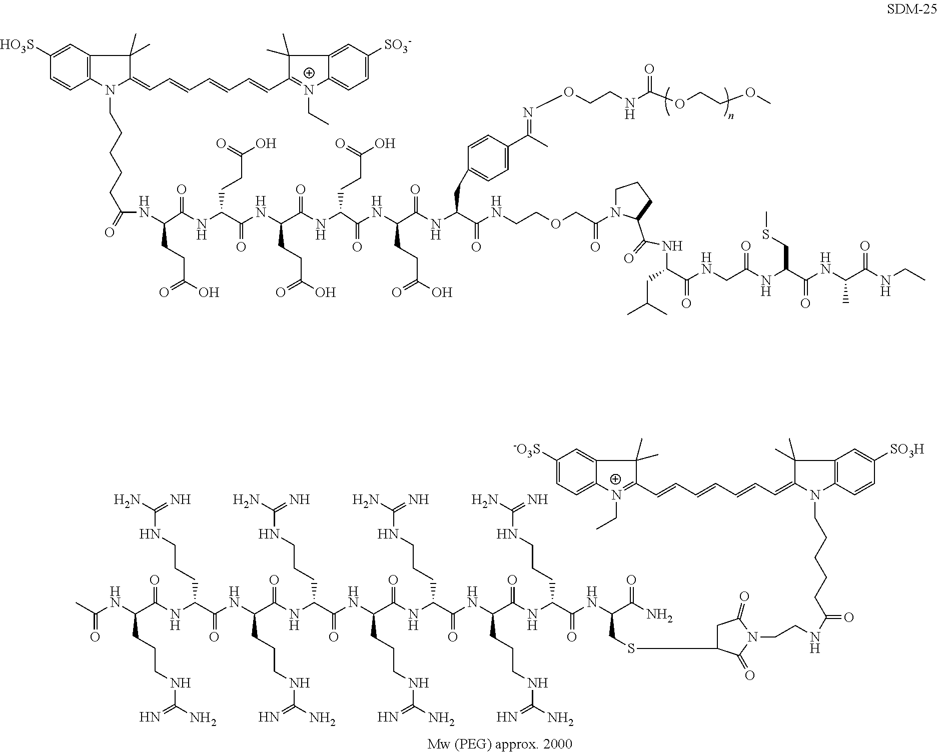

or a pharmaceutically acceptable salt thereof, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, prior to surgery, the additional tissue excised from the surgical cavity contacted the margin of the tissue comprising the plurality of cancerous cells. In some embodiments, the method comprises surgically excising the additional tissue across the full perimeter of the surgical cavity.

Additionally disclosed herein, in certain embodiments, is a method of evaluating a tissue suspected of having a plurality of cancerous cells, comprising: (i) administering to the subject an effective amount of SDM-25,

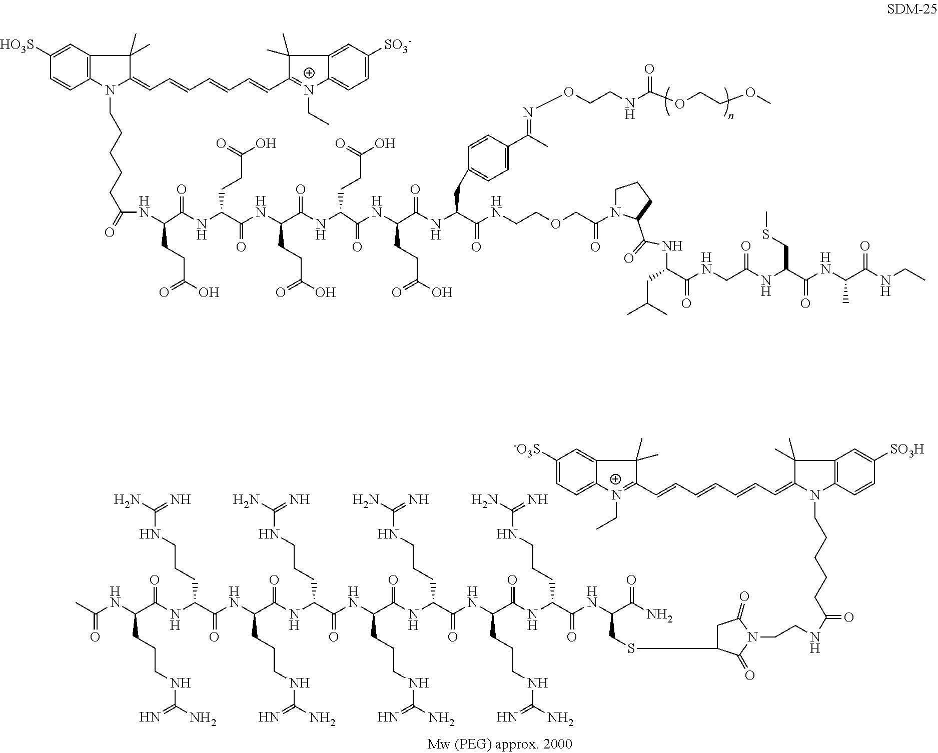

or a pharmaceutically acceptable salt thereof, at least 12 hours prior to surgery; (ii) contacting the tissue with light having at least one excitation wavelength of the Cy5 dye moiety in SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the tissue is intact in the subject. In some embodiments, the tissue has been surgically excised from the subject.

Additionally disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of SDM-25:

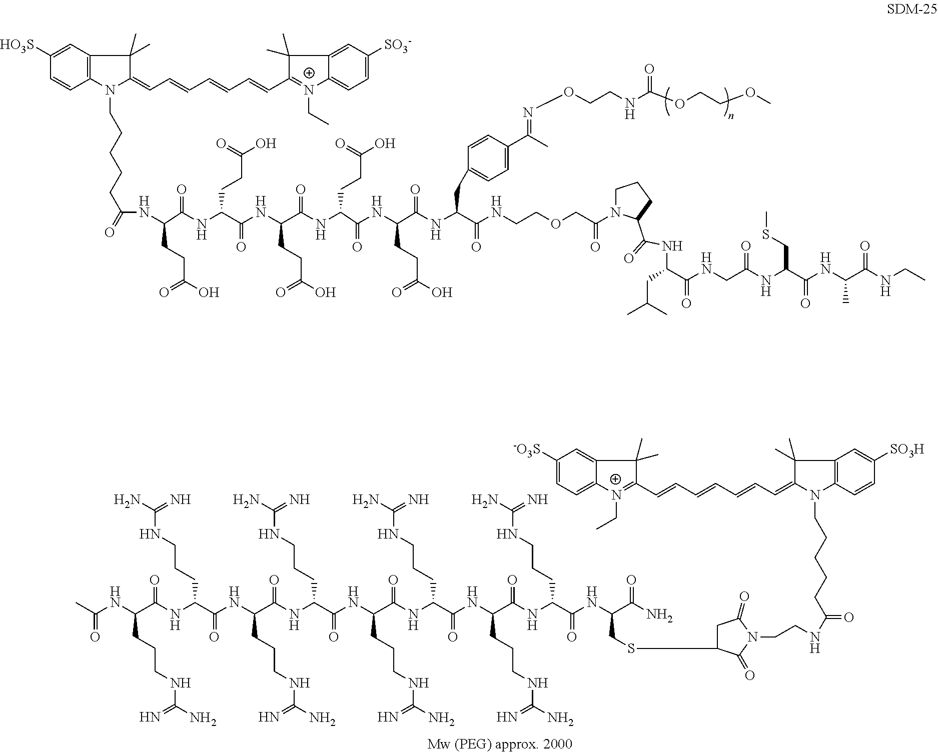

or a pharmaceutically acceptable salt thereof at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells.

Described herein, in certain embodiments, is a pharmaceutical composition configured in a single dose form, wherein the single dose form comprises 1 mg to 16 mg of a compound of SDM-25:

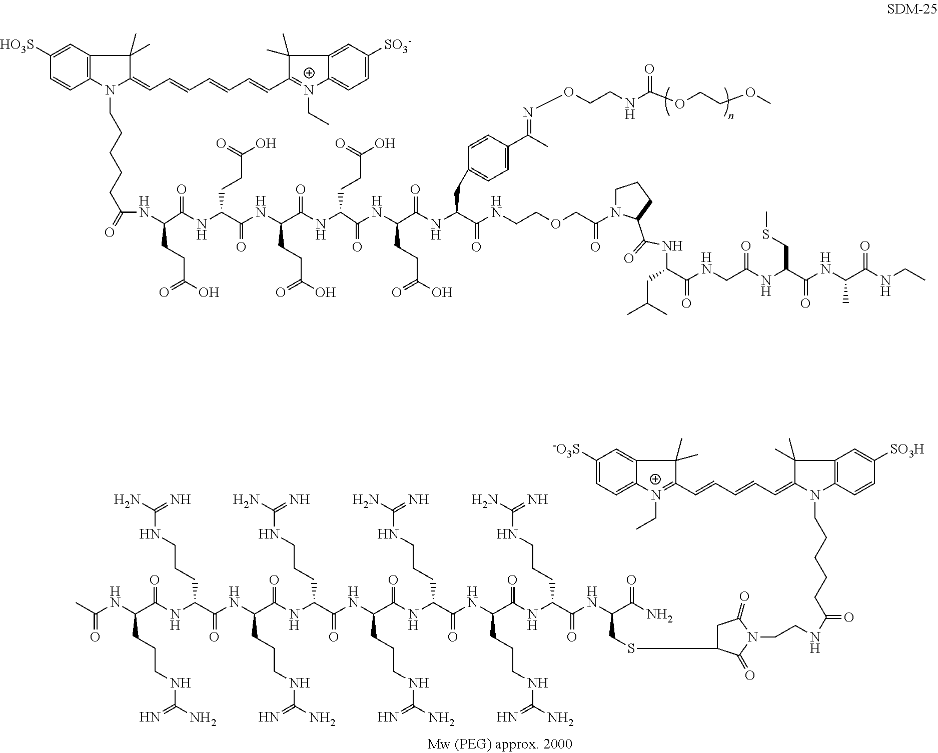

or a pharmaceutically acceptable salt thereof. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

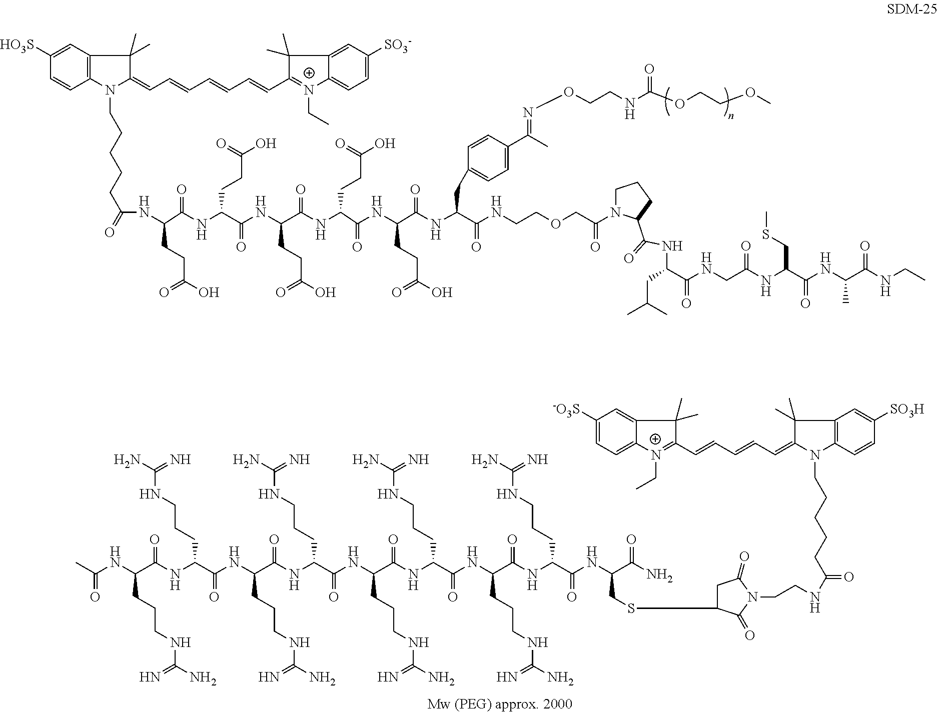

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

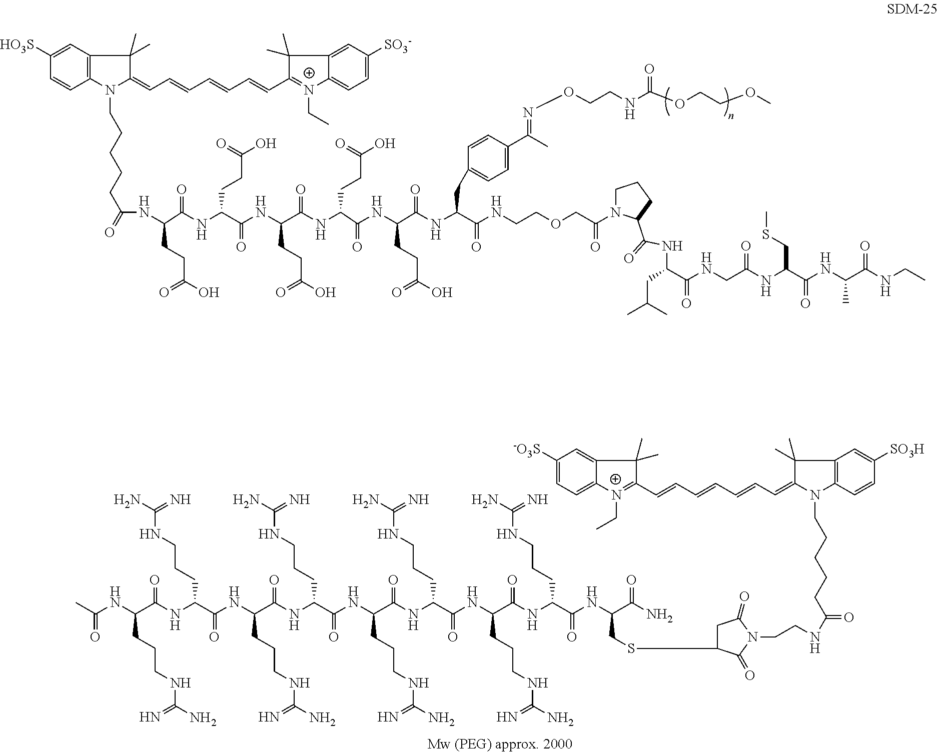

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, prior to surgery, the additional tissue excised from the surgical cavity contacted the margin of the tissue comprising the plurality of cancerous cells. In some embodiments, the use comprises surgically excising the additional tissue across the full perimeter of the surgical cavity.

Described herein, in certain embodiments, is a pharmaceutical composition configured in single dose form, wherein the single dose form comprises 1 mg to 16 mg of SDM-25:

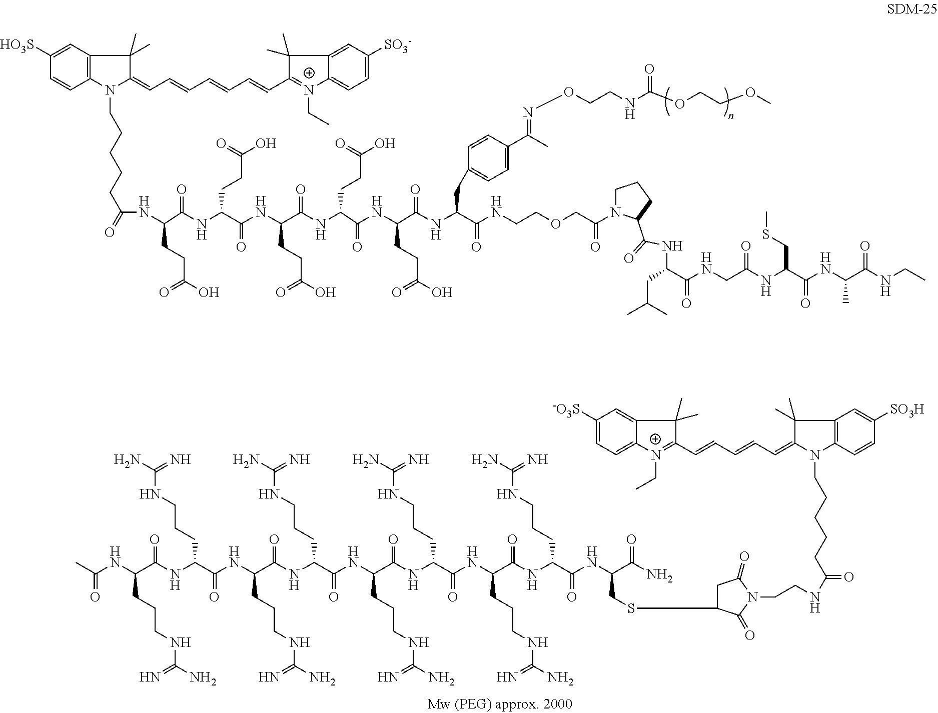

or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the Cy5 dye moiety of SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, the tissue is intact in the subject. In some embodiments, the tissue has been surgically excised from the subject.

Described herein, in certain embodiments, is a pharmaceutical composition configured in single dose form, wherein the single dose form comprises 1 mg to 16 mg of SDM-25:

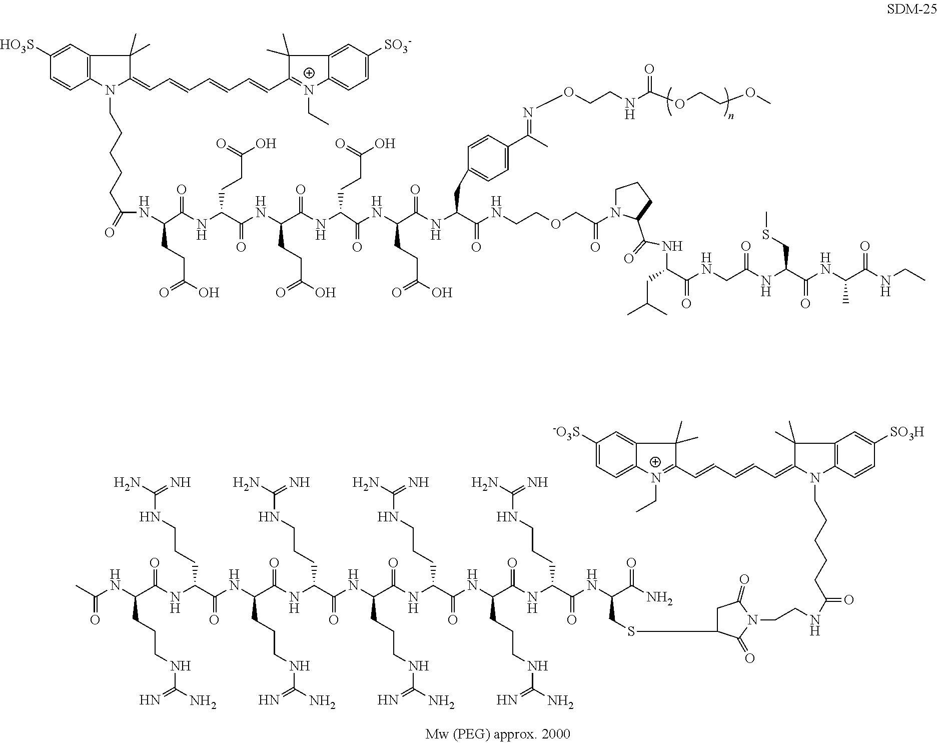

or a pharmaceutically acceptable salt thereof, for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

### Dosing and Dosing Schedule

SDM-25 comprises both a Cy5 fluorescence moiety and a Cy7 fluorescence moiety. When held in close proximity, e.g., as part of SDM-25, the Cy5 and Cy7 moieties undergo Förster Resonance Energy Transfer (FRET). When SDM-25 is disrupted by the hydrolysis of the linker by the proteases associated with the tumor microenvironment, the energy transfer between the 2 dyes is disrupted, resulting in a large fluorescence ratio change generated from both dyes (increase in Cy5 and decrease in Cy7) that can be visualized by fluorescence camera systems. The inventors have unexpectedly discovered that the intraoperative identification of positive and close margins, while maintaining low false positive results and significantly reducing false negative results, requires that the subject be administered SDM-25 at least 12 hours prior to surgery, (e.g., between 12 and 20 hours prior to surgery). This timing window allows sufficient amount of SDM-25 to undergo hydrolysis such that the Cy5 portion accumulates in the areas of lower cancerous cells or in cancer types with reduced MMP expression (thus reducing false negatives and optimization of finding cancer), while also allowing more time to systemically clear SDM-25 from the body and reducing background in surrounding tissues which also limits cancer detection. Some tumors, particularly those that express lower levels of MMPs (such as ductal carcinoma in situ (DCIS)) and in small, low volume tumors, require more time to allow the lower amount of MMP activity to cleave SDM-25 and to generate a sufficiently robust fluorescent signal that can be reliably detected and differentiated from that observed in nonmalignant tissue. It has additionally been found that a dose of 8 mg SDM-25 gives better separation between cancer-positive and cancer-negative tissue for both fluorescence ratio and intensity to distinguish tumors from adjacent tissue over higher doses that result in brighter signals.

Described herein, in certain embodiments, is a method of surgically excising a plurality of cancerous cells from a subject in need thereof, comprising: (i) administering to the subject an effective amount of a compound of Formula I,

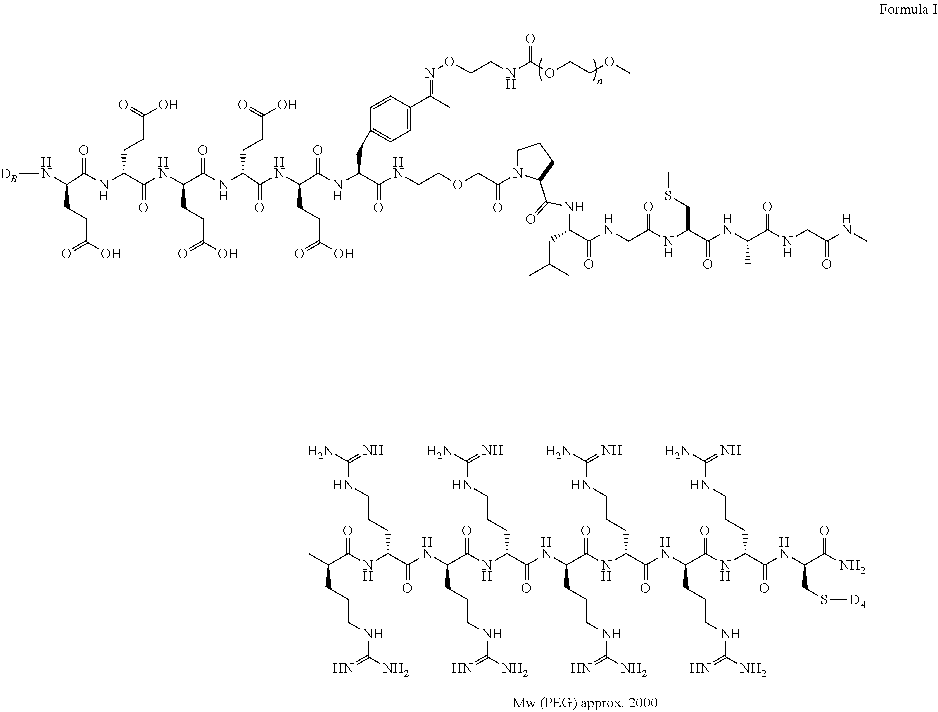

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the compound of Formula I or pharmaceutically acceptable salt thereof is administered to the subject at least about 14, 15, 16, 17, 18, 19, 20, or 21 hours prior to surgery. In some embodiments, the method comprises administering 1 mg to 16 mg of the compound of Formula I or pharmaceutically acceptable salt thereof to the subject. In some embodiments, the method comprises administering 8 mg of the compound of Formula I or pharmaceutically acceptable salt thereof to the subject.

Also disclosed herein, in certain embodiments, is a method of surgically excising a plurality of cancerous cells from a subject in need thereof, comprising: (i) administering to the subject an effective amount of a compound of Formula I,

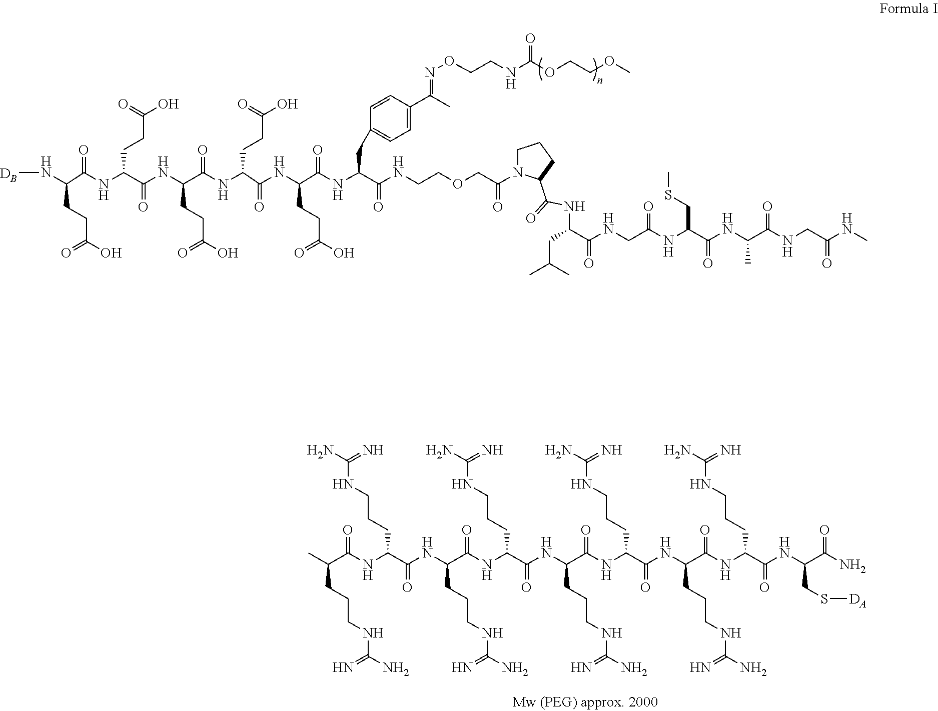

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the compound of Formula I or pharmaceutically acceptable salt thereof is administered to the subject at least about 14, 15, 16, 17, 18, 19, 20, or 21 hours prior to surgery. In some embodiments, the method comprises administering 1 mg to 16 mg of the compound of Formula I or pharmaceutically acceptable salt thereof to the subject. In some embodiments, the method comprises administering 8 mg of the compound of Formula I or pharmaceutically acceptable salt thereof to the subject.

Additionally disclosed herein, in certain embodiments, is a method of evaluating a tissue suspected of having a plurality of cancerous cells, comprising: (i) administering to the subject an effective amount of a compound of Formula I,

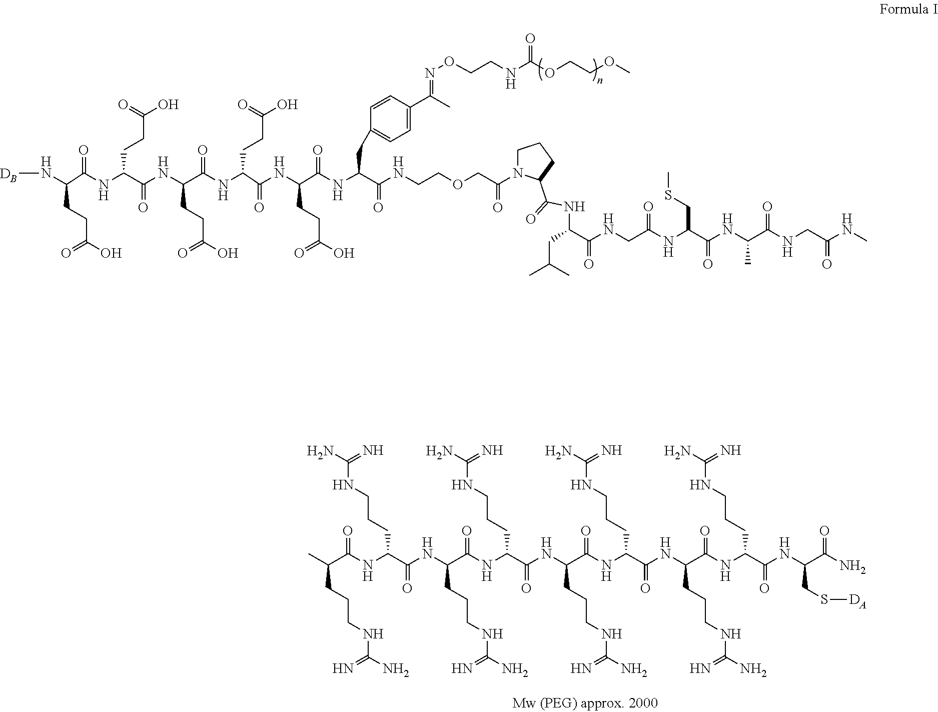

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) contacting the tissue with light having at least one excitation wavelength of the D_{A} donor moiety in Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the compound of Formula I or pharmaceutically acceptable salt thereof is administered to the subject at least about 14, 15, 16, 17, 18, 19, 20, or 21 hours prior to surgery. In some embodiments, the method comprises administering 1 mg to 16 mg of the compound of Formula I or pharmaceutically acceptable salt thereof to the subject. In some embodiments, the method comprises administering 8 mg of the compound of Formula I or pharmaceutically acceptable salt thereof to the subject.

Additionally disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of a compound of Formula I:

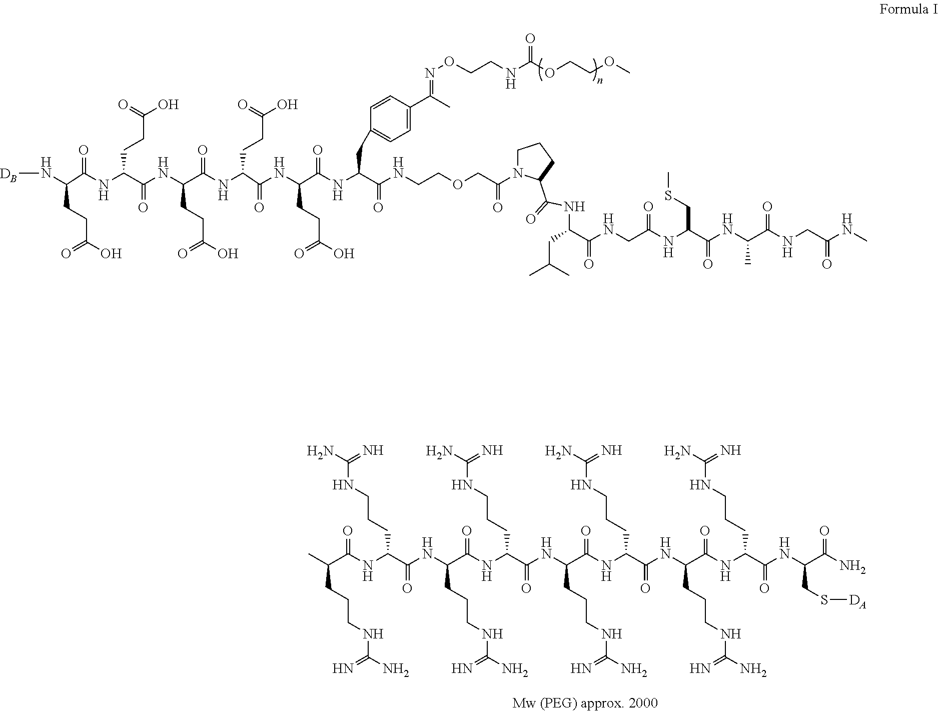

wherein: D_{A} and D_{B} are, respectively, a donor moiety and acceptor moiety that are capable of participating in Förster Resonance Energy Transfer; or a pharmaceutically acceptable salt thereof at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the compound of Formula I or pharmaceutically acceptable salt thereof is administered to the subject at least about 14, 15, 16, 17, 18, 19, 20, or 21 hours prior to surgery. In some embodiments, the method comprises administering 1 mg to 16 mg of the compound of Formula I or pharmaceutically acceptable salt thereof to the subject. In some embodiments, the method comprises administering 8 mg of the compound of Formula I or pharmaceutically acceptable salt thereof to the subject.

In some embodiments, the compound of Formula I or pharmaceutically acceptable salt thereof is administered to the subject at least about 14 hours prior to surgery. In some embodiments, the compound of Formula I or pharmaceutically acceptable salt thereof is administered to the subject at least about 15 hours prior to surgery. In some embodiments, the compound of Formula I or pharmaceutically acceptable salt thereof is administered to the subject at least about 16 hours prior to surgery. In some embodiments, the compound of Formula I or pharmaceutically acceptable salt thereof is administered to the subject at least about 17 hours prior to surgery. In some embodiments, the compound of Formula I or pharmaceutically acceptable salt thereof is administered to the subject at least about 18 hours prior to surgery.

In some embodiments, the compound of Formula I or pharmaceutically acceptable salt thereof is administered to the subject between about 14 and about 20 hours prior to surgery. In some embodiments, the compound of Formula I or pharmaceutically acceptable salt thereof is administered to the subject between about 14 and about 19 hours prior to surgery. In some embodiments, the compound of Formula I or pharmaceutically acceptable salt thereof is administered to the subject between about 14 and about 18 hours prior to surgery. In some embodiments, the compound of Formula I or pharmaceutically acceptable salt thereof is administered to the subject between about 14 and about 17 hours prior to surgery. In some embodiments, the compound of Formula I or pharmaceutically acceptable salt thereof is administered to the subject between about 14 and about 16 hours prior to surgery. In some embodiments, the compound of Formula I or pharmaceutically acceptable salt thereof is administered to the subject between about 14 and about 15 hours prior to surgery.

In some embodiments, the compound of Formula I or pharmaceutically acceptable salt thereof is administered to the subject between about 15 and about 20 hours prior to surgery. In some embodiments, the compound of Formula I or pharmaceutically acceptable salt thereof is administered to the subject between about 16 and about 20 hours prior to surgery. In some embodiments, the compound of Formula I or pharmaceutically acceptable salt thereof is administered to the subject between about 17 and about 20 hours prior to surgery. In some embodiments, the compound of Formula I or pharmaceutically acceptable salt thereof is administered to the subject between about 18 and about 20 hours prior to surgery. In some embodiments, the compound of Formula I or pharmaceutically acceptable salt thereof is administered to the subject between about 19 and about 20 hours prior to surgery.

In some embodiments, the compound of Formula I or pharmaceutically acceptable salt thereof is administered to the subject between about 15 and about 19 hours prior to surgery. In some embodiments, the compound of Formula I or pharmaceutically acceptable salt thereof is administered to the subject between about 16 and about 18 hours prior to surgery.

In some embodiments, the compound of Formula I or pharmaceutically acceptable salt thereof is not administered to the subject more than 20 hours before surgery. In some embodiments, the compound of Formula I or pharmaceutically acceptable salt thereof is not administered to the subject more than 20.5 hours before surgery. In some embodiments, the compound of Formula I or pharmaceutically acceptable salt thereof is not administered to the subject more than 21 hours before surgery.

Disclosed herein, in certain embodiments, is a method of surgically excising a plurality of cancerous cells from a subject in need thereof, comprising: (i) administering to the subject an effective amount of SDM-25,

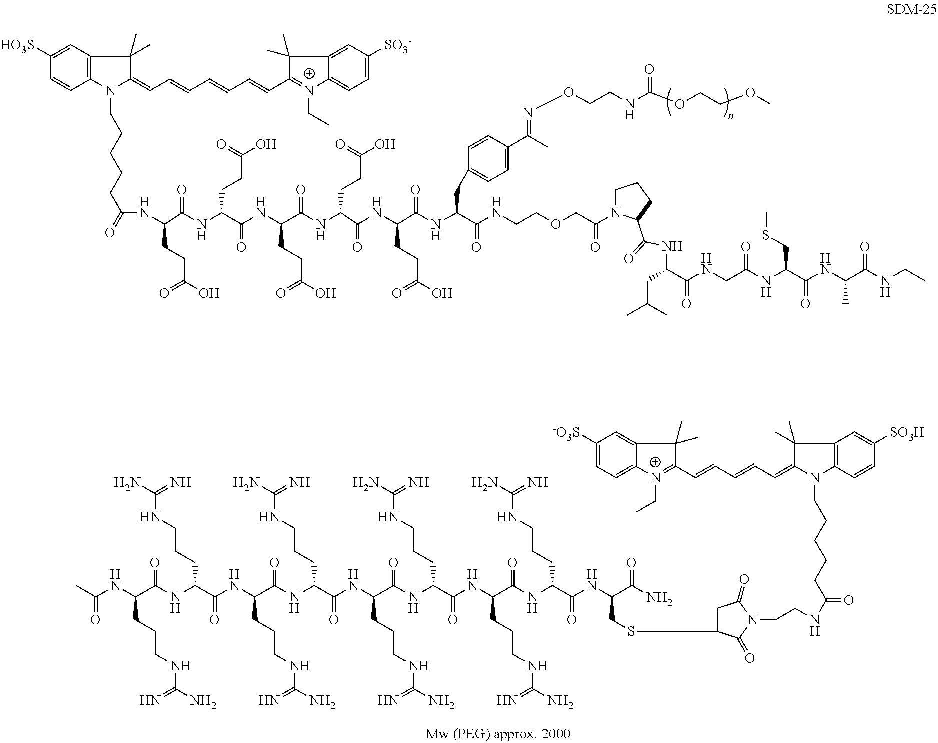

or a pharmaceutically acceptable salt thereof, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, SDM-25 or pharmaceutically acceptable salt thereof is administered to the subject at least about 14, 15, 16, 17, 18, 19, 20, or 21 hours prior to surgery. In some embodiments, the method comprises administering 1 mg to 16 mg of SDM-25 or pharmaceutically acceptable salt thereof to the subject. In some embodiments, the method comprises administering 8 mg of SDM-25 or pharmaceutically acceptable salt thereof to the subject.

Also disclosed herein, in certain embodiments, is a method of surgically excising a plurality of cancerous cells from a subject in need thereof, comprising: (i) administering to the subject an effective amount of SDM-25,

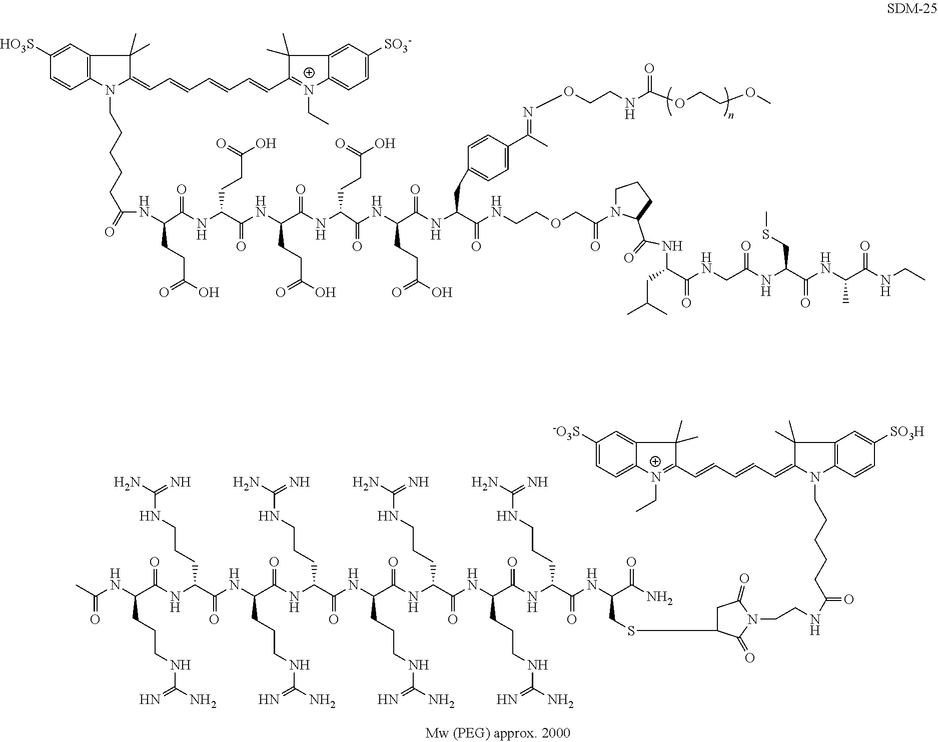

or a pharmaceutically acceptable salt thereof, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, SDM-25 or pharmaceutically acceptable salt thereof is administered to the subject at least about 14, 15, 16, 17, 18, 19, 20, or 21 hours prior to surgery. In some embodiments, the method comprises administering 1 mg to 16 mg of SDM-25 or pharmaceutically acceptable salt thereof to the subject. In some embodiments, the method comprises administering 8 mg of SDM-25 or pharmaceutically acceptable salt thereof to the subject.

Additionally disclosed herein, in certain embodiments, is a method of evaluating a tissue suspected of having a plurality of cancerous cells, comprising: (i) administering to the subject an effective amount of SDM-25,

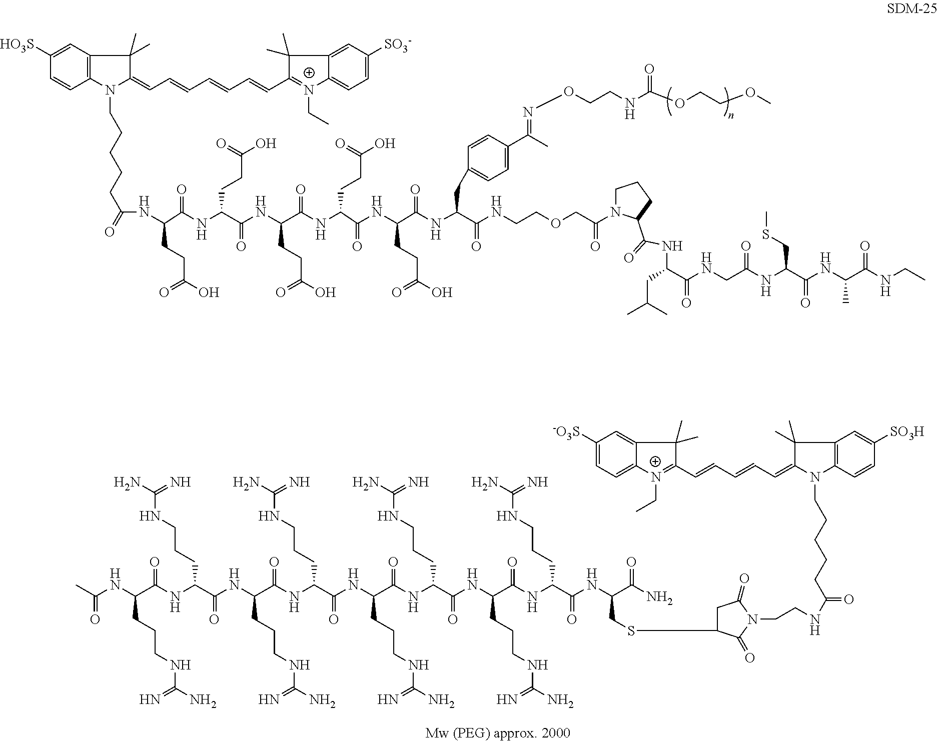

or a pharmaceutically acceptable salt thereof, at least 12 hours prior to surgery; (ii) contacting the tissue with light having at least one excitation wavelength of the Cy5 dye moiety in SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, SDM-25 or pharmaceutically acceptable salt thereof is administered to the subject at least about 14, 15, 16, 17, 18, 19, 20, or 21 hours prior to surgery. In some embodiments, the method comprises administering 1 mg to 16 mg of SDM-25 or pharmaceutically acceptable salt thereof to the subject. In some embodiments, the method comprises administering 8 mg of SDM-25 or pharmaceutically acceptable salt thereof to the subject.

Additionally disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of SDM-25:

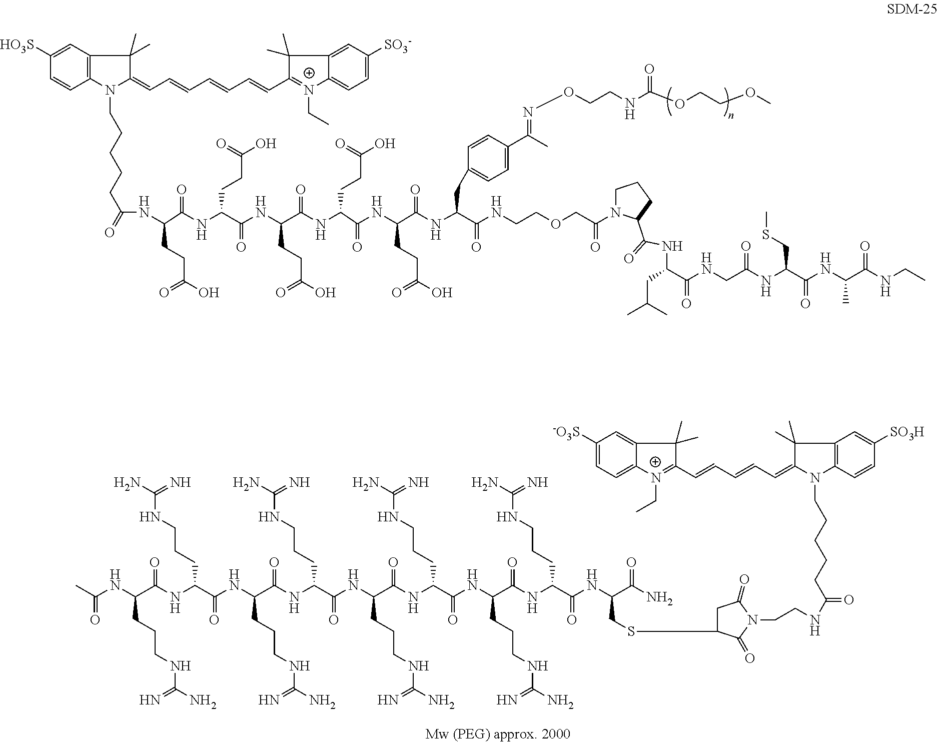

or a pharmaceutically acceptable salt thereof at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells. In some embodiments, SDM-25 or pharmaceutically acceptable salt thereof is administered to the subject at least about 14, 15, 16, 17, 18, 19, 20, or 21 hours prior to surgery. In some embodiments, the method comprises administering 1 mg to 16 mg of SDM-25 or pharmaceutically acceptable salt thereof to the subject. In some embodiments, the method comprises administering 8 mg of SDM-25 or pharmaceutically acceptable salt thereof to the subject.

In some embodiments, SDM-25 or pharmaceutically acceptable salt thereof is administered to the subject at least about 14 hours prior to surgery. In some embodiments, SDM-25 or pharmaceutically acceptable salt thereof is administered to the subject at least about 15 hours prior to surgery. In some embodiments, SDM-25 or pharmaceutically acceptable salt thereof is administered to the subject at least about 16 hours prior to surgery. In some embodiments, SDM-25 or pharmaceutically acceptable salt thereof is administered to the subject at least about 17 hours prior to surgery. In some embodiments, SDM-25 or pharmaceutically acceptable salt thereof is administered to the subject at least about 18 hours prior to surgery.

In some embodiments, SDM-25 or pharmaceutically acceptable salt thereof is administered to the subject between about 14 and about 20 hours prior to surgery. In some embodiments, SDM-25 or pharmaceutically acceptable salt thereof is administered to the subject between about 14 and about 19 hours prior to surgery. In some embodiments, SDM-25 or pharmaceutically acceptable salt thereof is administered to the subject between about 14 and about 18 hours prior to surgery. In some embodiments, SDM-25 or pharmaceutically acceptable salt thereof is administered to the subject between about 14 and about 17 hours prior to surgery. In some embodiments, SDM-25 or pharmaceutically acceptable salt thereof is administered to the subject between about 14 and about 16 hours prior to surgery. In some embodiments, SDM-25 or pharmaceutically acceptable salt thereof is administered to the subject between about 14 and about 15 hours prior to surgery.

In some embodiments, SDM-25 or pharmaceutically acceptable salt thereof is administered to the subject between about 15 and about 20 hours prior to surgery. In some embodiments, SDM-25 or pharmaceutically acceptable salt thereof is administered to the subject between about 16 and about 20 hours prior to surgery. In some embodiments, SDM-25 or pharmaceutically acceptable salt thereof is administered to the subject between about 17 and about 20 hours prior to surgery. In some embodiments, SDM-25 or pharmaceutically acceptable salt thereof is administered to the subject between about 18 and about 20 hours prior to surgery. In some embodiments, SDM-25 or pharmaceutically acceptable salt thereof is administered to the subject between about 19 and about 20 hours prior to surgery.

In some embodiments, SDM-25 or pharmaceutically acceptable salt thereof is administered to the subject between about 15 and about 19 hours prior to surgery. In some embodiments, SDM-25 or pharmaceutically acceptable salt thereof is administered to the subject between about 16 and about 18 hours prior to surgery.

In some embodiments, SDM-25 or pharmaceutically acceptable salt thereof is not administered to the subject more than 20 hours before surgery. In some embodiments, SDM-25 or pharmaceutically acceptable salt thereof is not administered to the subject more than 20.5 hours before surgery. In some embodiments, SDM-25 or pharmaceutically acceptable salt thereof is not administered to the subject more than 21 hours before surgery.

Pharmaceutical compositions configured as dosage forms for use in the methods described herein are also provided. Described herein, in certain embodiments, is a pharmaceutical composition configured in single dose form, wherein the single dose form comprises 1 mg to 16 mg of a compound of Formula I:

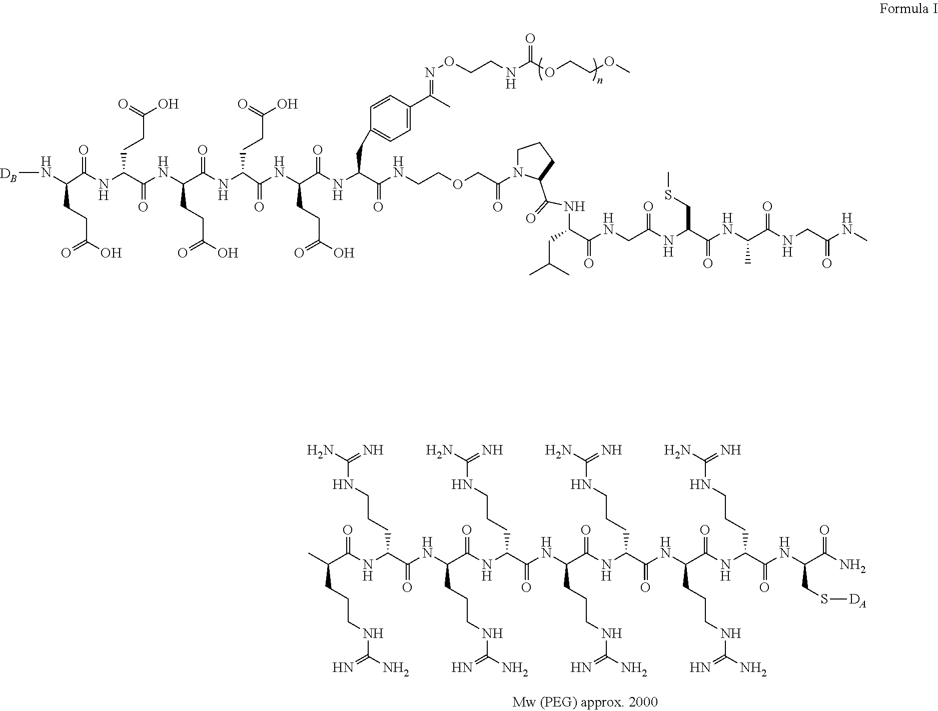

wherein: D_{A} and D_{B} are, respectively, a donor moiety and acceptor moiety that are capable of participating in Förster Resonance Energy Transfer; or a pharmaceutically acceptable salt thereof. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

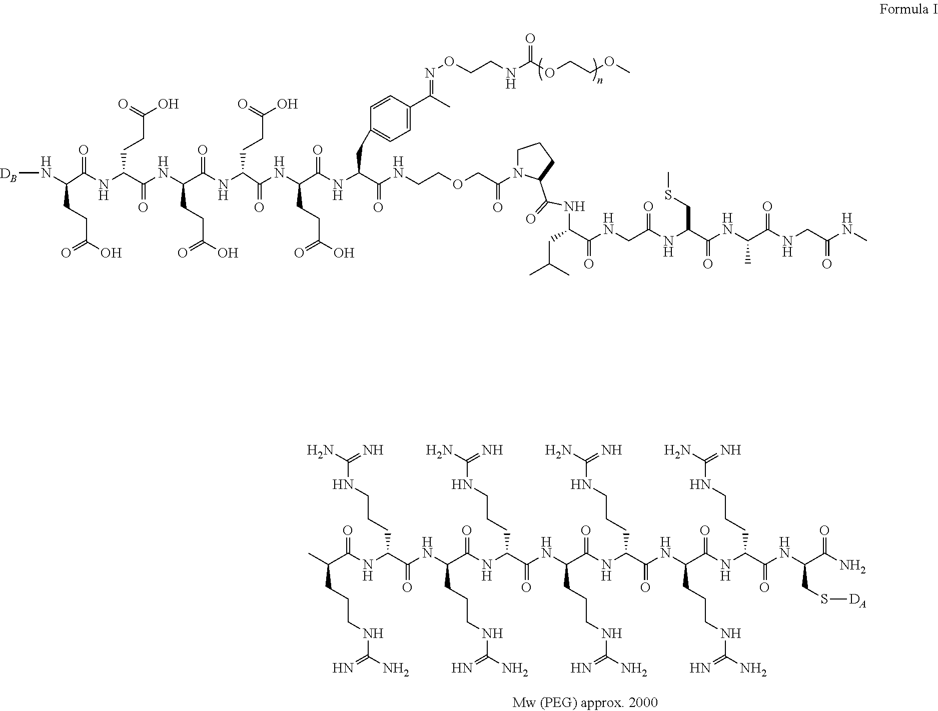

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the compound of Formula I or pharmaceutically acceptable salt thereof is administered to the subject at least about 14, 15, 16, 17, 18, 19, 20, or 21 hours prior to surgery.

Described herein, in certain embodiments, is a pharmaceutical composition configured in single dose form, wherein the single dose form comprises 1 mg to 16 mg of a compound of Formula I:

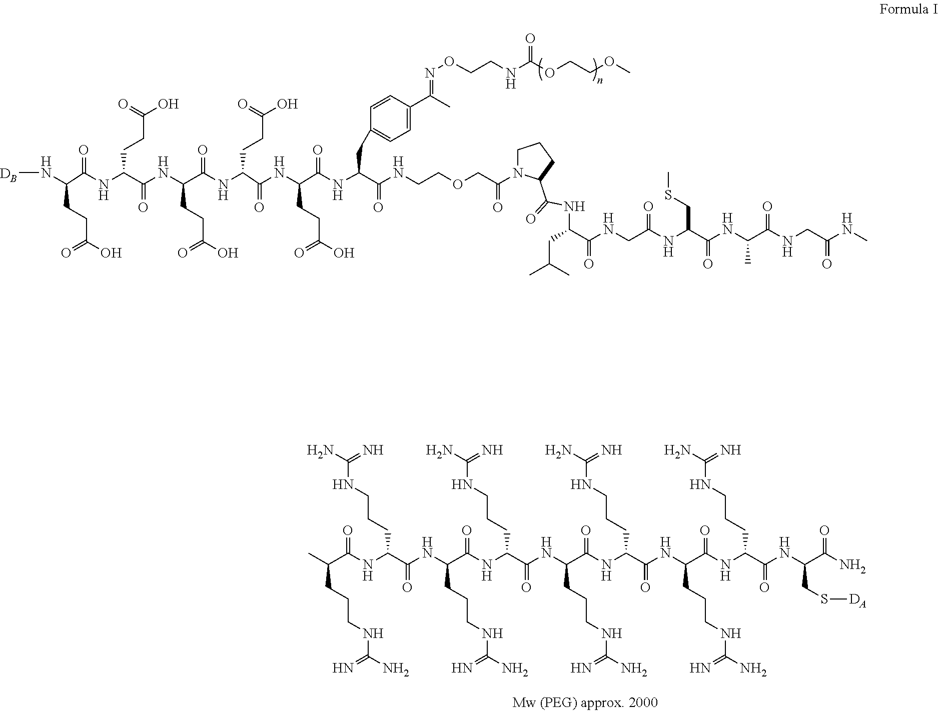

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition; at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the compound of Formula I or pharmaceutically acceptable salt thereof is administered to the subject at least about 14, 15, 16, 17, 18, 19, 20, or 21 hours prior to surgery.

Described herein, in certain embodiments, is a pharmaceutical composition configured in single dose form, wherein the single dose form comprises 1 mg to 16 mg of a compound of Formula I:

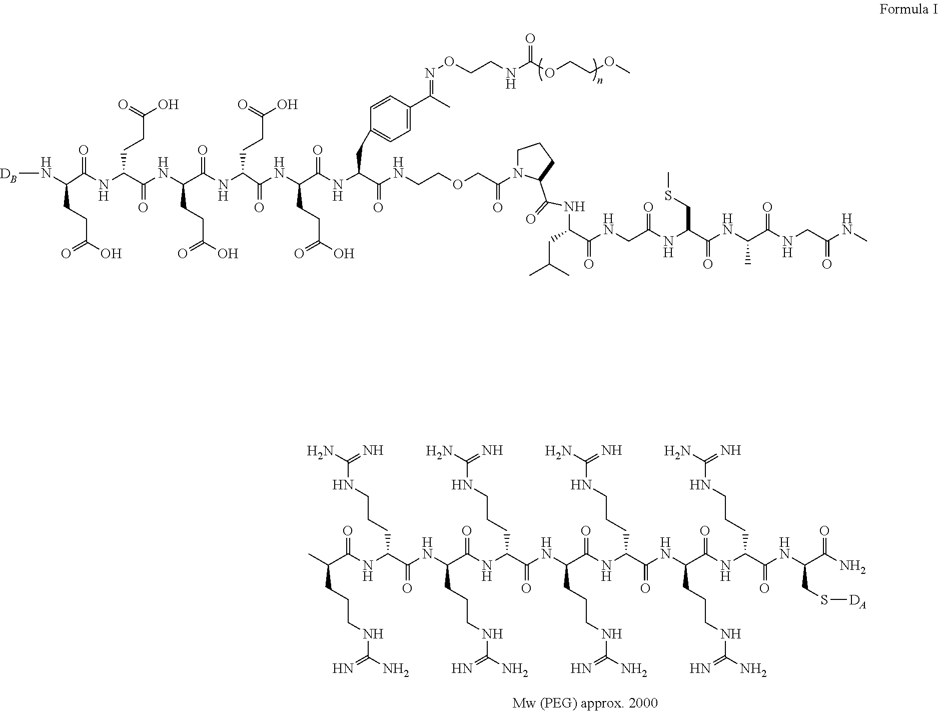

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the D_{A} donor moiety in the compound of Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the compound of Formula I or pharmaceutically acceptable salt thereof is administered to the subject at least about 14, 15, 16, 17, 18, 19, 20, or 21 hours prior to surgery.

Described herein, in certain embodiments, is a pharmaceutical composition configured in single dose form, wherein the single dose form comprises 1 mg to 16 mg of a compound of Formula I:

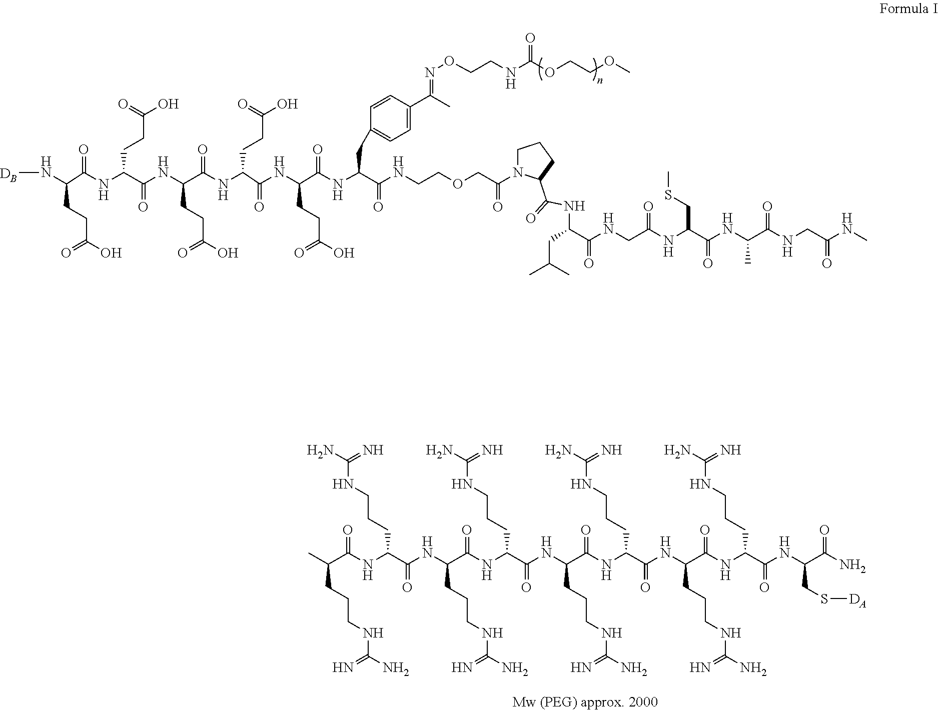

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the compound of Formula I or pharmaceutically acceptable salt thereof is administered to the subject at least about 14, 15, 16, 17, 18, 19, 20, or 21 hours prior to surgery.

In some embodiments, the compound of Formula I or pharmaceutically acceptable salt thereof is administered to the subject at least about 14 hours prior to surgery. In some embodiments, the compound of Formula I or pharmaceutically acceptable salt thereof is administered to the subject at least about 15 hours prior to surgery. In some embodiments, the compound of Formula I or pharmaceutically acceptable salt thereof is administered to the subject at least about 16 hours prior to surgery. In some embodiments, the compound of Formula I or pharmaceutically acceptable salt thereof is administered to the subject at least about 17 hours prior to surgery. In some embodiments, the compound of Formula I or pharmaceutically acceptable salt thereof is administered to the subject at least about 18 hours prior to surgery.

In some embodiments, the compound of Formula I or pharmaceutically acceptable salt thereof is administered to the subject between about 14 and about 20 hours prior to surgery. In some embodiments, the compound of Formula I or pharmaceutically acceptable salt thereof is administered to the subject between about 14 and about 19 hours prior to surgery. In some embodiments, the compound of Formula I or pharmaceutically acceptable salt thereof is administered to the subject between about 14 and about 18 hours prior to surgery. In some embodiments, the compound of Formula I or pharmaceutically acceptable salt thereof is administered to the subject between about 14 and about 17 hours prior to surgery. In some embodiments, the compound of Formula I or pharmaceutically acceptable salt thereof is administered to the subject between about 14 and about 16 hours prior to surgery. In some embodiments, the compound of Formula I or pharmaceutically acceptable salt thereof is administered to the subject between about 14 and about 15 hours prior to surgery.

In some embodiments, the compound of Formula I or pharmaceutically acceptable salt thereof is administered to the subject between about 15 and about 20 hours prior to surgery. In some embodiments, the compound of Formula I or pharmaceutically acceptable salt thereof is administered to the subject between about 16 and about 20 hours prior to surgery. In some embodiments, the compound of Formula I or pharmaceutically acceptable salt thereof is administered to the subject between about 17 and about 20 hours prior to surgery. In some embodiments, the compound of Formula I or pharmaceutically acceptable salt thereof is administered to the subject between about 18 and about 20 hours prior to surgery. In some embodiments, the compound of Formula I or pharmaceutically acceptable salt thereof is administered to the subject between about 19 and about 20 hours prior to surgery.

In some embodiments, the compound of Formula I or pharmaceutically acceptable salt thereof is administered to the subject between about 15 and about 19 hours prior to surgery. In some embodiments, the compound of Formula I or pharmaceutically acceptable salt thereof is administered to the subject between about 16 and about 18 hours prior to surgery.

In some embodiments, the compound of Formula I or pharmaceutically acceptable salt thereof is not administered to the subject more than 20 hours before surgery. In some embodiments, the compound of Formula I or pharmaceutically acceptable salt thereof is not administered to the subject more than 20.5 hours before surgery. In some embodiments, the compound of Formula I or pharmaceutically acceptable salt thereof is not administered to the subject more than 21 hours before surgery.

Described herein, in certain embodiments, is a pharmaceutical composition configured in a single dose form, wherein the single dose form comprises 1 mg to 16 mg of a compound of SDM-25:

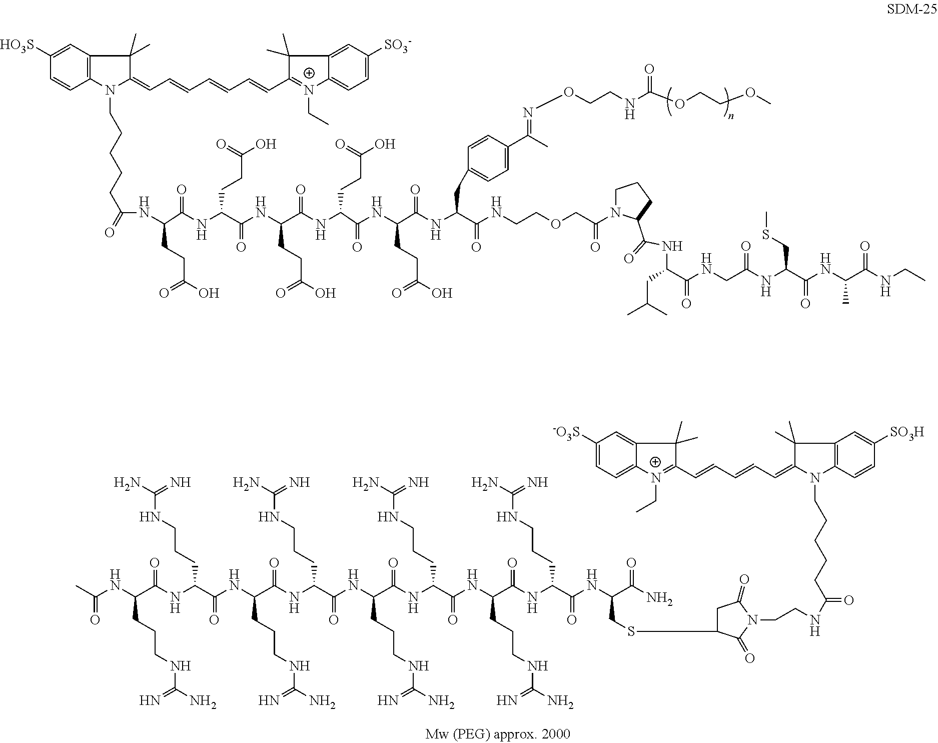

or a pharmaceutically acceptable salt thereof. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

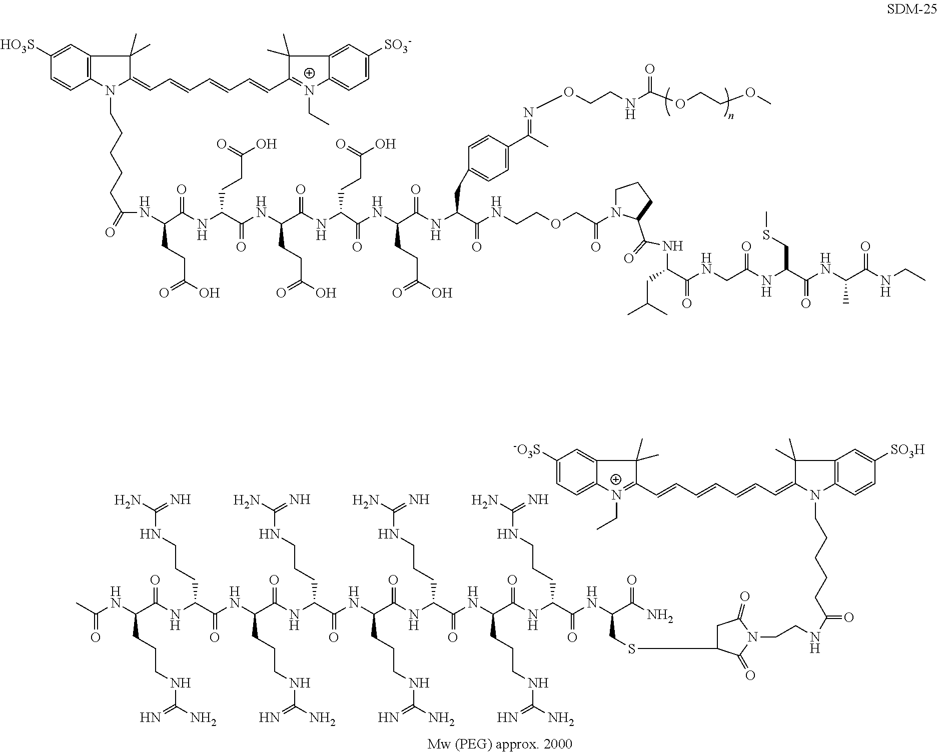

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, SDM-25 or pharmaceutically acceptable salt thereof is administered to the subject at least about 14, 15, 16, 17, 18, 19, 20, or 21 hours prior to surgery.

Described herein, in certain embodiments, is a pharmaceutical composition configured in single dose form, wherein the single dose form comprises 1 mg to 16 mg of SDM-25:

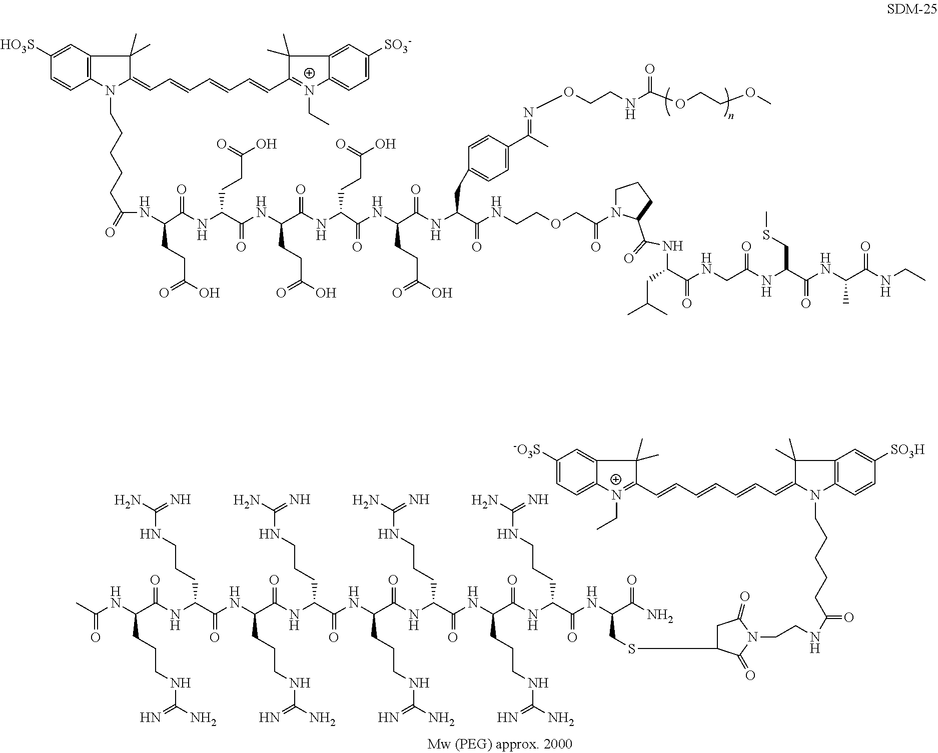

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, SDM-25 or pharmaceutically acceptable salt thereof is administered to the subject at least about 14, 15, 16, 17, 18, 19, 20, or 21 hours prior to surgery.

Described herein, in certain embodiments, is a pharmaceutical composition configured in single dose form, wherein the single dose form comprises 1 mg to 16 mg of SDM-25:

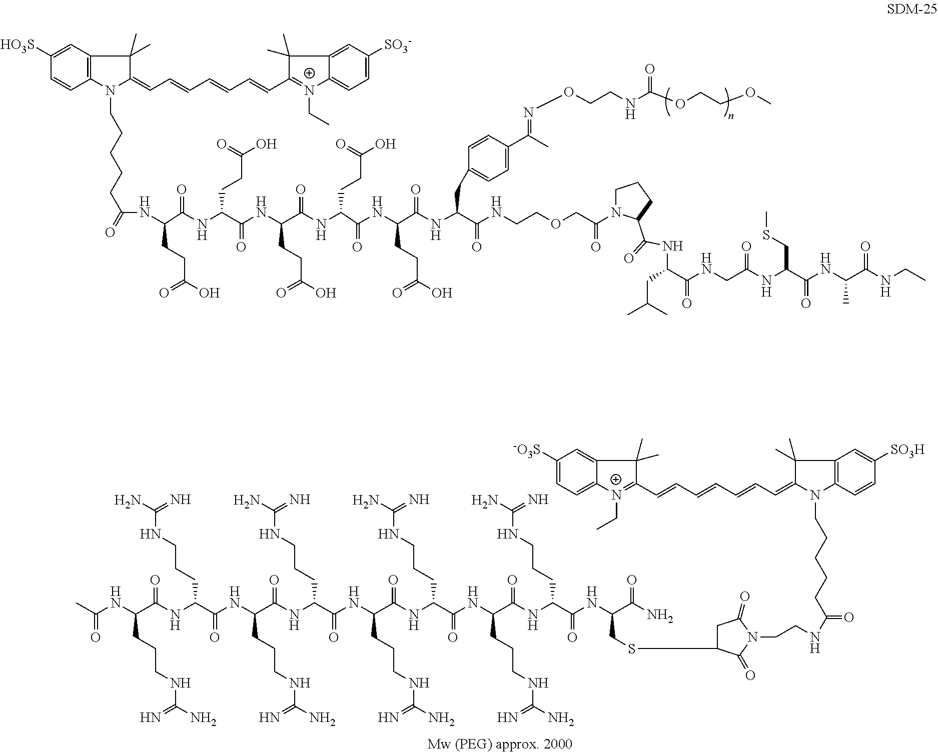

or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the Cy5 dye moiety of SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, SDM-25 or pharmaceutically acceptable salt thereof is administered to the subject at least about 14, 15, 16, 17, 18, 19, 20, or 21 hours prior to surgery.

Described herein, in certain embodiments, is a pharmaceutical composition configured in single dose form, wherein the single dose form comprises 1 mg to 16 mg of SDM-25:

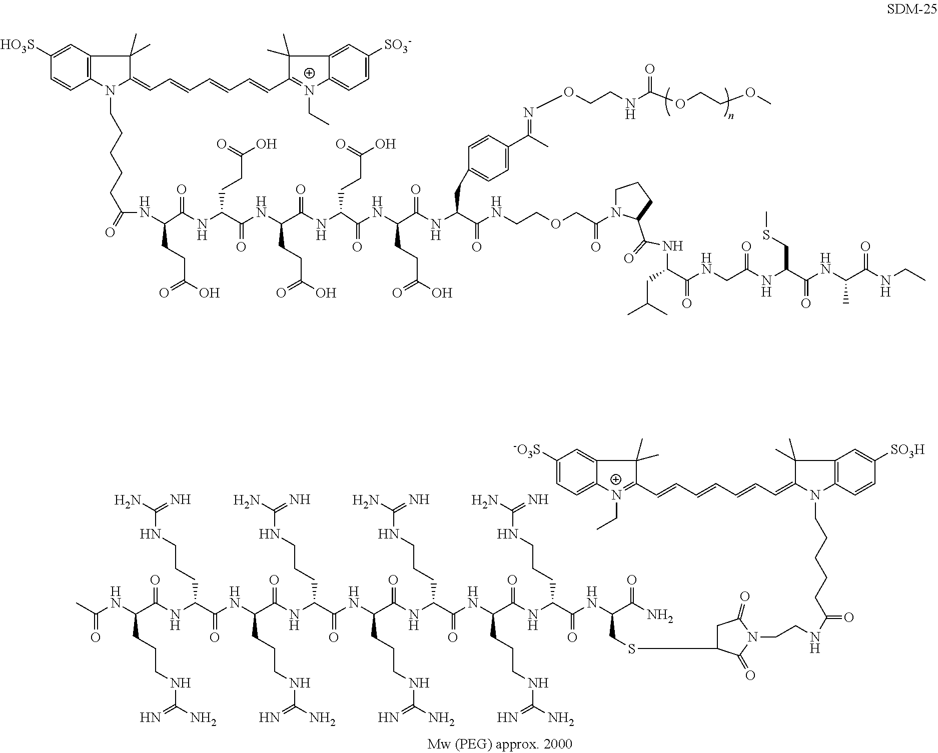

or a pharmaceutically acceptable salt thereof, for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, SDM-25 or pharmaceutically acceptable salt thereof is administered to the subject at least about 14, 15, 16, 17, 18, 19, 20, or 21 hours prior to surgery.

In some embodiments, SDM-25 or pharmaceutically acceptable salt thereof is administered to the subject between about 15 and about 20 hours prior to surgery. In some embodiments, SDM-25 or pharmaceutically acceptable salt thereof is administered to the subject between about 16 and about 20 hours prior to surgery. In some embodiments, SDM-25 or pharmaceutically acceptable salt thereof is administered to the subject between about 17 and about 20 hours prior to surgery. In some embodiments, SDM-25 or pharmaceutically acceptable salt thereof is administered to the subject between about 18 and about 20 hours prior to surgery. In some embodiments, SDM-25 or pharmaceutically acceptable salt thereof is administered to the subject between about 19 and about 20 hours prior to surgery.

In some embodiments, SDM-25 or pharmaceutically acceptable salt thereof is administered to the subject between about 15 and about 19 hours prior to surgery. In some embodiments, SDM-25 or pharmaceutically acceptable salt thereof is administered to the subject between about 16 and about 18 hours prior to surgery.

In some embodiments, SDM-25 or pharmaceutically acceptable salt thereof is not administered to the subject more than 20 hours before surgery. In some embodiments, SDM-25 or pharmaceutically acceptable salt thereof is not administered to the subject more than 20.5 hours before surgery. In some embodiments, SDM-25 or pharmaceutically acceptable salt thereof is not administered to the subject more than 21 hours before surgery.

### Administration

Described herein, in certain embodiments, is a method of surgically excising a plurality of cancerous cells from a subject in need thereof, comprising: (i) administering to the subject an effective amount of a compound of Formula I,

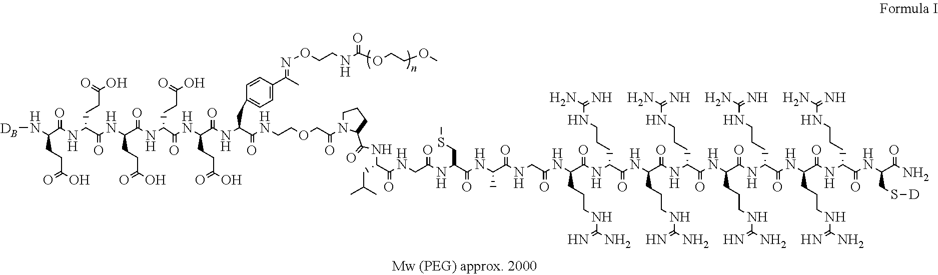

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the compound of Formula I or pharmaceutically acceptable salt thereof is administered to the subject intravenously. In some embodiments, the compound of Formula I or pharmaceutically acceptable salt thereof is administered to the subject intraperitoneally. In some embodiments, the compound of Formula I or pharmaceutically acceptable salt thereof is not administered intratumorally.

Disclosed herein, in certain embodiments, is a method of surgically excising a plurality of cancerous cells from a subject in need thereof, comprising: (i) administering to the subject an effective amount of a compound of Formula I,

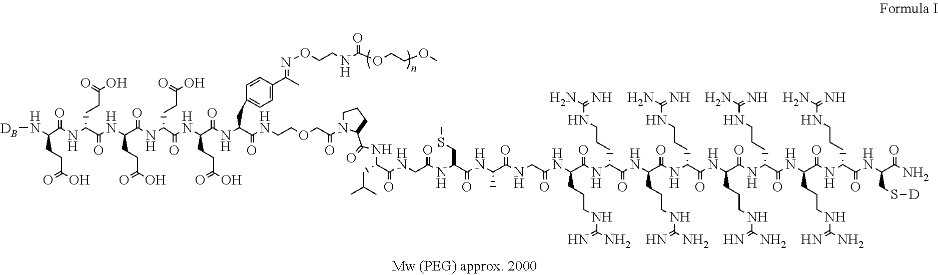

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the compound of Formula I or pharmaceutically acceptable salt thereof is administered to the subject intravenously. In some embodiments, the compound of Formula I or pharmaceutically acceptable salt thereof is administered to the subject intraperitoneally. In some embodiments, the compound of Formula I or pharmaceutically acceptable thereof is not administered intratumorally.

Disclosed herein, in certain embodiments, is a method of evaluating a tissue suspected of having a plurality of cancerous cells, comprising: (i) administering to the subject an effective amount of a compound of Formula I,

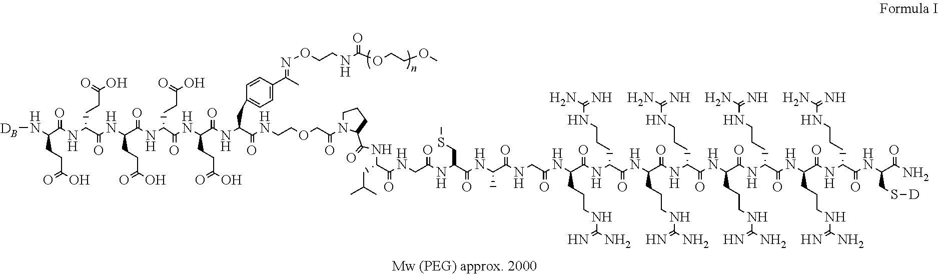

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) contacting the tissue with light having at least one excitation wavelength of the D_{A} donor moiety in Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the compound of Formula I is administered to the subject intravenously. In some embodiments, the compound of Formula I is administered to the subject intraperitoneally. In some embodiments, the compound of Formula I is not administered intratumorally.

Disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of a compound of Formula I:

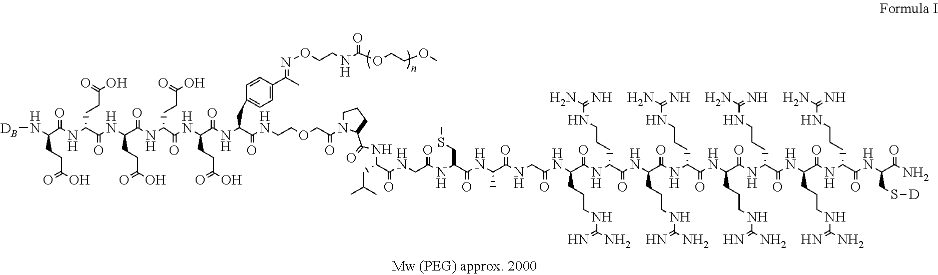

wherein: D_{A} and D_{B} are, respectively, a donor moiety and acceptor moiety that are capable of participating in Förster Resonance Energy Transfer; or a pharmaceutically acceptable salt thereof at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the compound of Formula I or pharmaceutically acceptable salt thereof is administered to the subject intravenously. In some embodiments, the compound of Formula I or pharmaceutically acceptable salt thereof is administered to the subject intraperitoneally. In some embodiments, the compound of Formula I or pharmaceutically acceptable salt thereof is not administered intratumorally.

SDM-25 is exquisitely sensitive for the tumor environment and highly stable in vivo. In humans, SDM-25 is highly stable enabling it to persist in the body for about 15 to 24 hours. Due to the design of SDM-25, the increase in Cy5 fluorescent emissions is concentrated in the tumor environment, which is characterized by high levels of proteases, such as MMPs, that cleave the substrate linker and release Cy5 from proximity to Cy7. The hydrolysis disrupts FRET causing a fluorescence color change (such as enhanced Cy5 emission and reduced Cy7 emission). The resulting fluorescence color change is long lasting in the cancer tissue due to unmasked cell penetrating peptide (CPP) properties after protease hydrolysis. The cancer tissue retention time is greater than systemic persistence of the uncleaved SDM-25. As such, SDM-25, in some embodiments, is administered to a subject via intravenous, systemic administration and detects cancers with low amounts of MMPs for up to 20 to 24 hours after administration.

Disclosed herein, in certain embodiments, is a method of surgically excising a plurality of cancerous cells from a subject in need thereof, comprising: (i) administering to the subject an effective amount of SDM-25,

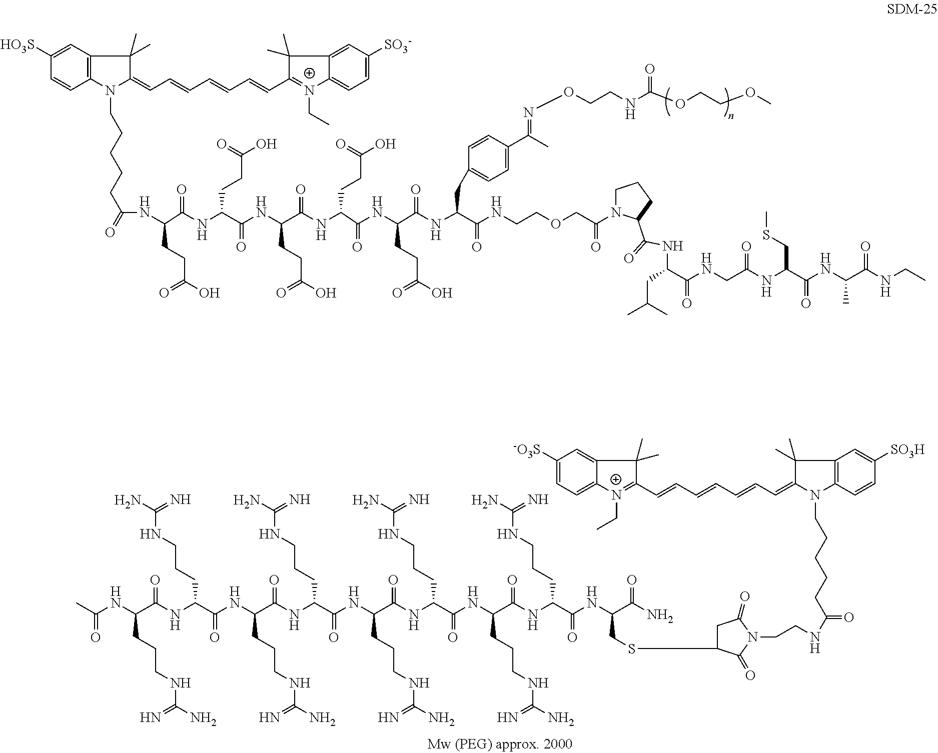

or a pharmaceutically acceptable salt thereof, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, SDM-25 is administered to the subject intravenously. In some embodiments, SDM-25 is administered to the subject intraperitoneally. In some embodiments, SDM-25 is not administered intratumorally.

Disclosed herein, in certain embodiments, is a method of surgically excising a plurality of cancerous cells from a subject in need thereof, comprising: (i) administering to the subject an effective amount of SDM-25,

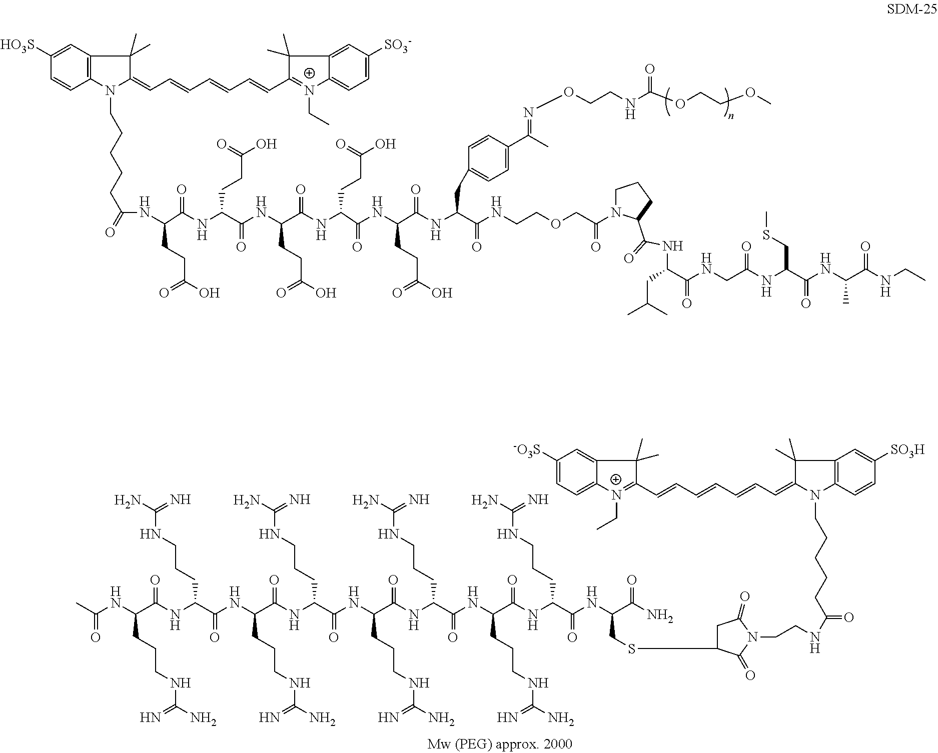

or a pharmaceutically acceptable salt thereof, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, SDM-25 or pharmaceutically acceptable salt thereof is administered to the subject intravenously. In some embodiments, the compound of Formula I is administered to the subject intraperitoneally. In some embodiments, SDM-25 or pharmaceutically acceptable salt thereof is not administered intratumorally.

Disclosed herein, in certain embodiments, is a method of evaluating a tissue suspected of having a plurality of cancerous cells, comprising: (i) administering to the subject an effective amount of SDM-25,

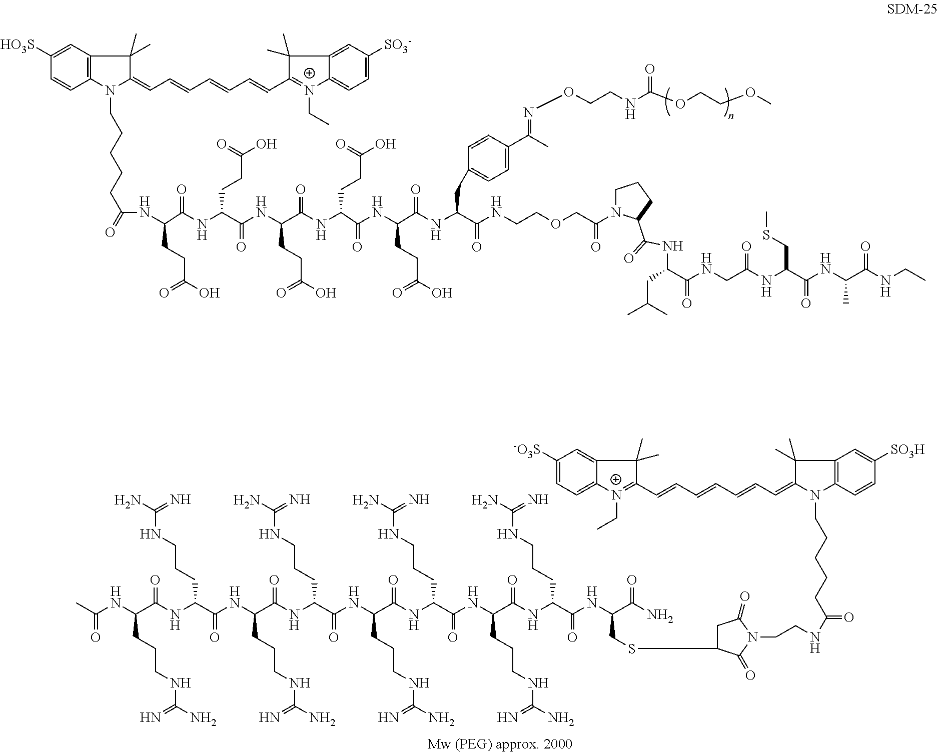

or a pharmaceutically acceptable salt thereof, at least 12 hours prior to surgery; (ii) contacting the tissue with light having at least one excitation wavelength of the Cy5 dye moiety in SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, SDM-25 is administered to the subject intravenously. In some embodiments, SDM-25 is administered to the subject intraperitoneally. In some embodiments, SDM-25 is not administered intratumorally.

Disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of SDM-25:

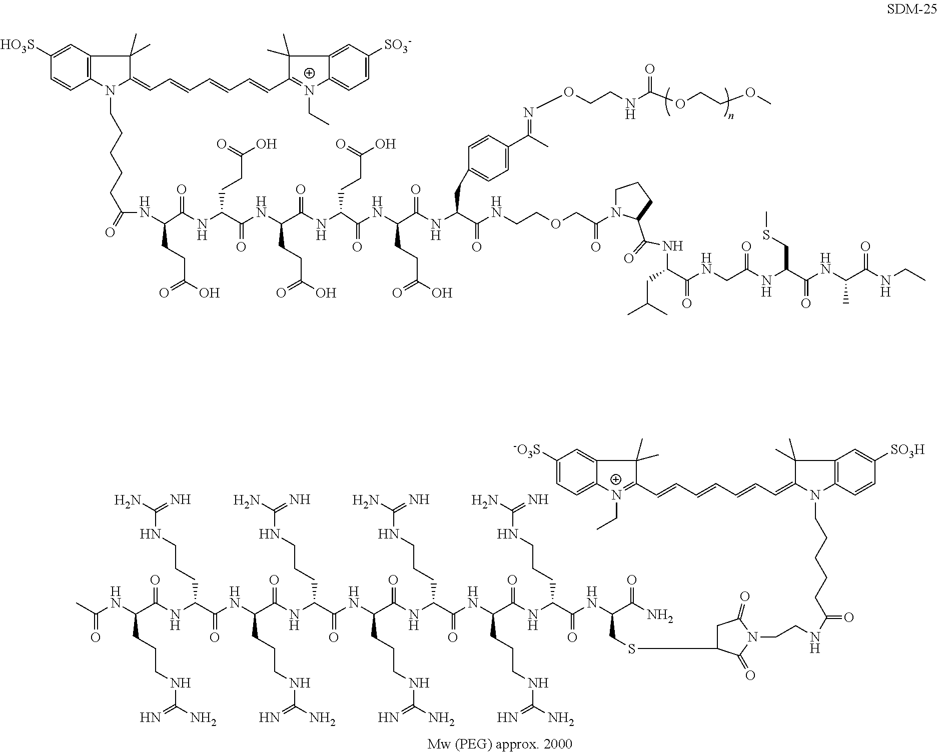

or a pharmaceutically acceptable salt thereof at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the a Cy5/Cy7 ratio value is above a determined threshold and is indicative of the presence of cancerous cells. In some embodiments, SDM-25 is administered to the subject intravenously. In some embodiments, SDM-25 is administered to the subject intraperitoneally. In some embodiments, SDM-25 is not administered intratumorally.

Pharmaceutical compositions configured as dosage forms for use in the methods described herein are also provided. Described herein, in certain embodiments, is a pharmaceutical composition configured in single dose form, wherein the single dose form comprises 1 mg to 16 mg of a compound of Formula I:

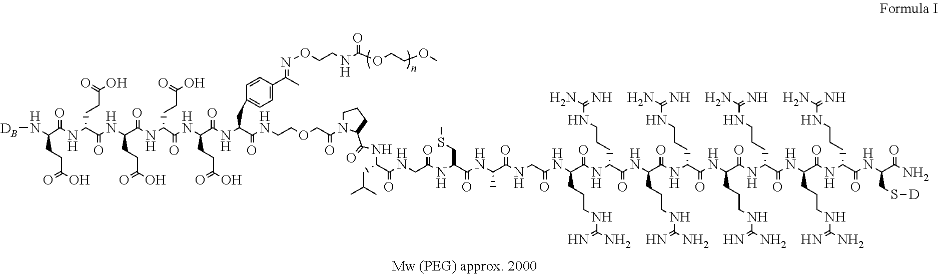

wherein: D_{A} and D_{B} are, respectively, a donor moiety and acceptor moiety that are capable of participating in Förster Resonance Energy Transfer; or a pharmaceutically acceptable salt thereof. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of a compound of Formula I:

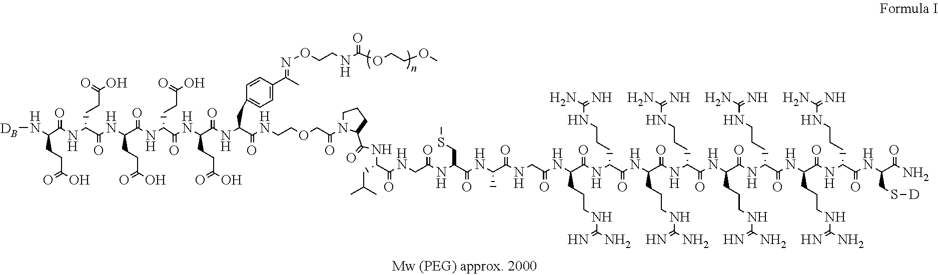

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of a compound of Formula I:

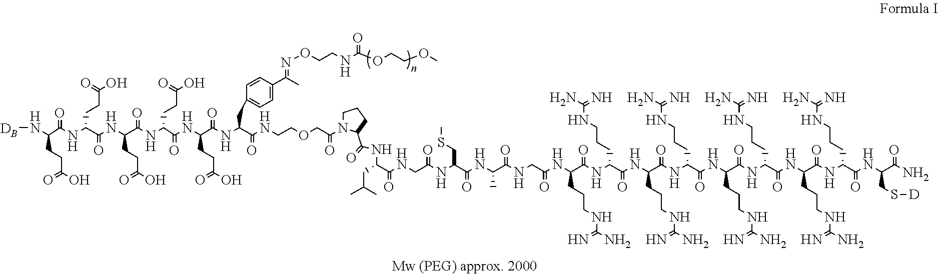

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition; at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of a compound of Formula I:

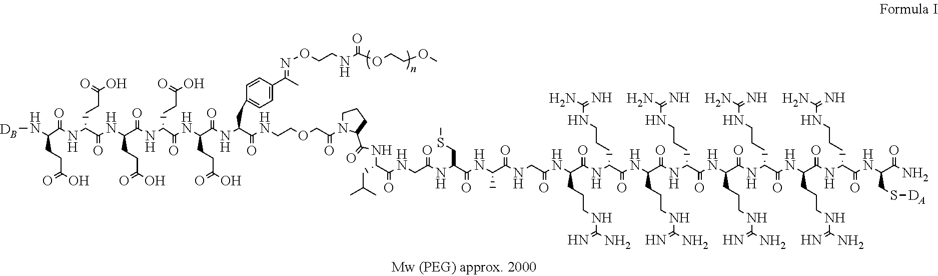

wherein: D_{A} and D_{B} are, respectively, a donor moiety and acceptor moiety that are capable of participating in Förster Resonance Energy Transfer; or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the D_{A} donor moiety in the compound of Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of a compound of Formula I:

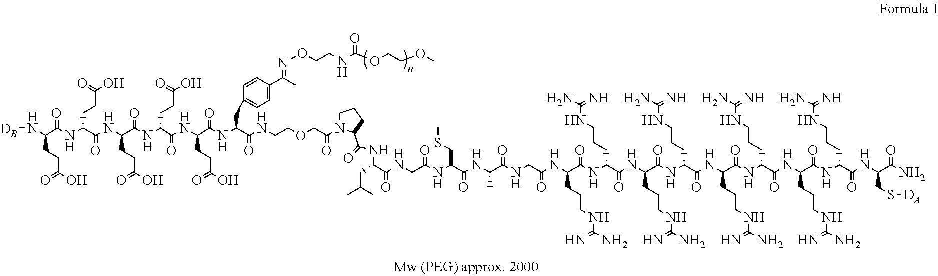

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in a single dose form, wherein the single dose form comprises 1 mg to 16 mg of a compound of SDM-25:

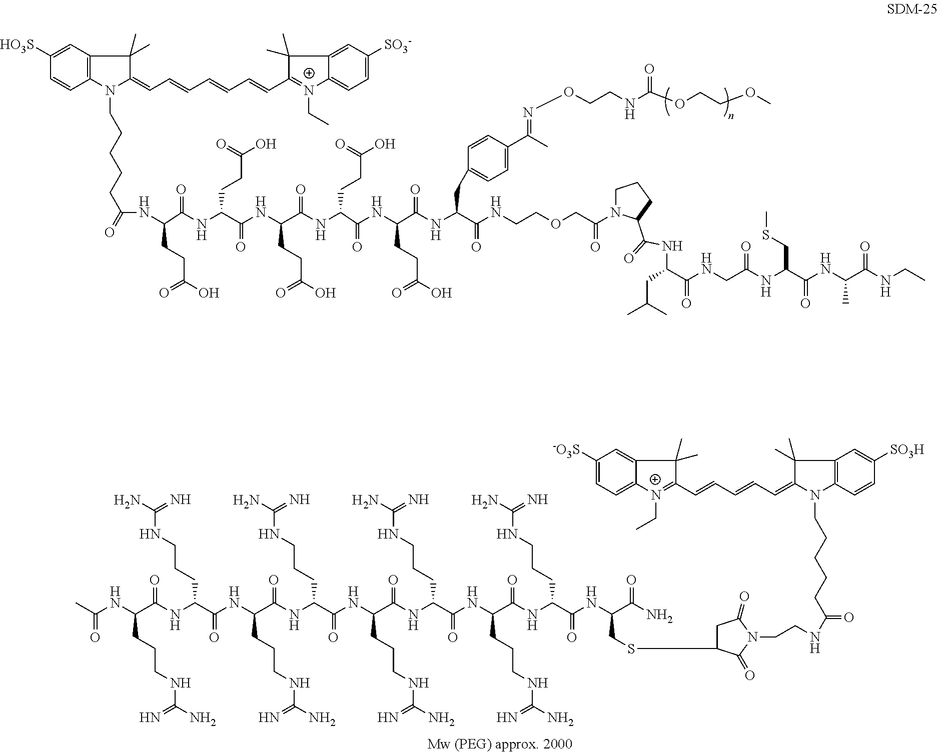

or a pharmaceutically acceptable salt thereof. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of SDM-25:

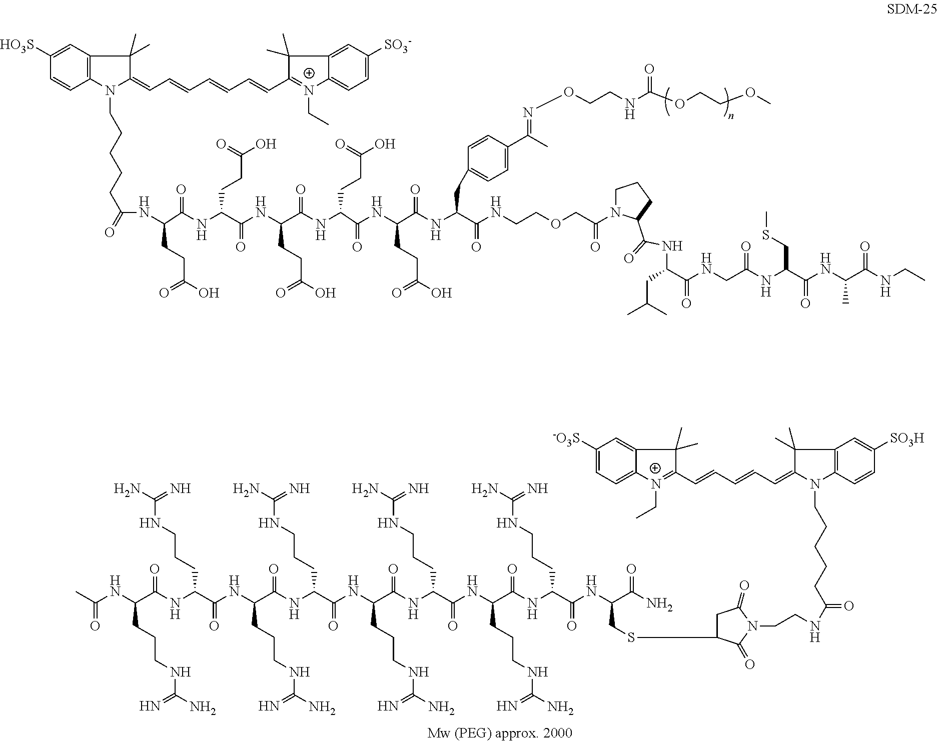

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of SDM-25:

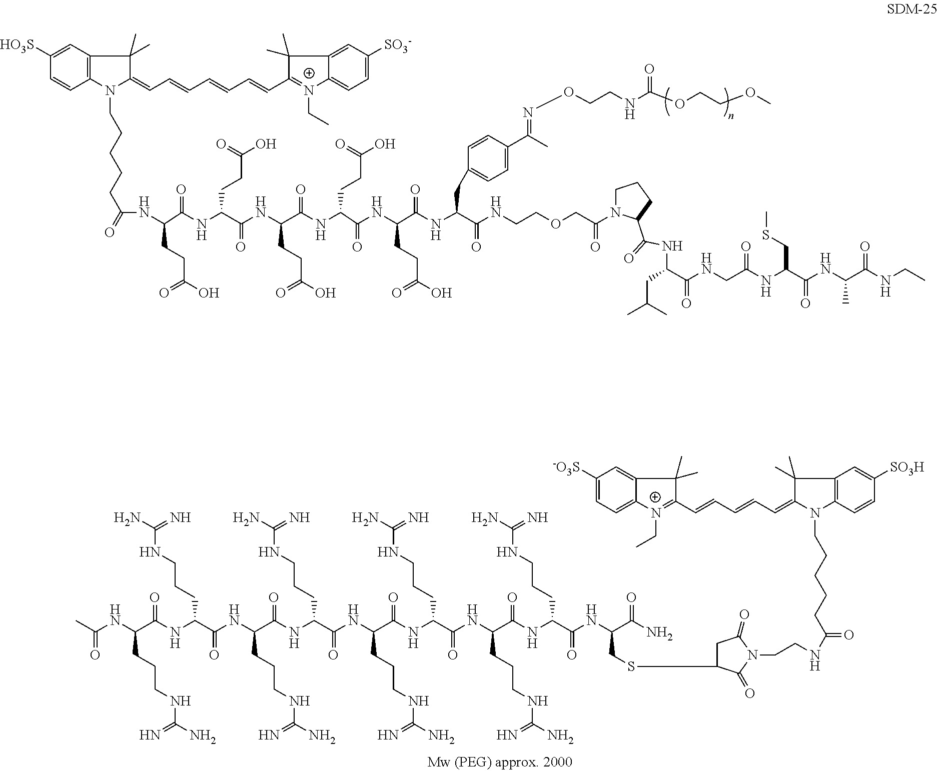

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of SDM-25:

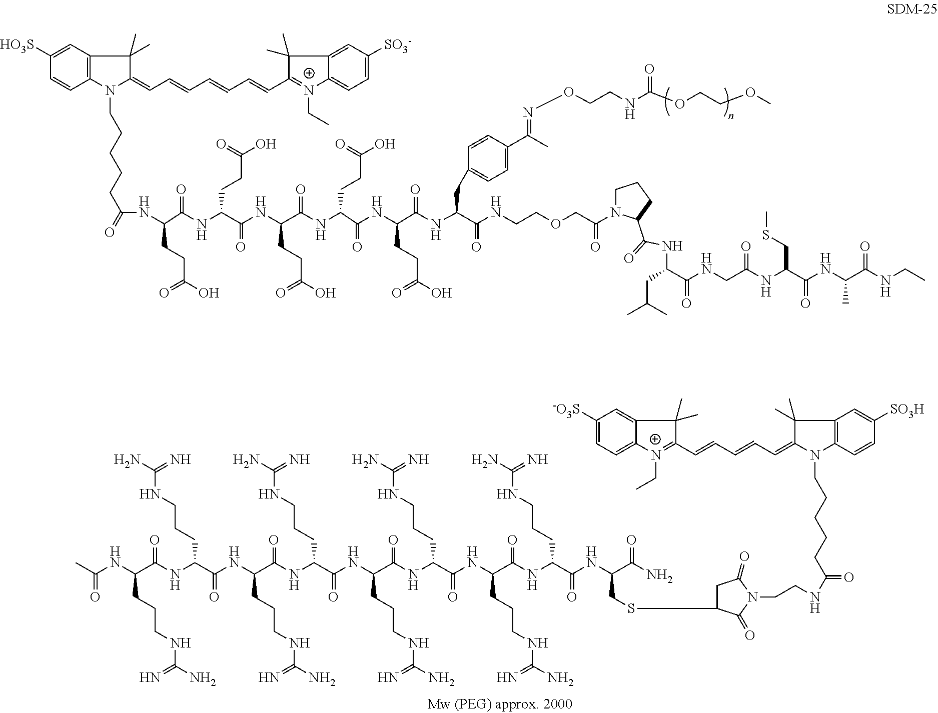

or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the Cy5 dye moiety of SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Described herein, in certain embodiments, is a pharmaceutical composition configured in single dose form, wherein the single dose form comprises 1 mg to 16 mg of SDM-25:

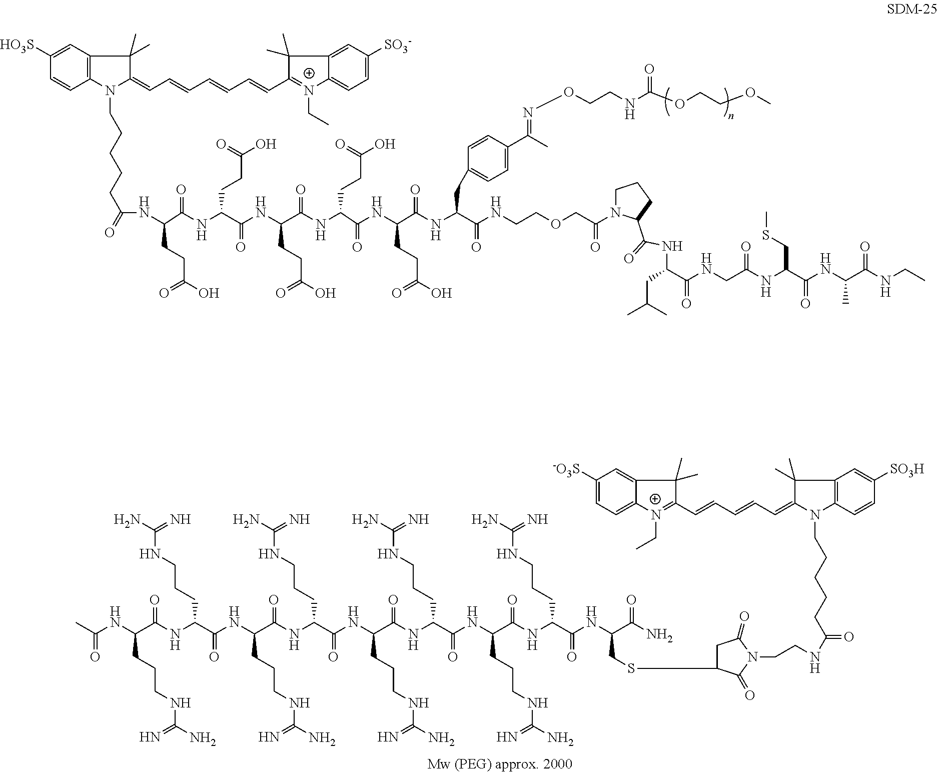

or a pharmaceutically acceptable salt thereof, for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration. In some embodiments, the single dose form is not formulated for intratumoral administration.

Formulations of SDMs described herein (e.g., a compound of Formula I, e.g., SDM-25) suitable for parenteral injection, e.g., intravenous or intraperitoneal injection, include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants.

SDMs described herein (e.g., a compound of Formula I, e.g., SDM-25) is optionally formulated in aqueous solutions, preferably in physiologically compatible buffers such as Hank's solution, Ringer's solution, or physiological saline buffer. Formulations of SDM-25 for use with the methods disclosed herein include aqueous solutions of SDM-25 in water soluble form.

### Cancers

Described herein, in certain embodiments, is a method of surgically excising a plurality of cancerous cells from a subject in need thereof, comprising: (i) administering to the subject an effective amount of a compound of Formula I,

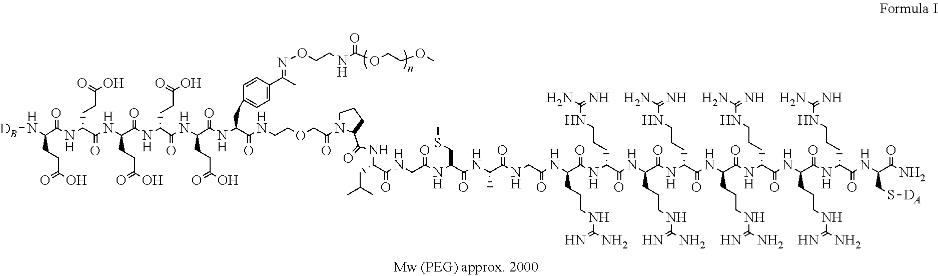

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Disclosed herein, in certain embodiments, is a method of surgically excising a plurality of cancerous cells from a subject in need thereof, comprising: (i) administering to the subject an effective amount of SDM-25,

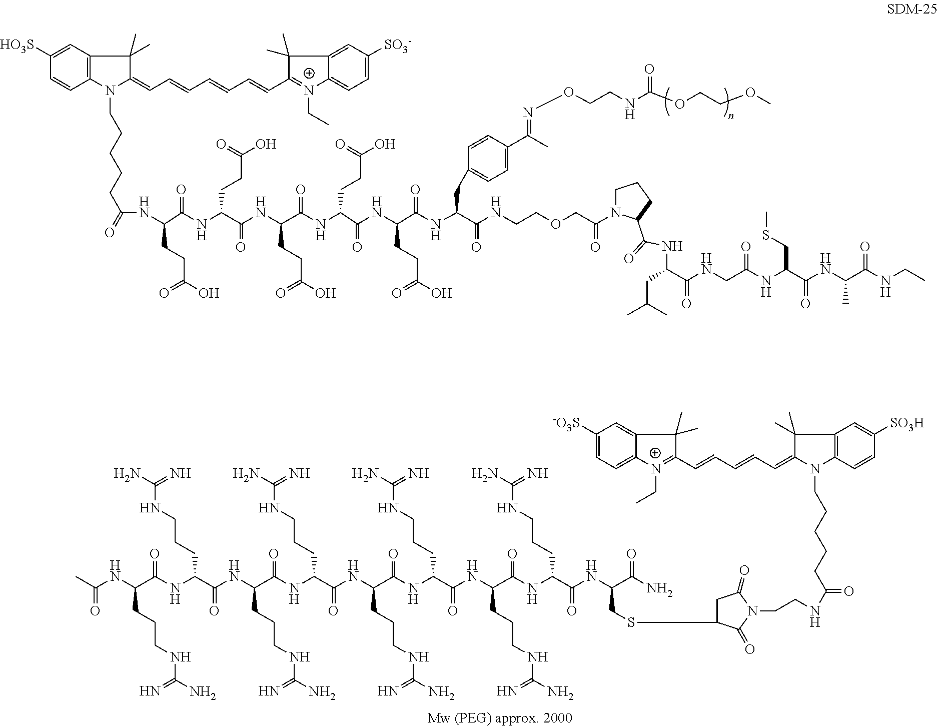

or a pharmaceutically acceptable salt thereof, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Disclosed herein, in certain embodiments, is a method of surgically excising a plurality of cancerous cells from a subject in need thereof, comprising: (i) administering to the subject an effective amount of a compound of Formula I,

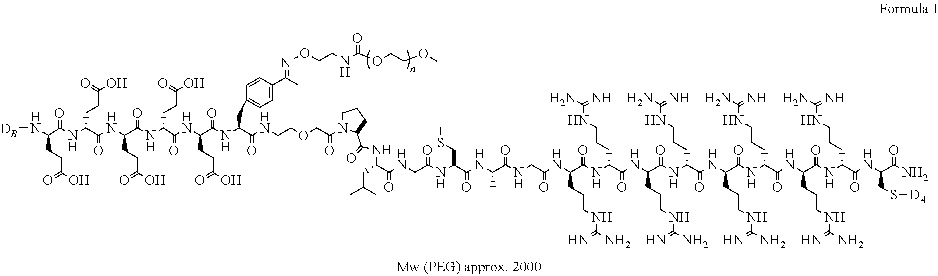

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells.

Disclosed herein, in certain embodiments, is a method of surgically excising a plurality of cancerous cells from a subject in need thereof, comprising: (i) administering to the subject an effective amount of SDM-25,

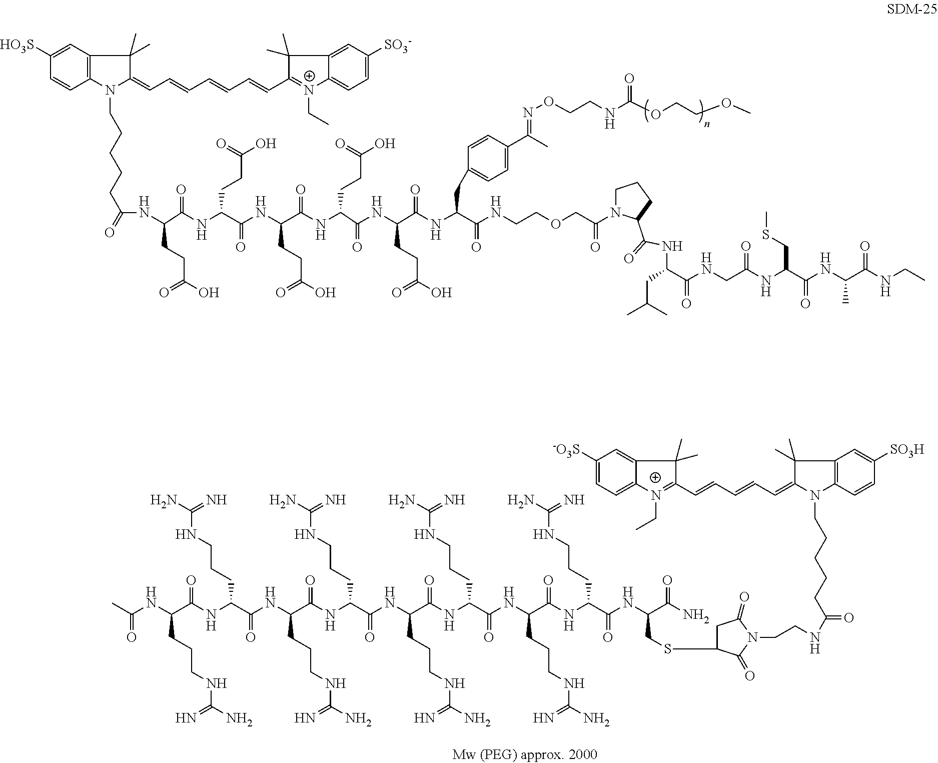

or a pharmaceutically acceptable salt thereof, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells.

Disclosed herein, in certain embodiments, is a method of evaluating a tissue suspected of having a plurality of cancerous cells, comprising: (i) administering to the subject an effective amount of a compound of Formula I,

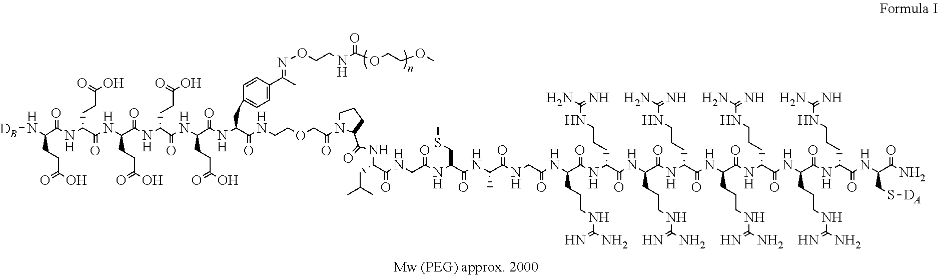

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) contacting the tissue with light having at least one excitation wavelength of the D_{A} donor moiety in Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value, thereby determining that the tissue comprises a plurality of cancerous cells; or not detecting said region and thereby determining that the tissue does not comprise a plurality of cancerous cells.

Disclosed herein, in certain embodiments, is a method of evaluating a tissue suspected of having a plurality of cancerous cells, comprising: (i) administering to the subject an effective amount of SDM-25,

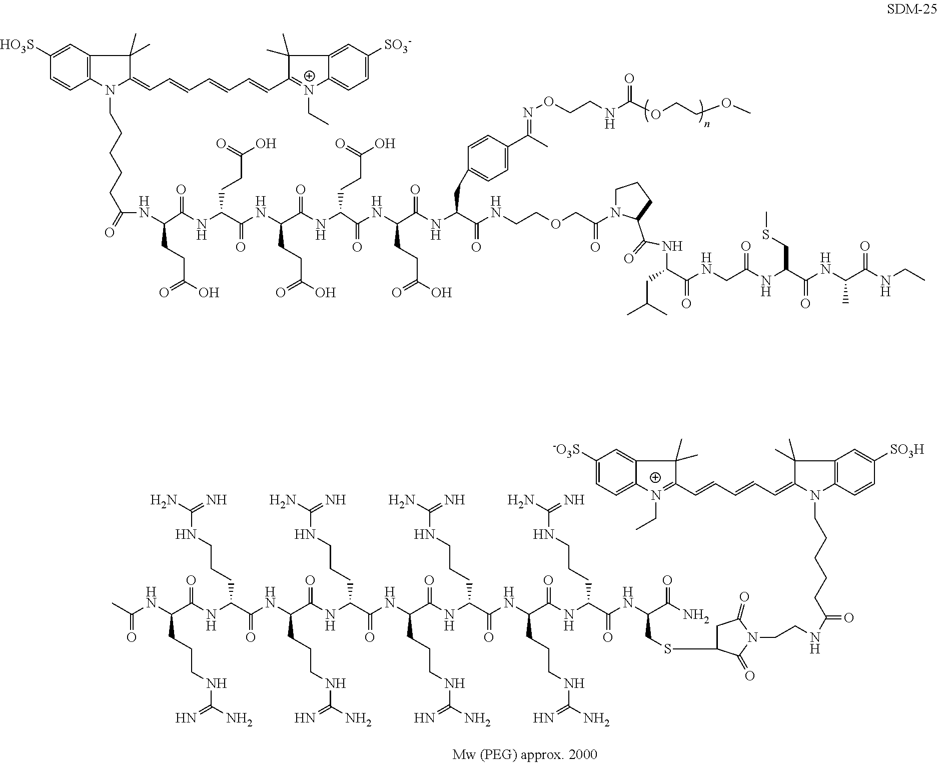

or a pharmaceutically acceptable salt thereof, at least 12 hours prior to surgery; (ii) contacting the tissue with light having at least one excitation wavelength of the Cy5 dye moiety in SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells.

Disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of a compound of Formula I:

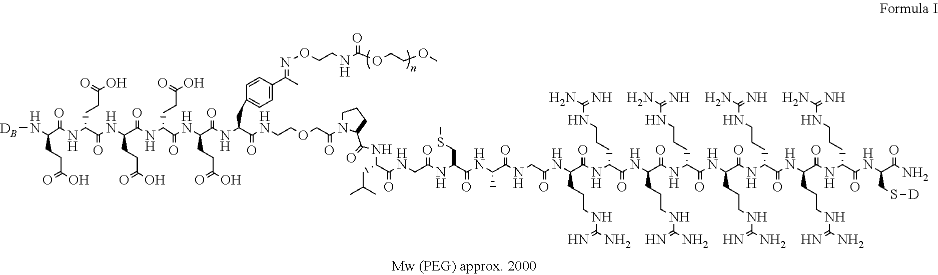

wherein: D_{A} and D_{B} are, respectively, a donor moiety and acceptor moiety that are capable of participating in Förster Resonance Energy Transfer; or a pharmaceutically acceptable salt thereof at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the a D_{A}/D_{B} ratio value is above a determined threshold and is indicative of the presence of cancerous cells.

Disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of SDM-25:

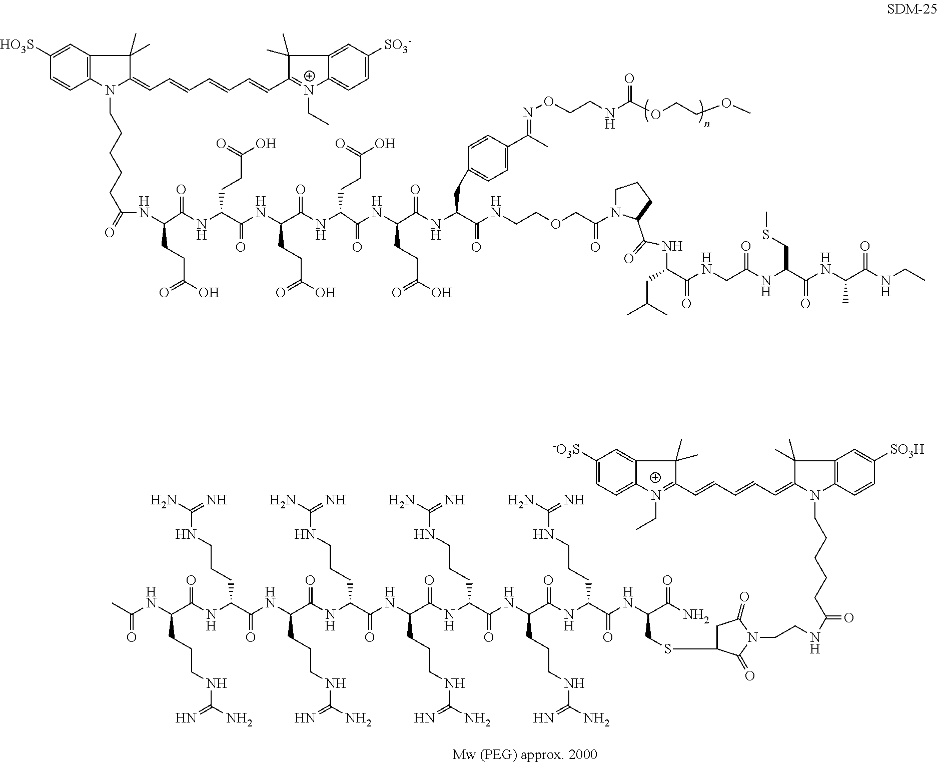

or a pharmaceutically acceptable salt thereof at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the a Cy5/Cy7 ratio value ratio is above a determined threshold and is indicative of the presence of cancerous cells.

Pharmaceutical compositions configured as dosage forms for use in the methods described herein are also provided. Described herein, in certain embodiments, is a pharmaceutical composition configured in single dose form, wherein the single dose form comprises 1 mg to 16 mg of a compound of Formula I:

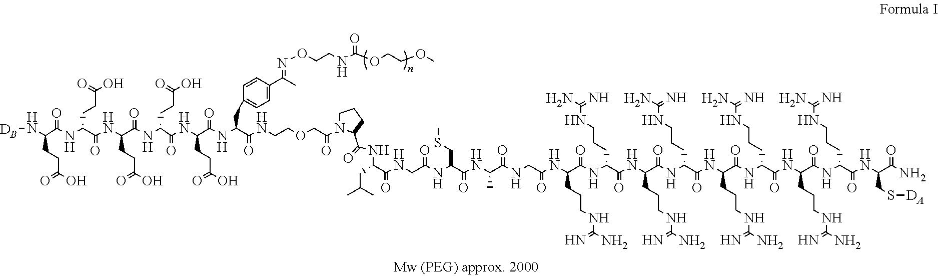

wherein: D_{A} and D_{B} are, respectively, a donor moiety and acceptor moiety that are capable of participating in Förster Resonance Energy Transfer; or a pharmaceutically acceptable salt thereof. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

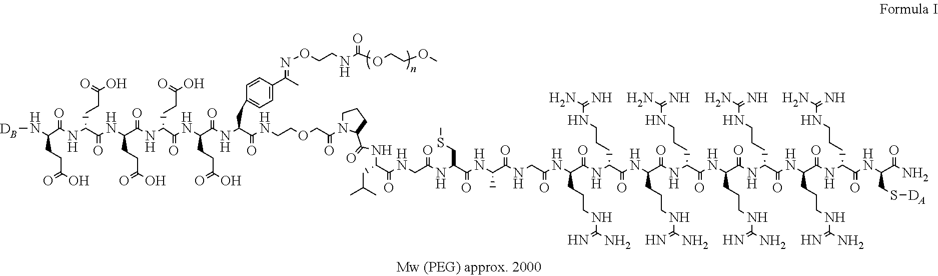

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

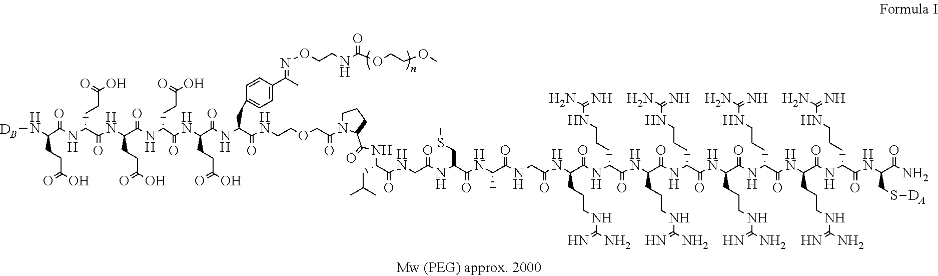

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition; at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

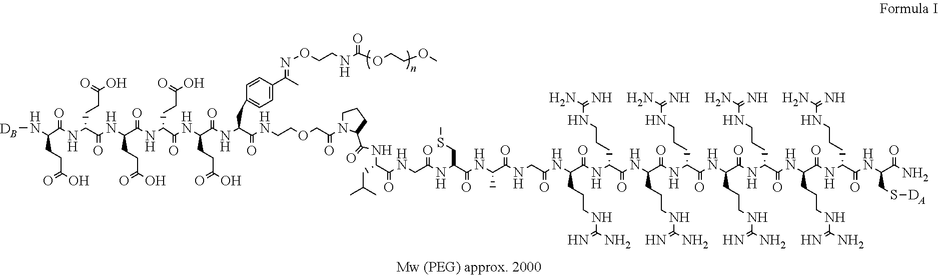

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the D_{A} donor moiety in the compound of Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

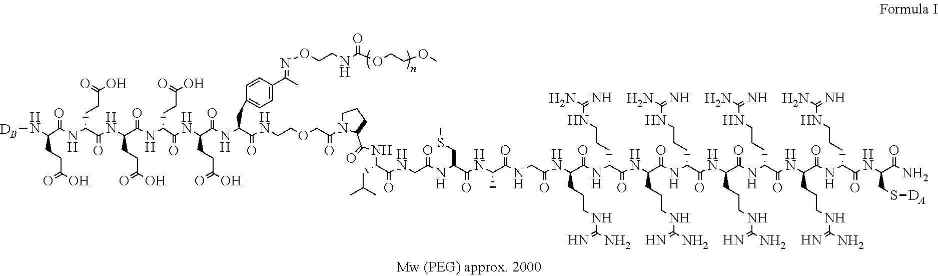

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in a single dose form, wherein the single dose form comprises 1 mg to 16 mg of a compound of SDM-25:

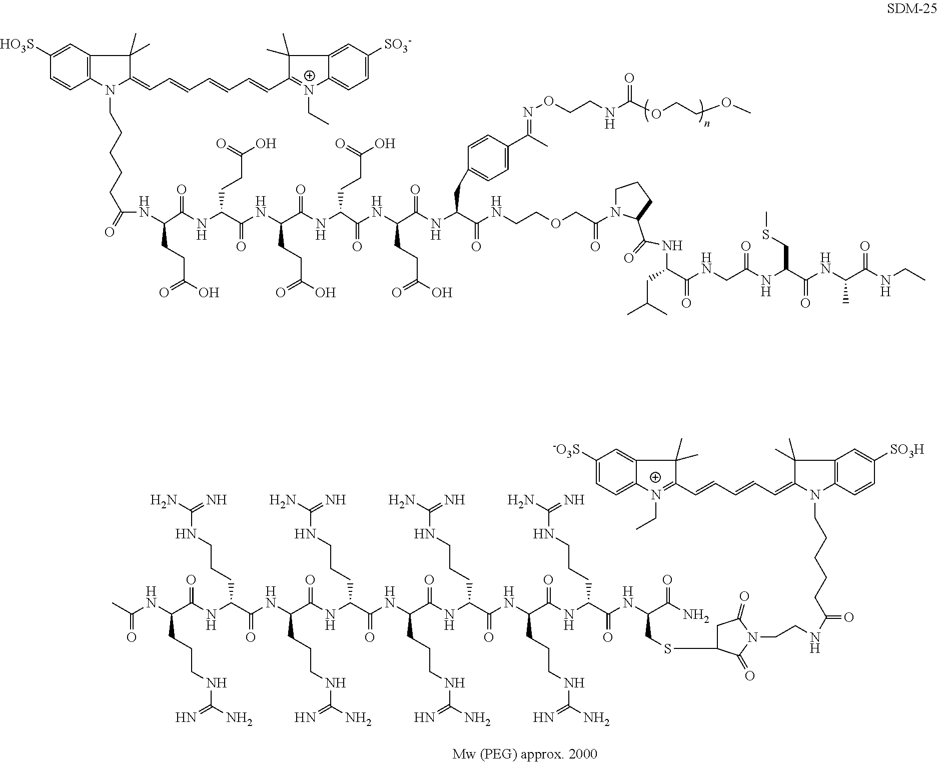

or a pharmaceutically acceptable salt thereof. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

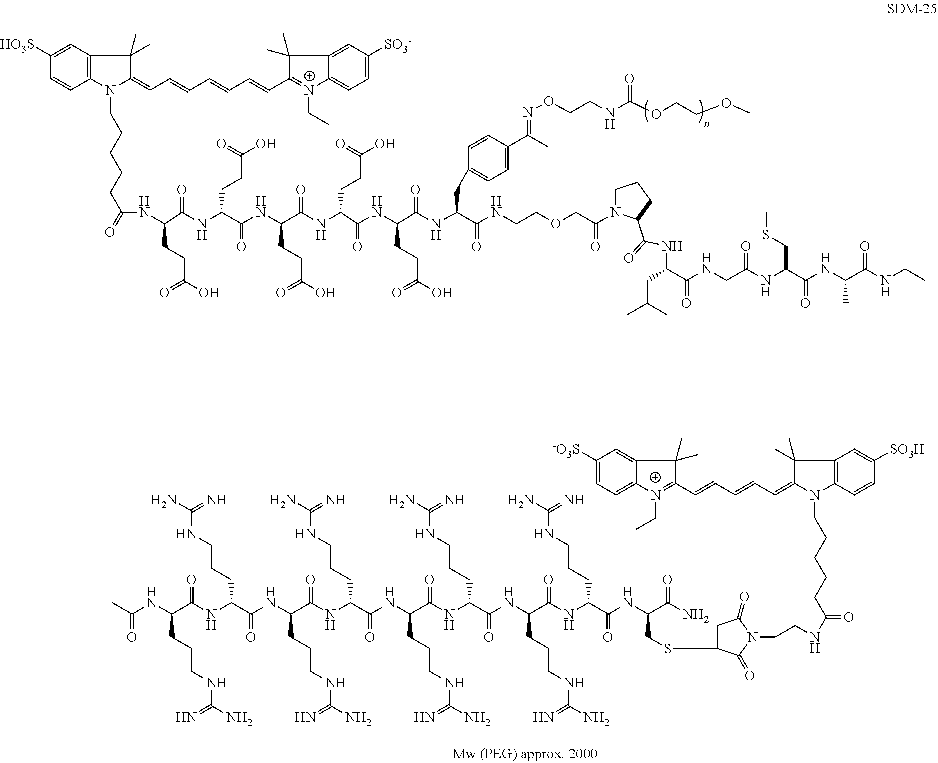

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

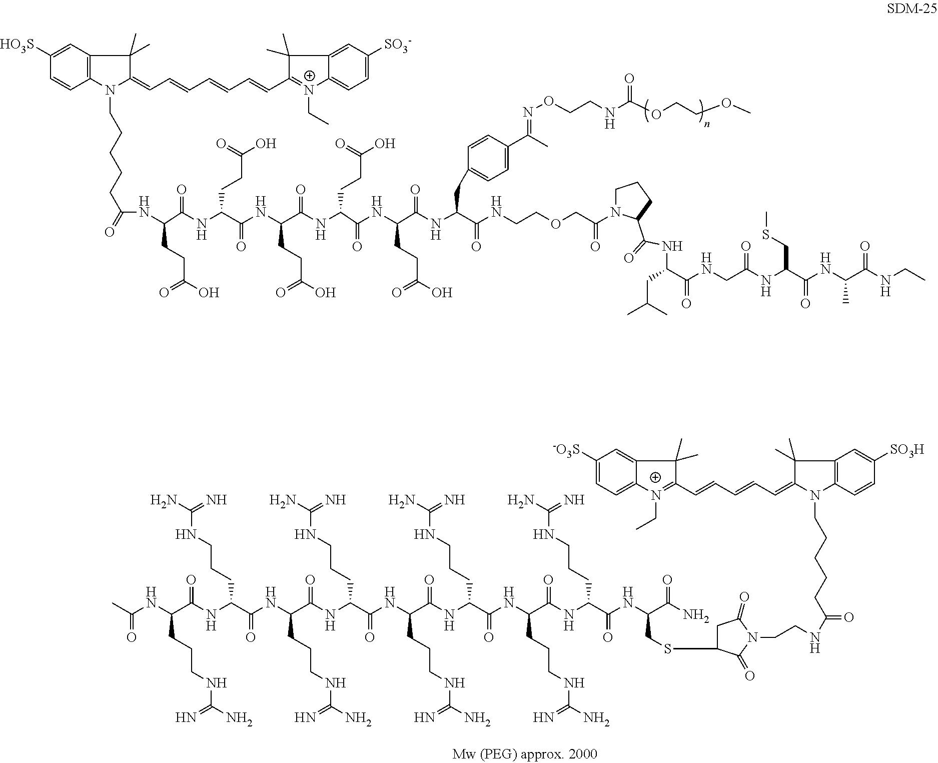

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

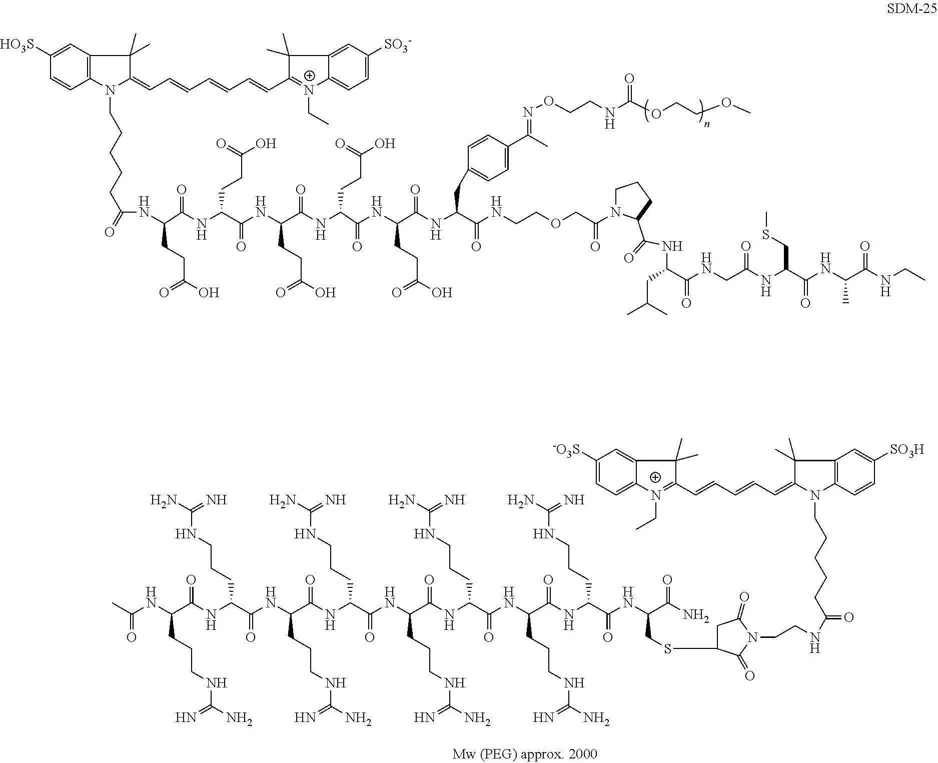

or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the Cy5 dye moiety of SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

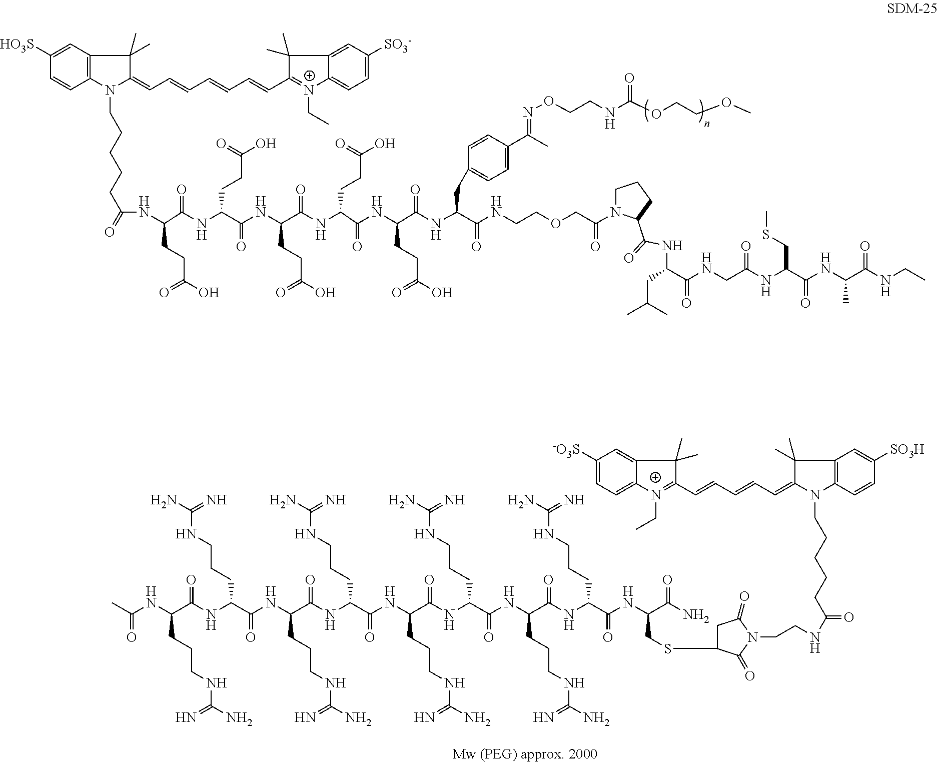

or a pharmaceutically acceptable salt thereof, for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

SDMs described herein, e.g., a compound of Formula I, e.g., SDM-25, are suitable for use in identifying cancer cells associated with a variety of solid tumors, including but not limited to breast, ovarian, colorectal, sarcoma, head and neck, prostate solid tumors. The same MMPs that cleave and activate SDM-25 in breast cancer patients are generally found in a variety of solid tumors. In some embodiments, a plurality of a cancer cells described herein is of a cancer selected from the group consisting of AIDS-related cancers (e.g., AIDS-related lymphoma), anal cancer, basal cell carcinoma, bile duct cancer (e.g., extrahepatic), bladder cancer, bone cancer, (e.g., osteosarcoma and malignant fibrous histiocytoma), breast cancer, cervical cancer, colon cancer, colorectal cancer, endometrial cancer (e.g., uterine cancer), ependymoma, esophageal cancer, eye cancer (e.g., intraocular melanoma and retinoblastoma), gastric (stomach) cancer, germ cell tumor, (e.g., extracranial, extragonadal, ovarian), head and neck cancer, leukemia, lip and oral cavity cancer, liver cancer, lung cancer (e.g., small cell lung cancer, non-small cell lung cancer, adenocarcinoma of the lung, and squamous carcinoma of the lung), melanoma, Merkel cell carcinoma, neuroendocrine cancer, ovarian cancer, pancreatic cancer, penile cancer, pituitary tumor, prostate cancer, renal cancer, salivary gland cancers, sarcoma, skin cancer, small intestine cancer, squamous cell cancer, testicular cancer, throat cancer, thyroid cancer, urethral cancer, vaginal cancer, and post-transplant lymphoproliferative disorder (PTLD).

In some embodiments, plurality of a cancer cells is of a cancer selected from the group consisting of breast cancer, colorectal cancer, melanoma, sarcoma, head and neck carcinoma, ovarian cancer, and prostate cancer.

In some embodiments, the plurality of cancerous cells is of breast cancer. In some embodiments, the breast cancer is a mixed breast cancer, wherein the mixed breast cancer comprises invasive and non-invasive breast cancer. In some embodiments, the breast cancer is an invasive breast cancer. In some embodiments, the breast cancer is a non-invasive breast cancer. In some embodiments, the non-invasive breast cancer is ductal carcinoma in situ.

In some embodiments, the plurality of cancerous cells is of head-and-neck carcinoma. In some embodiments, the plurality of cancerous cells is of colorectal cancer. In some embodiments, the plurality of cancerous cells is of ovarian cancer. In some embodiments, the plurality of cancerous cells is of prostate cancer.

### Fluorescence Detection

Ratiometric fluorescence detection and single intensity fluorescence detection are methodologies for use in in vitro and in vivo imaging applications, which include the methods described herein.

Described herein, in certain embodiments, is a method of surgically excising a plurality of cancerous cells from a subject in need thereof, comprising: (i) administering to the subject an effective amount of a compound of Formula I,

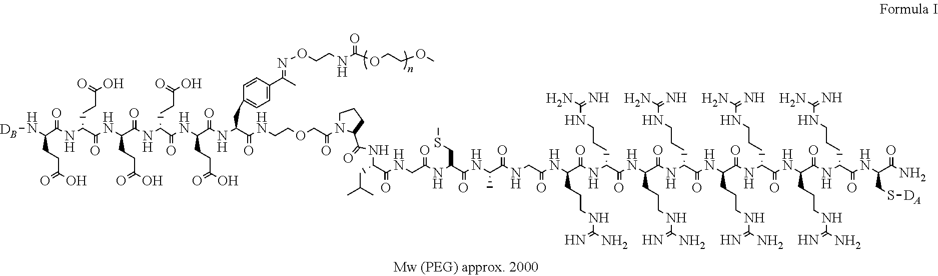

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Disclosed herein, in certain embodiments, is a method of surgically excising a plurality of cancerous cells from a subject in need thereof, comprising: (i) administering to the subject an effective amount of SDM-25,

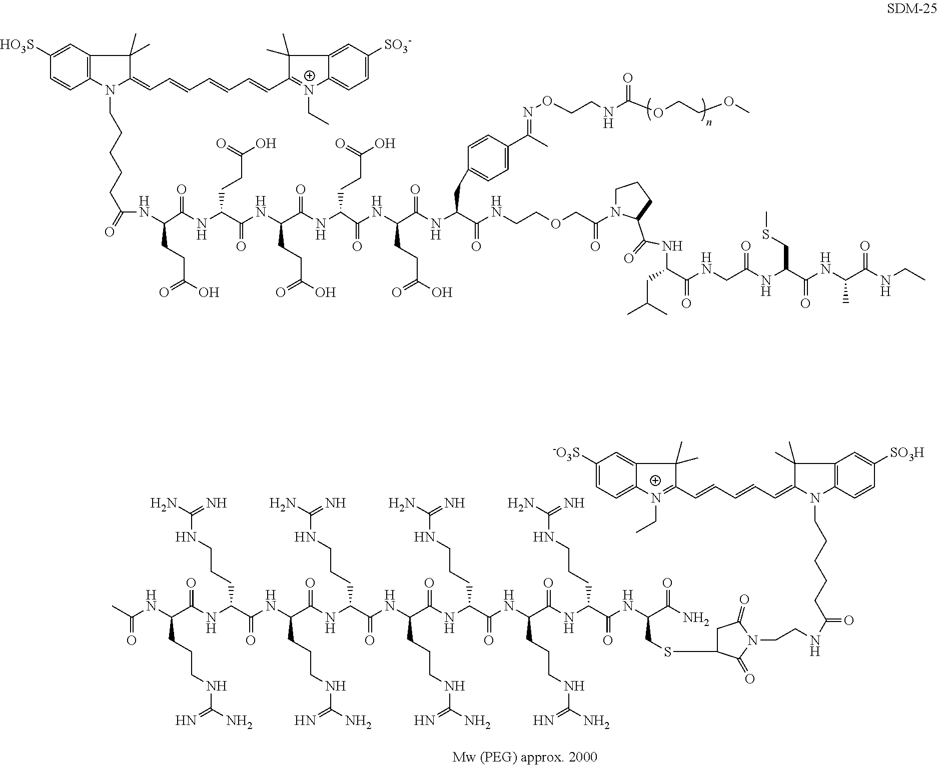

or a pharmaceutically acceptable salt thereof, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject.

Disclosed herein, in certain embodiments, is a method of surgically excising a plurality of cancerous cells from a subject in need thereof, comprising: (i) administering to the subject an effective amount of a compound of Formula I,

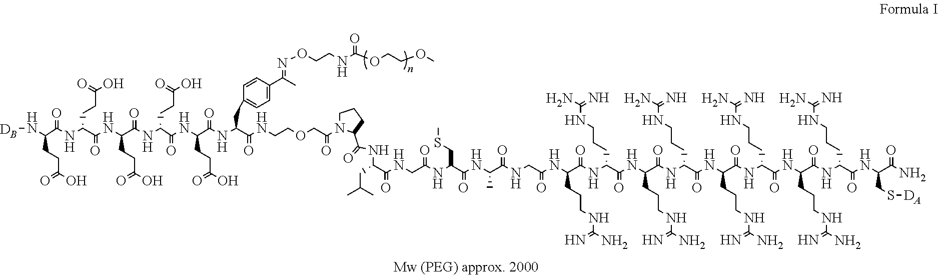

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells.

Disclosed herein, in certain embodiments, is a method of surgically excising a plurality of cancerous cells from a subject in need thereof, comprising: (i) administering to the subject an effective amount of SDM-25,

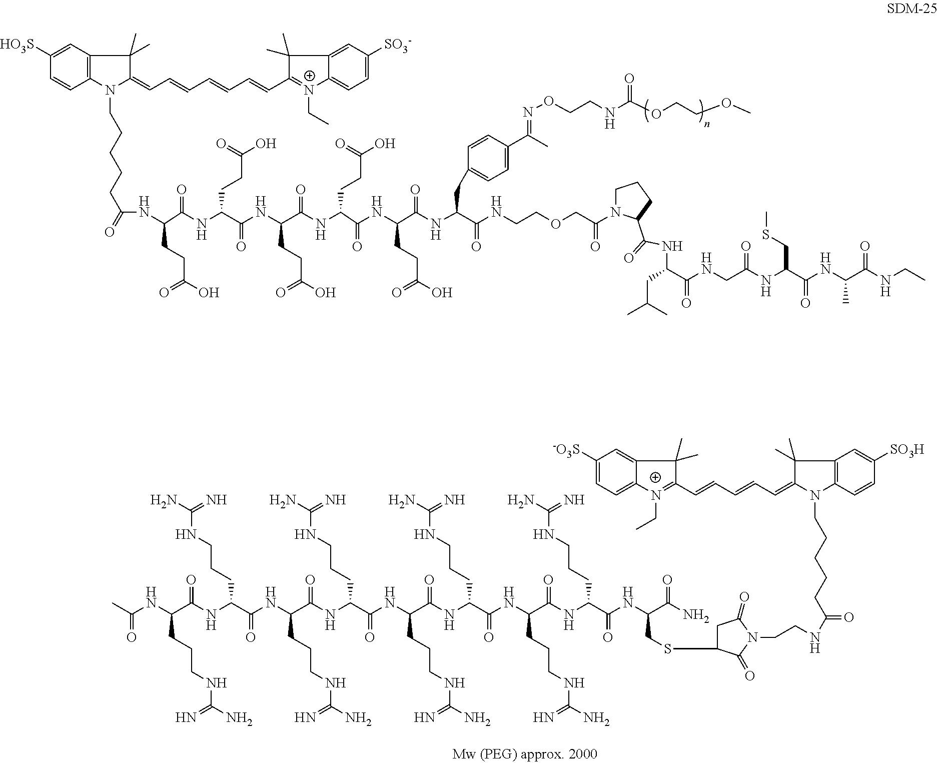

or a pharmaceutically acceptable salt thereof, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells.

Disclosed herein, in certain embodiments, is a method of evaluating a tissue suspected of having a plurality of cancerous cells, comprising: (i) administering to the subject an effective amount of a compound of Formula I,

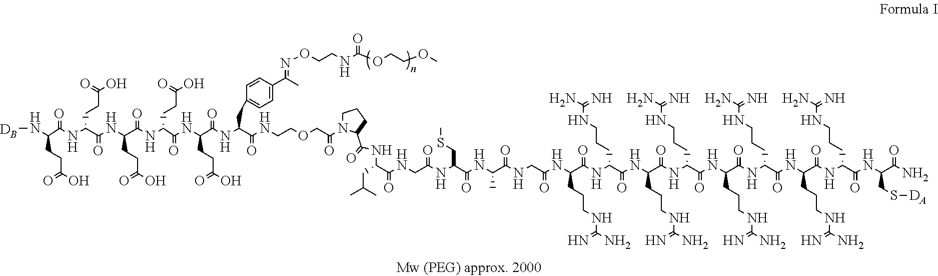

wherein: D_{A} and D_{B} are, respectively, a donor moiety and acceptor moiety that are capable of participating in Förster Resonance Energy Transfer; or a pharmaceutically acceptable salt thereof, at least 12 hours prior to surgery; (ii) contacting the tissue with light having at least one excitation wavelength of the D_{A} donor moiety in Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value, thereby determining that the tissue comprises a plurality of cancerous cells; or not detecting said region and thereby determining that the tissue does not comprise a plurality of cancerous cells.

Disclosed herein, in certain embodiments, is a method of evaluating a tissue suspected of having a plurality of cancerous cells, comprising: (i) administering to the subject an effective amount of SDM-25,

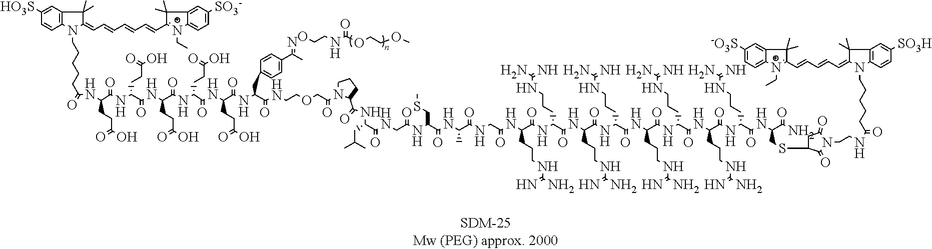

or a pharmaceutically acceptable salt thereof, at least 12 hours prior to surgery; (ii) contacting the tissue with light having at least one excitation wavelength of the Cy5 dye moiety in SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells.

Disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of a compound of Formula I:

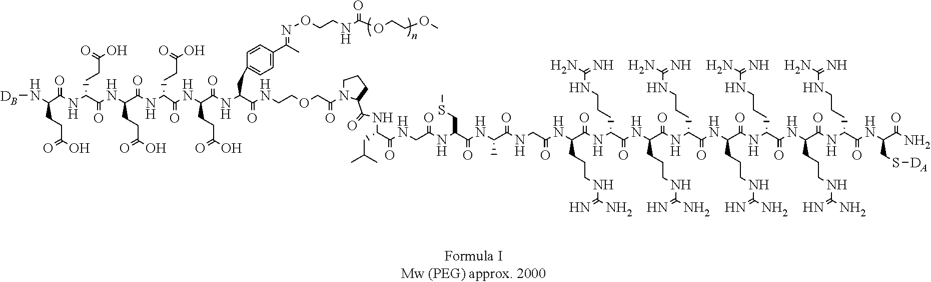

wherein: D_{A} and D_{B} are, respectively, a donor moiety and acceptor moiety that are capable of participating in Förster Resonance Energy Transfer; or a pharmaceutically acceptable salt thereof at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells.

Disclosed herein, in certain embodiments, is a method of imaging tissue of a subject known or suspected to contain cancerous cells either at a tissue site in vivo or excised therefrom, said method comprising: (i) intravenously or intraperitoneally administering to the subject an effective amount of SDM-25:

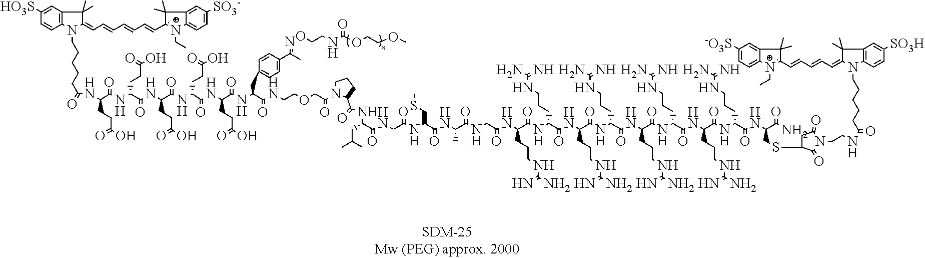

or a pharmaceutically acceptable salt thereof at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the a Cy5/Cy7 ratio value is above a determined threshold and is indicative of the presence of cancerous cells.

Pharmaceutical compositions configured as dosage forms for use in the methods described herein are also provided. Described herein, in certain embodiments, is a pharmaceutical composition configured in single dose form, wherein the single dose form comprises 1 mg to 16 mg of a compound of Formula I:

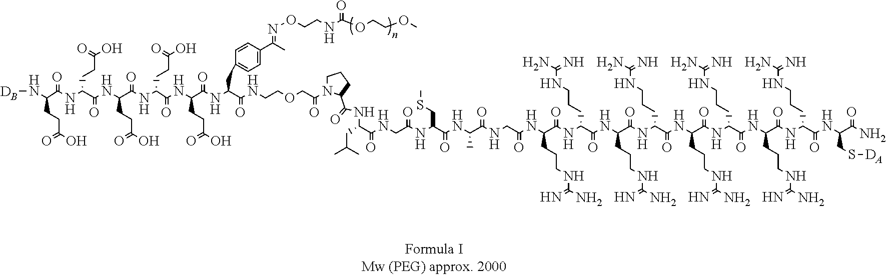

wherein: D_{A} and D_{B} are, respectively, a donor moiety and acceptor moiety that are capable of participating in Förster Resonance Energy Transfer; or a pharmaceutically acceptable salt thereof. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

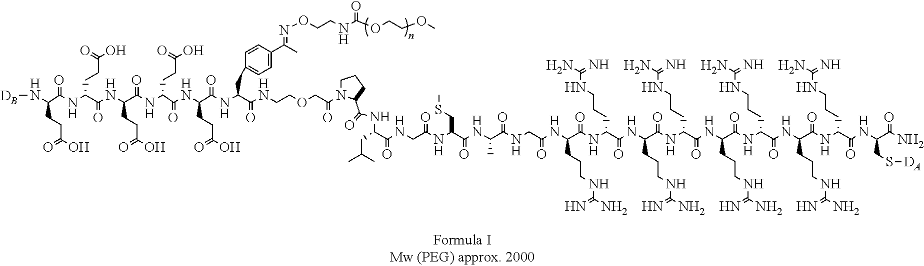

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value; or detecting a region of the image of D_{B} dye moiety fluorescence intensity in which the image has a D_{B} acceptor moiety fluorescence intensity above a pre-specified fluorescence intensity threshold value; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

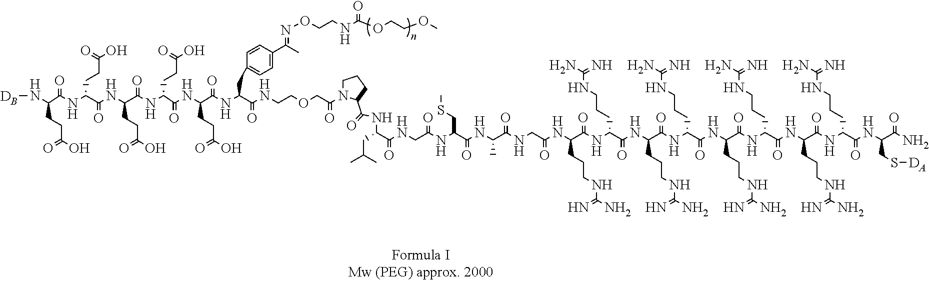

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for surgically excising a plurality of cancerous cells from a subject in need thereof, comprising: (i) administering to the subject an effective amount of the composition; at least 12 hours prior to surgery; (ii) surgically excising a tissue from the subject to yield a surgical cavity; (iii) contacting the excised tissue with light having at least one excitation wavelength of the D_{A} donor moiety of the compound of Formula I; (iv) capturing an image of D_{A} donor moiety fluorescence intensity in the excised tissue and, optionally, capturing an image of D_{B} acceptor moiety fluorescence intensity in the excised tissue; (v) detecting a region of the image of D_{A} donor moiety fluorescence intensity in which the image has a D_{A} donor moiety fluorescence intensity above a pre-specified fluorescence intensity threshold value up to 2 mm of a margin of the excised tissue; or detecting a region of the image of D_{B} dye moiety fluorescence intensity in which the image has a D_{B} acceptor moiety fluorescence intensity above a pre-specified fluorescence intensity threshold value up to 2 mm of a margin of the excised tissue; or detecting a region of a fluorescence ratio image of D_{A} donor moiety fluorescence intensity to D_{B} acceptor moiety fluorescence intensity, in which the ratio image has a D_{A}/D_{B} ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

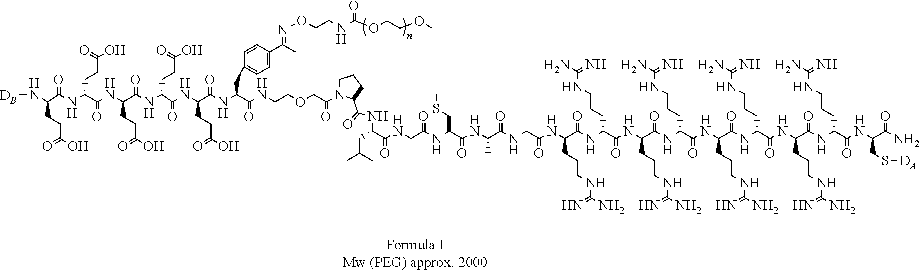

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the D_{A} donor moiety in the compound of Formula I; (iii) capturing an image of D_{A} donor moiety fluorescence intensity in the excised tissue and, optionally, capturing an image of D_{B} acceptor moiety fluorescence intensity in the tissue; (iv) detecting a region of the image of D_{A} donor moiety fluorescence intensity in which the image has a D_{A} donor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of D_{B} dye moiety fluorescence intensity in which the image has a D_{B} acceptor moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of D_{A} donor moiety fluorescence intensity to D_{B} acceptor moiety fluorescence intensity, in which the ratio image has D_{A}/D_{B} ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of a compound of Formula I:

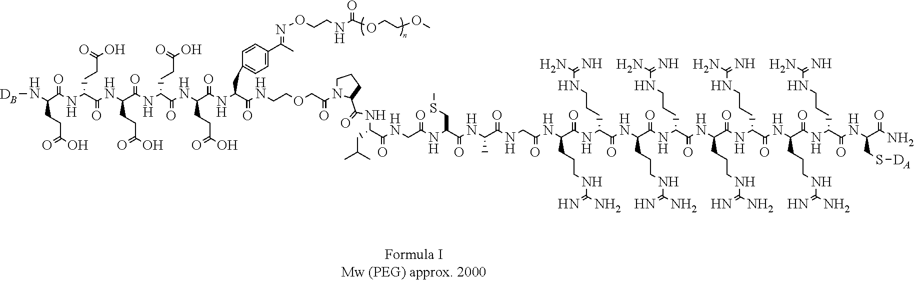

wherein: D_{A} and D_{B} are, respectively, a donor moiety and acceptor moiety that are capable of participating in Förster Resonance Energy Transfer; or a pharmaceutically acceptable salt thereof; for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby the compound of Formula I is taken up by cells at the tissue site; (ii) contacting tissue at said tissue site or excised therefrom with light having at least one excitation wavelength of the D_{A} donor moiety of the compound of Formula I; (iii) capturing an image of D_{A} donor moiety fluorescence intensity in the tissue and, optionally, capturing an image of D_{B} acceptor moiety fluorescence intensity in the tissue; and (iv) detecting a region of the image of D_{A} donor moiety fluorescence intensity whereby the fluorescence intensity is above a determined threshold and is indicative of the presence of cancerous cells; or detecting a region of the image of D_{B} acceptor moiety fluorescence intensity whereby the fluorescence intensity is above a determined threshold and is indicative of the presence of cancerous cells; or detecting a region of a fluorescence ratio image of D_{A} donor moiety fluorescence intensity to D_{B} acceptor moiety fluorescence intensity, whereby the D_{A}/D_{B} ratio value is above a determined threshold and is indicative of the presence of cancerous cells. In some embodiments, the single dose form comprises 8 mg of the compound of Formula I or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in a single dose form, wherein the single dose form comprises 1 mg to 16 mg of a compound of SDM-25:

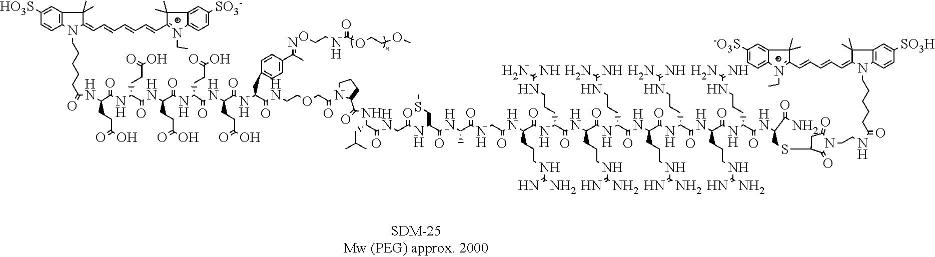

or a pharmaceutically acceptable salt thereof. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

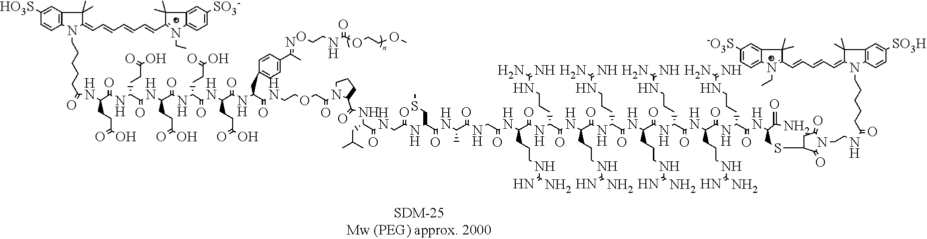

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) during surgery, contacting a tissue suspected to comprise a plurality of cancerous cells with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value; or detecting a region of the image of Cy7 dye moiety fluorescence intensity in which the image has a Cy7 dye moiety fluorescence intensity above a pre-specified fluorescence intensity threshold value; or detecting a region of a fluorescence ratio image of Cy5 dye moiety fluorescence intensity to Cy7 dye moiety fluorescence intensity, in which the ratio image has Cy5/Cy7 ratio value above a pre-specified threshold value; thereby identifying the location of the plurality of cancerous cells to be surgically excised from the tissue; and (v) surgically excising a region of the tissue whose perimeter encompasses the perimeter of the identified location of the plurality of cancerous cells, thereby surgically excising the plurality of cancerous cells from the subject. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

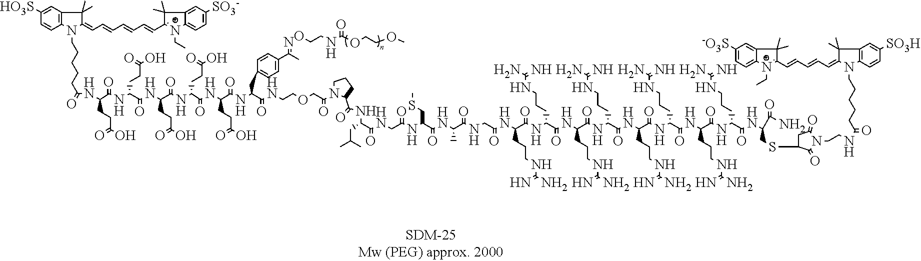

or a pharmaceutically acceptable salt thereof, for use in the preparation of an agent or a kit for surgically excising a plurality of cancerous cells from a subject in need thereof, comprising: (i) administering to the subject an effective amount of the composition, at least 12 hours prior to surgery; (ii) surgically excising a tissue from the subject to yield a surgical cavity; (iii) contacting the excised tissue with light having at least one excitation wavelength of the Cy5 dye moiety of SDM-25; (iv) capturing an image of Cy5 dye moiety fluorescence intensity in the excised tissue and, optionally, capturing an image of Cy7 dye moiety fluorescence intensity in the excised tissue; (v) detecting a region of the image of Cy5 dye moiety fluorescence intensity in which the image has a Cy5 dye moiety fluorescence intensity above a pre-specified fluorescence intensity threshold value up to 2 mm of a margin of the excised tissue; or detecting a region of the image of Cy7 dye moiety fluorescence intensity in which the image has a Cy7 dye moiety fluorescence intensity above a pre-specified fluorescence intensity threshold value up to 2 mm of a margin of the excised tissue; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value up to 2 mm of a margin of the excised tissue; thereby identifying a location of cancerous cells up to 2 mm of the margin of the excised tissue; and (vi) surgically excising from the subject additional tissue whose perimeter encompasses the identified location of the cancerous cells from the surgical cavity, thereby surgically excising the plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

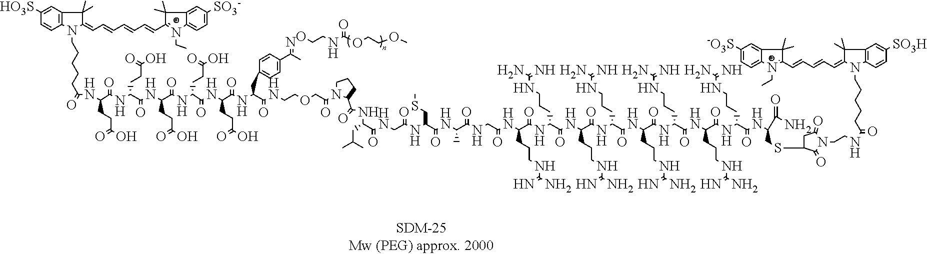

or a pharmaceutically acceptable salt thereof, for use in the preparation of an agent or a kit for evaluating a tissue suspected of having a plurality of cancerous cells, comprising: (i) administering to the subject an effective amount of the composition at least 12 hours prior to surgery; (ii) contacting the tissue with light having at least one excitation wavelength of the Cy5 dye moiety of SDM-25; (iii) capturing an image of Cy5 dye moiety fluorescence intensity in the excised tissue and, optionally, capturing an image of Cy7 dye moiety fluorescence intensity in the tissue; (iv) detecting a region of the image of Cy5 dye moiety fluorescence intensity in which the image has a Cy5 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of the image of Cy7 dye moiety fluorescence intensity in which the image has a Cy7 dye moiety fluorescence intensity above a pre-specified fluorescence intensity threshold value, thereby determining that the tissue comprises a plurality of cancerous cells; or not detecting said region and thereby determining that the tissue does not comprise a plurality of cancerous cells; or detecting a region of a fluorescence ratio image of Cy5 dye moiety fluorescence intensity to Cy7 dye moiety fluorescence intensity, in which the ratio image has a Cy5/Cy7 ratio value above a pre-specified threshold value, thereby determining that the tissue comprises a plurality of cancerous cells; or not detecting said region and thereby determining that the tissue does not comprise a plurality of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

Described herein, in certain embodiments, is a pharmaceutical composition configured in single dose form, wherein the single dose form comprises 1 mg to 16 mg of SDM-25:

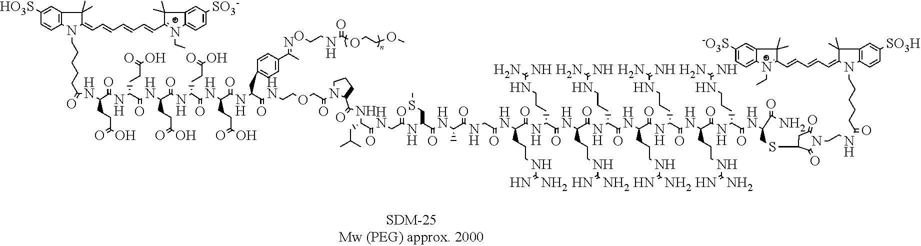

or a pharmaceutically acceptable salt thereof, for use in the preparation of an agent or a kit for imaging tissue of a subject known or suspected to contain cancerous cells either at a tissue site in vivo or excised therefrom, said use comprising: (i) intravenously or intraperitoneally administering to the subject an effective amount of the composition at least 12 hours before surgery whereby SDM-25 is taken up by cells at the tissue site; (ii) contacting tissue at said tissue site or excised therefrom with light having at least one excitation wavelength of the Cy5 dye moiety of SDM-25; (iii) capturing an image of Cy5 dye moiety fluorescence intensity in the tissue and, optionally, capturing an image of Cy7 dye moiety fluorescence intensity in the tissue; and (iv) detecting a region of the image of Cy5 dye moiety fluorescence intensity whereby the fluorescence intensity is above a determined threshold and is indicative of the presence of cancerous cells; or detecting a region of the image of Cy7 dye moiety fluorescence intensity whereby the fluorescence intensity is above a determined threshold and is indicative of the presence of cancerous cells; or detecting a region of a fluorescence ratio image of Cy5 dye moiety fluorescence intensity to Cy7 dye moiety fluorescence intensity, whereby the Cy5/Cy7 ratio value is above a determined threshold and is indicative of the presence of cancerous cells. In some embodiments, the single dose form comprises 8 mg of SDM-25 or pharmaceutically acceptable salt thereof. In some embodiments, the single dose form is formulated for intravenous administration. In some embodiments, the single dose form is formulated for intraperitoneal administration.

In some embodiments, measurements of fluorescence emission at single wavelengths are perturbed due to different tissues having different morphologies, thicknesses, cell compositions, and extracellular matrices. Ratiometric fluorescence detection utilizes a combination of fluorescent agents or indicators that generate a ratiometric change at two different wavelengths in response to a specific biological activity. In some embodiments, this methodology has been shown to reduce artifacts due to a variety of experimental factors including, for example, fluorophore concentration differences, subject motion, and variability in excitation light intensities and/or fluorescence emission collection and detection efficiencies for different fluorescence detectors and instruments.

Fluorescence Detection and Imaging Systems. Any of a variety of fluorescence detection instruments and imaging systems may be used with the imaging agents described herein, e.g., compounds comprising fluorescent dye moieties such as Cy5, Cy7, or both. For example, for in vitro studies, conventional spectrofluorometers may be used to perform cell-based assays. For imaging of tissue samples ex vivo, an epifluorescence microscope equipped with an appropriate set of excitation and emission filters and one or more CCD cameras may be utilized. For in vivo imaging applications, more sophisticated imaging systems may be employed. In specialized in vivo imaging applications, e.g., colonoscopy, a modified endoscope that allows fluorescence images to be collected at two (or more) emission wavelengths may be employed.

In general, these fluorescence detection and imaging systems will comprise: (i) one or more excitation light sources, (ii) sets of excitation and emission filters (or other components for adjusting wavelength settings and bandpass), (iii) one or more detectors, and (iv) other optical components for manipulating the path of light beams as they traverse the optical system. In some instances, the fluorescence detection and imaging systems further comprise one or more processors, computer data storage (memory) components, operating system software, instrument control software (e.g., image acquisition software), and/or data processing and display software (e.g., image processing and display software).

In some embodiments, any of a variety of light sources known to those of skill in the art are used as an excitation light source including, but not limited to, arc lamps, tungsten-halogen lamps, lasers (e.g., argon ion lasers, helium-neon (HeNe) lasers, etc.), diode lasers, light emitting diodes (LEDs), light engines, and the like, or any combination thereof. In some embodiments, the fluorescence detection or imaging system comprises at least one light source, at least two light sources, at least three light sources, at least four light sources, at least five light sources, or more.

Excitation and emission filter sets comprise any of a variety of optical filters known to those of skill in the art including, but not limited to, optical glass filters (e.g., Schott optical filters), long-pass filters, short-pass filters, interference filters, dichroic reflectors, notch filters, and the like, or any combination thereof. In some instances, the excitation and/or emission wavelengths (or bandpass) are set and/or adjusted by changing one or more optical filters in the optical path of the system. In some embodiments, the excitation and/or emission wavelengths (and bandpass) are set and/or adjusted using other components such as diffraction gratings, monochromators, acousto-optic modulators, tunable liquid-crystal filters, and the like.

In some embodiments, excitation or emission wavelength settings for the fluorescence detection or imaging system are independently adjusted and range from about 350 nm to about 900 nm. In some embodiments, the excitation or emission wavelength is set at about 350 nm, 375 nm, 400 nm, 425 nm, 450 nm, 475 nm, 500 nm, 525 nm, 550 m, 575 nm, 600 nm, 625 nm, 650 nm, 675 nm, 700 nm, 725 nm, 750 nm, 775 nm, 800 nm, 825 nm, 850 nm, 875 nm, or 900 nm. Those of skill in the art will recognize that the excitation or emission wavelengths may be set to any value within this range, e.g., about 650 nm.

In some embodiments, the bandwidths of the excitation and emission light are independently adjusted and are specified as the specified excitation or emission wavelength ±2 nm, ±5 nm, ±10 nm, ±20 nm, ±40 nm, ±80 nm, or greater. Those of skill in the art will recognize that the excitation or emission bandwidths may be set to any value within this range, e.g., about ±55 nm.

In some embodiments, a variety of detectors and image sensors known to those of skill in the art used is selected from the group consisting of photodiodes, avalanche photodiodes, photodiode arrays, photomultipliers, CCD or CMOS image sensors and cameras, and the like, and any combination thereof. In some embodiments, the fluorescence detection or imaging system comprises at least one detector, at least two detectors (e.g., for simultaneous capture of fluorescence images at two different emission wavelengths), at least three detectors, at least four detectors, at least five detectors, or more. In some optical designs, the fluorescence detection or imaging system is configured to capture fluorescence intensity images at two (or more) different emission wavelengths sequentially, e.g., by changing the fluorescence emission filter between image capture steps. In some optical designs, the fluorescence detection or imaging system is configured to collect fluorescence intensity images at two (or more) different emission wavelengths simultaneously, e.g., by including appropriate dichroic reflectors in the emission light optical path and utilizing a different detector for each emission wavelength.

Examples of other optical components that may be utilized in fluorescence detection and imaging systems include, but are not limited to, lenses or lens systems, prisms, beam-splitters, mirrors, optical fibers, diffractive optical elements for correction of chromatic aberration, etc. These components are configured, along with light sources, excitation and emission filters (or other components for adjusting wavelength settings and bandpass), and detectors, in any of a number of optical arrangements known to those of skill in the art.

Image Processing Algorithms. Also disclosed herein are image processing algorithms for use with ratiometric fluorescence imaging methods to improve the accuracy of detecting and visualizing areas of biological activity within imaged biological specimens. The disclosed algorithms for generating and displaying fluorescence ratio images and regions-of-interest (ROI) therein enable automated, semi-automated, or manual identification of ROI corresponding to the areas within the biological specimen that exhibit a specific biological activity or structure of interest. In some embodiments, ROIs are generated from fluorescence ratio images. In some embodiments ROIs are generated from a combination of fluorescence ratio images and/or fluorescence intensity images.

Computer Systems. One or more processors are used to implement the image processing methods and algorithms disclosed herein. In some embodiments, the one or more processors comprise a hardware processor such as a central processing unit (CPU), a graphic processing unit (GPU), a general-purpose processing unit, or computing platform. In some embodiments, the one or more processors comprise any of a variety of suitable integrated circuits, microprocessors, logic devices or the like. Although the disclosure is described with reference to a processor, other types of integrated circuits and logic devices, in some embodiments, are also applicable. In some embodiments, the processor has any suitable data operation capability. For example, the processor may perform 512 bit, 256 bit, 128 bit, 64 bit, 32 bit, or 16 bit data operations. The one or more processors may be single core or multi core processors, or a plurality of processors configured for parallel processing.

In some embodiments, the one or more processors, or the fluorescence imaging system itself, are part of a larger computer system and/or may be operatively coupled to a computer network (a “network”) with the aid of a communication interface to facilitate transmission of and sharing of image data and predictive results. In some embodiments, the network is a local area network, an intranet and/or extranet, an intranet and/or extranet that is in communication with the Internet, or the Internet. The network in some cases is a telecommunication and/or data network. In some embodiments, the network includes one or more computer servers, which in some cases enables distributed computing, such as cloud computing. In some embodiments, the network, with the aid of the computer system, implements a peer-to-peer network, which may enable devices coupled to the computer system to behave as a client or a server.

In some embodiments, the computer system comprises memory or memory locations (e.g., random-access memory, read-only memory, flash memory), electronic storage units (e.g., hard disks), communication interfaces (e.g., network adapters) for communicating with one or more other systems, and peripheral devices, such as cache, other memory, data storage and/or electronic display adapters. In some embodiments, the memory, storage units, interfaces and/or peripheral devices are in communication with the one or more processors, e.g., a CPU, through a communication bus, e.g., as is found on a motherboard. In some embodiments, the storage unit(s) is a data storage unit(s) (or data repositories) for storing data.

The one or more processors, e.g., a CPU, execute a sequence of machine-readable instructions, which are embodied in a program (or software). The instructions are stored in a memory location. The instructions are directed to the CPU, which subsequently program or otherwise configure the CPU to implement the methods of the present disclosure. Examples of operations performed by the CPU include fetch, decode, execute, and write back. The CPU may be part of a circuit, such as an integrated circuit. One or more other components of the system may be included in the circuit. In some embodiments, the circuit is an application specific integrated circuit (ASIC).

The storage unit stores files, such as drivers, libraries and saved programs. The storage unit stores user data, e.g., user-specified preferences and user-specified programs. The computer system in some cases, in some embodiments, includes one or more additional data storage units that are external to the computer system, such as located on a remote server that is in communication with the computer system through an intranet or the Internet.

Some embodiments of the image processing methods provided herein, such as the disclosed image processing algorithms, are implemented by way of machine (e.g., processor) executable code stored in an electronic storage location of the computer system, such as, for example, in the memory or electronic storage unit. The machine executable or machine readable code is provided in the form of software. During use, the code is executed by the one or more processors. In some embodiments, the code is retrieved from the storage unit and stored in the memory for ready access by the one or more processors. In some situations, the electronic storage unit is precluded, and machine-executable instructions are stored in memory. The code may be pre-compiled and configured for use with a machine having one or more processors adapted to execute the code, or may be compiled at run time. The code, in some embodiments, is supplied in a programming language that is selected to enable the code to execute in a pre-compiled or as-compiled fashion.

In some embodiments, various aspects of the technology are “products” or “articles of manufacture” typically in the form of machine (or processor) executable code and/or associated data that is stored in a type of machine readable medium. In some embodiments, machine-executable code is stored in an electronic storage unit, such as memory (e.g., read-only memory, random-access memory, flash memory) or on a hard disk. “Storage” type media include any or all of the tangible memory of the computers, processors or the like, or associated modules thereof, such as various semiconductor memory chips, tape drives, disk drives and the like, which, in certain embodiments, provide non-transitory storage at any time for the software that encodes the image processing methods and algorithms disclosed herein.

In some embodiments, all or a portion of the software code is communicated through the Internet or various other telecommunication networks. Such communications, for example, enable loading of the software from one computer or processor into another, for example, from a management server or host computer into the computer platform of an application server. Thus, other types of media that are used to convey the software encoded instructions include optical, electrical and electromagnetic waves, such as those used across physical interfaces between local devices, through wired and optical landline networks, and over various atmospheric links. The physical elements that carry such waves, such as wired or wireless links, optical links or the like, are also considered media that convey the software encoded instructions for performing the methods disclosed herein. As used herein, unless restricted to non-transitory, tangible “storage” media, terms such as computer or machine “readable medium” refer to any medium that participates in providing instructions to a processor for execution.

In some embodiments, the computer system includes, or is in communication with, an electronic display for providing, for example, images captured by the imaging device. The display is typically also capable of providing a user interface. Examples of UI's include, but are not limited to, graphical user interfaces (GUIs), web-based user interfaces, and the like.

### EXAMPLES

Abbreviations: CAN: Close As Negative; SDS: Same Day Surgery; DBS: Day Before Surgery; PM: Positive Margin; FP: False Positive.

### Example 1. Preparation of SDM-25 Formulation

An exemplary synthesis of SDM-25 is as follows. Those skilled in the art will recognize that starting materials and reaction conditions may be varied to produce the desired end products.

### Materials and Methods

HPLC-grade acetonitrile was purchased from Fisher Scientific (Phillipsburg, PA). Purified water was collected through Milli-Q water purification system (Millipore, Bedford, Mass.). 3-Maleimidopropionic acid-Pfp ester was purchased from Molecular Biosciences (Boulder, Colo.). PBS-EDTA buffer was purchased from Teknova (Hollister, Calif.). Trifluoroacetic acid (TFA), Dimethylformamide (DMF) and N-methylmorpholine (NMM) were supplied by Sigma-Aldrich (Milwaukee, Wis.). α-Mercaptoethyl-ω-methoxy, poly-oxyethylene (Mw 2,000, 5,000, 20,000 and 40,000) [mPEG(2K)—SH, mPEG(5K)—SH, mPEG(20K)—SH, mPEG(40K)—SH] and α-aminoxyl-w-methoxy, polyoxyethylene (Mw 2,000, 5,000, 20,000 and 40,000) [mPEG(2K)—ONH₂, mPEG(5K)—ONH₂, mPEG(20K)—ONH₂, mPEG(40K)—ONH₂] were purchased from NOF America Corporation (Irvine, Calif.). mPEG(1K)—NHNH₂ was purchased from Nanocs (New York). TRDye 800CW maleimide (Mal-IRDye) and TRDye 750 succinimidyl ester were supplied by Li-Cor Biosciences (Lincoln, Nebr.). Lyophilized peptide P-3 was prepared using standard resin based peptide coupling methods:

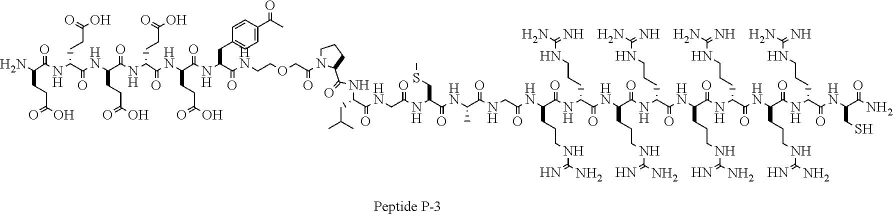 [SMILES: CSC[C@H](NC(=O)CNC(=O)[C@H](CC(C)C)NC(=O)[C@@H]1CCCN1C(=O)COCCNC(=O)[C@H](Cc1ccc(C(C)=O)cc1)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@H](N)CCC(=O)O)C(=O)N[C@@H](C)C(=O)NCC(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CCCNC(=N)N)C(=O)N[C@H](CS)C(N)=O]

LC-MS analysis was carried out on an Agilent 1200 SL series in combination with AB SCIEX API 3200, equipped with CTC PAL autosampler operating at 4° C., a vacuum degasser, binary pump, UV-VIS detector, associated Analyst 1.5 analytical software and a Phenomenex column (Kinetex 2.6μ C18 100A, 100×2.1 mm) or a Waters 2695 separation module equipped with a Waters 2487 dual λ absorbance detector in combination with Finnigan LCQ Deca XP mass spectrometer. The equipment is associated with Xcalibur analytical software and a Peeke Scientific column (Titan 200 5 μm, C18-MC, 50×2.1 mm).

Preparation HPLC were carried out on an Agilent system (Agilent 1200 series) and a Thermo Scientific column (Hypersil Gold C18, 5μ, 250.times.10 mm), or a Waters Delta Prep preparative HPLC System and a Varian column (F75L, C18, 15μ, 1200 g), or a Waters PrepLC System equipped with a Waters 2487 dual .lamda. absorbance detector, Fraction Collector III, Masslynx software and a Thermo Scientific column (Hypersil Gold C18, 5.mu., 250×10 mm) or a Phenomenex column (luna, C18(2), 5μ, 100A AX 150×30 mm). The mobile phase consisted of a water (0.05% TFA)(solvent A)/acetonitrile (0.05% TFA)(solvent B) gradient.

Centrifugation was carried out at 4° C. with an Eppendorf centrifuge 5810R or a Beckman Microfuge® 18.

### Synthesis of Intermediate 1

To a solution of peptide P-3 (200 mg, 49.6 μmol) in DMF (5 mL) at room temperature in the dark were added Cy5 maleimide (60 mg, 65.6 μmol) and N-methylmorpholine (80 μL, 0.73 mmol) with stirring. The reaction was followed by LC-MS and completed in 1 h. Ether (40 mL) was added to the mixture. The precipitate was collected after centrifuge, washed with ether (40 mL×2) and purified by HPLC to afford intermediate 1 (141 mg, 61%). Calculated: [M+3H]³⁺ (C₁₅₂H₂₄₂N₅₁O₄₃S₄) m/z=1200. Found ESI: [M+3H]³⁺ (C₁₅₂H₂₄₂N₅₁O₄₃S₄) m/z=1200.

### Synthesis of Intermediate 2

To a solution of intermediate 1 (101 mg, 21.8 μmol) in DMF (10 mL) at room temperature were added Cy7 carboxylic acid, succinimidyl ester (40 mg, 41.1 μmol) and N-methylmorpholine (0.2 mL, 1.8 mmol). The resulting mixture was stirred at room temperature in the dark for 36 h. Ether (35 mL) was added to the mixture. The precipitate was collected after centrifuge and washed with ether (40 mL×2). Purification of the mixture by RP-HPLC afforded intermediate 2 (28.1 mg, 25%) and intermediate 1 (63 mg). Calculated: [M+3H]³⁺ (C₁₈₇H₂₈₂N₅₃O₅₀S₆) m/z=1421. Found ESI: [M+3H]³⁺ (C₁₈₇H₂₈₂N₅₃O₅₀S₆) m/z=1421.

### Synthesis of SDM-25

The mixture of intermediate 2 (28.1 mg, 5.4 μmol) and mPEG(2K)—ONH₂ (17 mg, 7.6 μmol) in glycine buffer (4 mL, 0.1 M, 20 mM aniline, pH 3.0) and acetonitrile (0.8 mL) was stirred at room temperature in the dark for 24 h. After the reaction was complete, acetophenone (10 μL, 86 μmol) was added. The mixture was stirred at room temperature for 2 h. Purification by RP-HPLC afforded SDM-25 (25 mg, 63%).

In the studies described in Examples 2 and 3 below, SDM-25 is provided in a Sterile Solution and was administered once on Day 1 via intravenous (IV) infusion.

### Example 2. A Phase 1, Open-Label, Dose-Escalation Study of SDM-25 in Women Undergoing Surgery for Primary, Nonrecurrent Breast Cancer

This study was a Phase 1 open-label, dose-escalation study that evaluated 5 dose levels (1, 2, 4, 8, and 16 mg) of SDM-25 in women undergoing surgery for primary, nonrecurrent breast cancer. SDM-25 was administered as an IV infusion over 30 minutes.

TABLE 1
SDM-25 Dose Levels and Number of Subjects Treated

| | | | | Number of Subjects |
|---|---|---|---|---|
| | | | | Enrolled and |
| Study Period | Cohort | Dose | | Treated |
| Dose Escalation | 1 | 1 | mg | 3 |
| | 2 | 2 | mg | 3 |
| | 3 | 4 | mg | 3 |
| | 4 | 8 | mg | 3 |
| | 6 | 16 | mg | 3 |
| Dose Expansion | 5 | 8 | mg | 12 |

The primary objectives of the study were to (1) evaluate the safety and tolerability of SDM-25 administered by slow IV infusion to women with primary, nonrecurrent breast cancer undergoing surgery; and (2) characterize the PK of SDM-25 in women with primary, nonrecurrent breast cancer.

Secondary objectives were to (1) determine the dose of SDM-25 needed to generate a fluorescence signal in tumor and lymph-node tissue that was sufficiently high to enable fluorescence recordings and image analysis with a camera system; and (2) evaluate the effect of timing of administration (day of surgery versus day before surgery) of SDM-25 administration relative to surgery on optical fluorescence characteristics.

A total of 27 subjects were enrolled and treated in the study. Subjects had ductal carcinoma in situ or Stage I to III primary invasive carcinoma of the breast. Before being enrolled in the study, the subject's primary surgical treatment was already planned to be a mastectomy or lumpectomy, with SLNB or ALND (according to the method preferred by the surgeon [Investigator]) as part of the subject's planned therapy.

The study included 5 cohorts (Cohorts 1 to 4 and Cohort 6) using a 3+3 cohort expansion design followed by an expansion cohort (Cohort 5) at the maximum-tolerated dose (MTD) or a dose determined by the Safety Review Committee (SRC) to compare fluorescence characteristics at different SDM-25 administration times before surgery. There were no dose-limiting toxicities (DLT) in the study and an MTD was not reached. On the basis of the PK, imaging analysis, and safety data, the dose of 8 mg was selected for the dose-expansion period of the study.

Subjects in the dose-escalation period of the study (Cohorts 1 to 4 and Cohort 6; 15 subjects) were administered SDM-25 at 12 to 20 hours before surgery (ie, the day before surgery). Subjects in the dose-expansion period of the study (Cohort 5; 12 subjects) were administered SDM-25 either (1) 2 to 12 hours in advance of surgery (the day of surgery) (8 subjects), or (2) 12 to 20 hours in advance of surgery (the day before surgery) (4 subjects). Subjects were followed until 30 days (±3 days) after administration of SDM-25, with follow-up clinic visits at 7 days (±2 days), 14 days (±3 days), and 30 days (±3 days) after administration of SDM-25.

Fluorescence imaging was performed with the fluorescence camera system attached to a movable arm to enable the camera (Artemis Imaging System, Model Artemis-AVB) to be positioned over the surgical field, thereby allowing the surgeon to see the fluorescence image displayed on a color monitor superimposed onto, or separate from, the surgical field. Because the protocol stipulated that imaging was not to be viewed by the surgeon during the course of surgery, the surgeon did not make any changes in her operating procedure based on imaging. The freshly resected tissue, including both the primary tumor-containing specimen and any secondary breast specimens and/or lymph nodes, underwent fluorescence intensity and ratio imaging in the operating room before being sent for histopathologic analysis. Results from fluorescence imaging data were correlated with results from the histopathologic analysis.

### Exemplary Results of Phase 1 Study

SDM-25 was shown to be safe and generally well-tolerated in this study. There were no deaths or DLTs during the study, and no subjects discontinued treatment with SDM-25 due to a treatment-emergent adverse event (TEAE). The study included a 5-cohort dose-escalation period (1, 2, 4, 8, and 16 mg), followed by an expansion cohort to compare fluorescence characteristics at different SDM-25 administration times before surgery. In the dose-escalation period, although a dose of 16 mg SDM-25 resulted in a brighter signal, 8 mg SDM-25 appeared to give better separation between positive and negative tissue for both fluorescence ratio and intensity to distinguish tumor from adjacent tissue. The tumor ratio was 66% higher than the adjacent tissue ratio for subjects in the dose-escalation period who received with 8 mg SDM-25 (Cohort 4) compared with only 48% higher for subjects in the dose-escalation period who received 16 mg SDM-25 (Cohort 6). Because of the better contrast seen with the 8 mg dose in Cohort 4, 8 mg was chosen for the dose-expansion cohort. The dose-expansion period of the study evaluated the time dependence of fluorescence imaging. Specifically, imaging properties and fluorescence response on the same day (SDS group—2 to 12 hours before surgery) and the day after (DBS group—12 to 20 hours before surgery) SDM-25 dosing were explored.

In the dose-expansion period of the study, imaging performed on the same day of surgery ranged from approximately 2.5 to 6.5 hours after SDM-25 dosing. A total of 6 subjects were evaluated under these conditions. In the 6 subject samples analyzed, Cy5 fluorescence intensities and Cy5/Cy7 fluorescence ratios were higher in primary tumors compared with putative nonmalignant adjacent tissue. Positive lymph nodes in many cases could be identified.

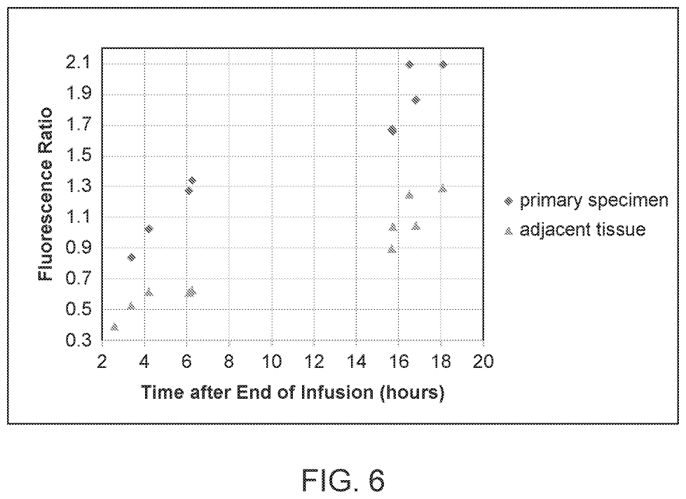

In the dose-expansion period of the study, imaging performed on the day after dosing (DBS group) for 4 subjects (Cohort 5) yielded similar results to those achieved for the 3 subjects in the dose-escalation period of the study who also received 8 mg SDM-25 (Cohort 4) the day before surgery/imaging (DBS group). Imaging conditions were similar for the 2 dosing groups, with imaging being performed approximately 15 to 18 hours after SDM-25 dosing (DBS) and between 2 and 8 hours for same day dosing (SDS). For these 7 DBS and 6 SDS patients, the primary tumor had higher fluorescence intensities and Cy5/Cy7 fluorescence ratios than putatively nonmalignant adjacent tissue. For example, FIG. 6 depicts larger differences in the fluorescence ratios of Cy5 to Cy7 (Cy5/Cy7) between primary tumor specimens removed and adjacent nonmalignant tissue at times greater than 2 hours after the end of SDM-25 infusion and at time points between 14 and 20 hours after the end of SDM-25 infusion, indicating that same-day dosing (SDS) and day before dosing (DBS) yielded similar imaging benefits. These data indicated that separation of primary tumor and putatively nonmalignant adjacent tissue can be achieved using both same day and day before dosing. It also indicated that both SDS and DBS dosing had similar promise.

### Example 3. An Open-Label, Single-Arm, Phase 2 Study of SDM-25 in Women with Primary, Nonrecurrent Breast Cancer Undergoing Surgery

This was a Phase 2, open-label study in women with primary, nonrecurrent, and nonmetastatic breast cancer undergoing surgery. The primary objective was to evaluate the effectiveness of the SDM-25 to detect positive or close margins and/or residual malignant cancer following resection of tissue (Period 2). The study evaluated approximately 120 patients who received SDM-25 and underwent imaging. Eligible patients were enrolled into Period 1 or Period 2 of the study. In Periods 1 and 2, patients received a single IV dose of 8 mg SDM-25 over 30 minutes. After the completion of the dose, surgeons waited to bring the patient to the surgical suite anywhere from 2-24 hours (Period 1) or 3-20 hours (Period 2).

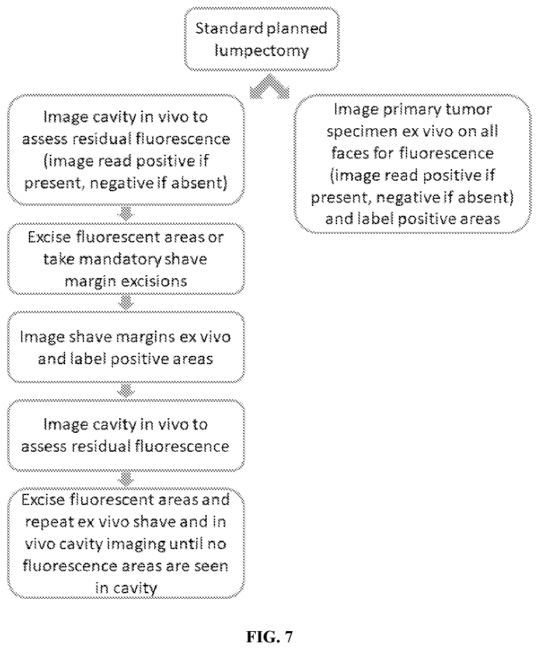

There was an initial surgical removal of breast cancer tissue for patients treated in Period 1 and Period 2 that was not guided by fluorescence images generated by SDM-25 administration. At the conclusion of the initial resection, the camera was utilized to identify fluorescence signals above the set threshold in the cavity and on the surface of removed tissue. In Period 1 the Investigator used his/her clinical judgment in performing additional resections with knowledge of the imaging data. In Period 2, Investigators were required to remove additional tissue from the cavity based on fluorescence imaging after the initial removal of tissue. All additional removed tissue was imaged with the camera system as well as the residual cavity. Additional tissue was removed until there was no more fluorescence above threshold seen in the cavity or the surgeon was satisfied that no more tissue needed to be removed. (see FIG. 7 for schematic of the procedure). The imaging results were then compared with the pathology results for that specific area of tissue. Areas with fluorescence above the threshold were predicted to have malignant tissue at or close to the cut surface of that tissue.

### Period 1

The purpose of Period 1 was to evaluate the exploratory display thresholds, based on fluorescence Cy5/Cy7 ratio and intensities, and make adjustments to achieve maximal differences in the fluorescent signals between malignant and nonmalignant tissue. SDM-25 was well tolerated in 32 women administered a single IV dose of 8 mg SDM-25 over 30 minutes in Period 1. In Period 1: 82% of patients with intraoperative positive margins identified using best threshold and similar analysis post hoc.

### Period 2

The purpose of Period 2 was to test the dosing and imaging conditions determined in Period 1 and the accuracy of the SDM-25 imaging data to distinguish between malignant and nonmalignant tissues in a larger group of patients. The primary objective was to evaluate the effectiveness of the SDM-25 to detect positive or close margins and/or residual malignant cancer following resection of tissue. Period 2 was initiated to test pre-specified fluorescence intensity thresholds derived from Period 1 data and for surgeons to perform fluorescence directed shaves of margins. Variables that were considered included, but were not limited to: time between SDM-25 administration and imaging, stage of disease, corresponding pathology findings and prior therapy.

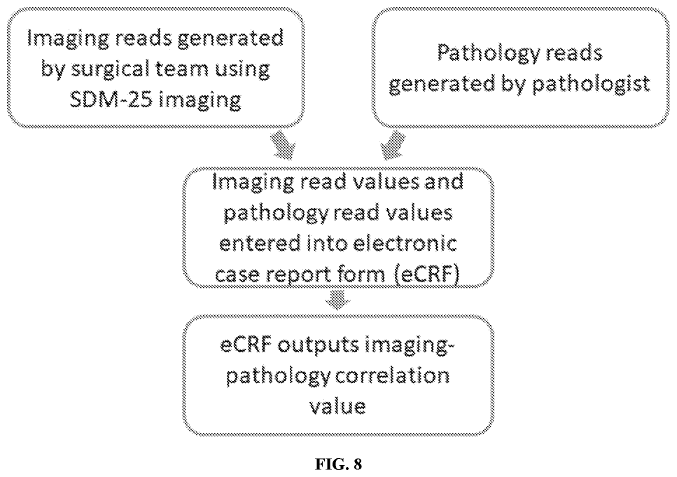

Fluorescence intensity and/or ratio imaging results (in vivo data points) included: image of the initial surgical field before tumor excision; image of the initial tumor cavity after excision; image of the final tumor cavity (after final resection); initial axillary field image; sentinel node images; and final axillary field. Fluorescence intensity and/or ratio imaging results (ex vivo data points) included: surfaces of the primary tumor specimen (superior, medial, inferior, lateral, posterior and anterior faces); transected primary tumor specimen; shave margins of primary tumor specimen; surfaces of any secondary specimens including additional margins; and images of all resected nodes. At the sample level, pictures of each area of tissue were assessed by the surgical team as to whether there was tissue that was above the fluorescent threshold. This was compared against the pathologist determination of the pathologic status of the tissue in that same region as to whether the tissue had invasive cancer tissue or non-invasive cancer tissue (e.g. DCIS) at or close to the surface. (FIG. 8). A determination was then made for each sample as to whether the sample was a true positive, true negative, false positive, false negative, or indeterminate (Table 2).

TABLE 2
Imaging-Pathology Correlation Value

Imaging Read Value

| | | Negative | Positive |
|---|---|---|---|
| Pathology | Negative | True Negative | False Positive |
| Read | Positive | False Negative | True Positive |
| Value | Unknown | Indeterminate | Indeterminate |

At the patient level, a patient classified as a true positive required at least one correctly identified positive margin sample. A false negative patient required at least one false negative sample if there were no true positive samples. A true negative patient required all negative margin samples to be correctly identified as negative by fluorescence. By a second analysis, a definition of the patient level result which allowed a clinically relevant true negative patient to have up to one false positive sample per patient was also evaluated. And thus, a clinically relevant false positive patient required more than one false positive sample per patient while having no true positives or false negative samples. Determination of the pathologic status of each margin sample was as follows. Invasive cancer was divided into 3 groups: cancer at the surface of the lumpectomy/mastectomy specimen (positive), cancer not touching the surface (0 mm away) but within 2 mm of the surface (close) and cancer more than 2 mm away (negative). Ductal Carcinoma in Situ (DCIS) was divided into 2 groups, within 2 mm (positive) and greater than 2 mm (negative). Pathologic status of a margin was then determined by assessing whether any given margin sample was positive for either DCIS or invasive cancer. If either was true, this was treated as a positive margin. For the purposes of analysis, 3 different approaches were used: treating “close” invasive margins as positive (CAP), treating “close” invasive margins as negative (CAN), and consensus definition.

### Period 2 Results

92 patients were dosed with SDM-25, of which 87 were evaluable for efficacy.

Tumors were characterized as follows: 81% ER+ and 79% PR+; 8% HER2+; 8% triple negative; tumor types at 62% invasive, 27% mixed invasive/DCIS; 11% DCIS only; invasive types at 76% ductal; 13% lobular; 6% mucinous; and 6% other.

Demographics of the patients were as follows: Age: Average 59.5 years and Median 61 years; Race: 79% White, 12% African American, 2% Asian, 1% Native American, 7% Not Reported; Ethnicity: 87% Non-Hispanic, 11% Hispanic, 2% Not Reported/Unknown.

The primary endpoint of the study, fluorescence detection of patients with residual cancer, was met with high statistical significance for both dosing groups and both margin definitions. The primary endpoint in the study was the proportion of total patients that had correctly identified residual cancer, also known as a positive margin, when on outer surface of excised specimens was evaluated. In the CAN case, 27.7% of DBS patients had positive margin detected by fluorescence (p<0.001) and 12.5% of SDS patients had positive margin detected (p=0.048).

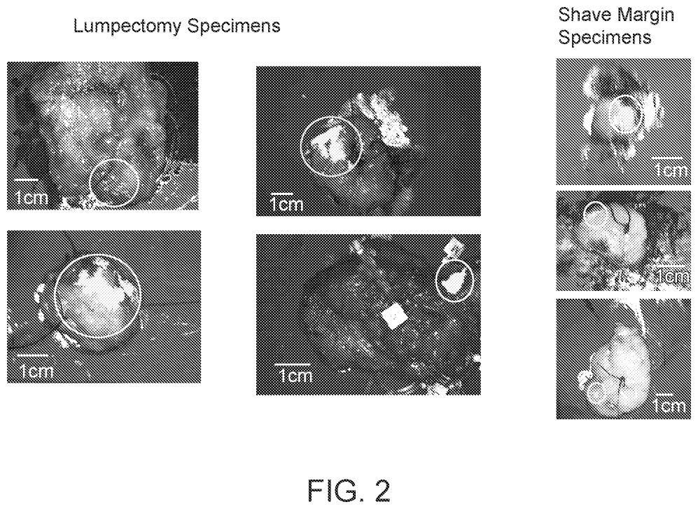

For secondary endpoints, key results included: SDM-25 imaging correctly detected and visualized pathology confirmed positive and close margins (lumpectomy/mastectomy surfaces and cavity shaves, see FIG. 2 for examples of correctly identified positive margins visualized as a lightly-shaded overlay superimposed on white light image of excised primary specimen and cavity shaves) in up to 75% the patients; patient level specificity provides the proportion of negative margins patients identified and values ranged from approximately 60% to 87%, using the clinically-relevant definitions, with DBS dosing generally being higher than SDS values; patient level false positives are sufficiently low as to not cause substantial removal of non-malignant breast tissue and prolongation of surgery; and data from sample level sensitivity indicate that detection with day before dosing (DBS) of SDM-25 is superior for detecting positive margins than same day of surgery (SDS) dosing.

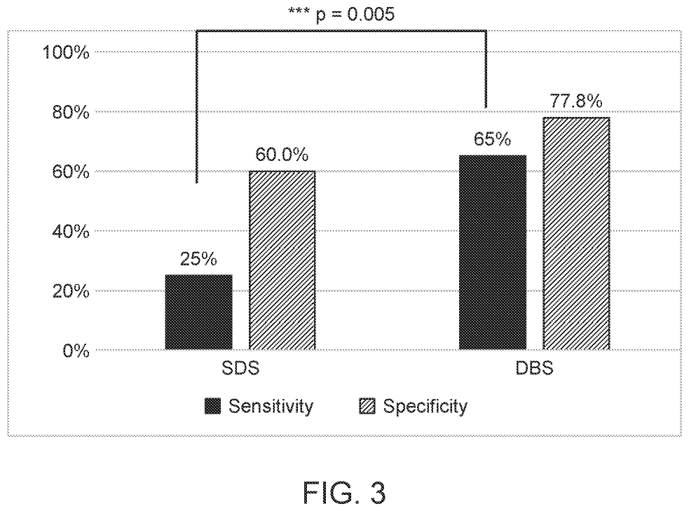

Patient level sensitivity in margin-relevant tissue. In the CAN case, the DBS group had a sensitivity of 65.0%) (FIG. 3). Of the 7 patients missed, 2 of them had a correctly identified close margin. Therefore, 75.0% of the PM CAN patients had a positive or close margin detected. The SDS group sensitivity was 25.0% and the difference between dosing groups was significant (p=0.005), indicating that DBS dosing is more sensitive in detecting cancer with pre-specified thresholds.

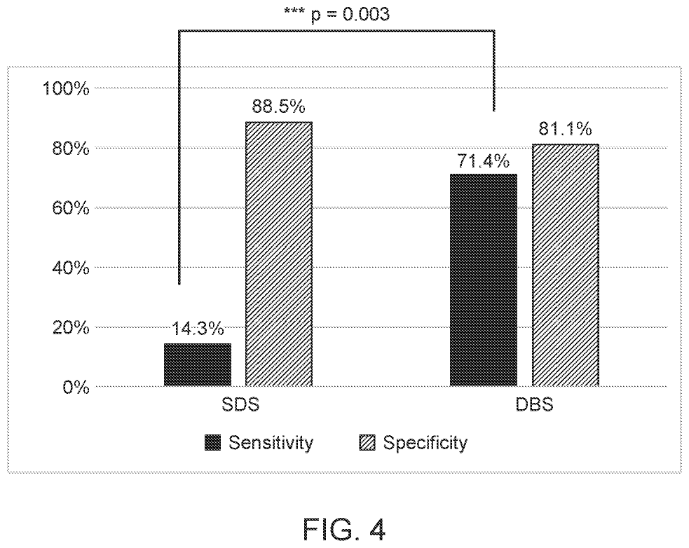

Improved SDM-25 DBS cancer detection compared to SDS dosing was found in both lumpectomy/mastectomy and shave specimens using relevant CAN PM definition. For surgical cavity shaves the sensitivity was 71.4% for DBS dosing compared to 14.3% for SDS dosing (FIG. 4). This large difference was unexpected, given earlier results from the Phase 1 study, and highly significant (p=0.003). Furthermore, for shaves the specificity was 88.5% for SDS dosing and 81.1% for DBS dosing (FIG. 4). Without wishing to be bound by theory, a hypothesis is that more time may be needed to achieve sufficient SDM-25 proteolytic cleavage to generate sufficient fluorescence change due to the relatively small tumor and sample size in shave specimens and being furthest from primary tumor (perhaps lower absolute enzyme concentrations in these specimens). Since the shaves are small and originate from the surgical cavity bed, these specimens are crucial for determination of final margin status. For the lumpectomy/mastectomy surfaces, the sensitivity values were 47.4% for DBS compared to 26.7% for SDS using same CAN definition. The DBS sensitivity was 77.5% higher than that of SDS for lumpectomy/mastectomy surfaces.

Patient level specificity provides the proportion of negative margins patients identified and values ranged from approximately 60% to 87%, using clinically relevant definitions, with DBS dosing generally being higher than SDS values. The patient level specificity for CAN case was 77.8% for DBS group and 60.0% for the SDS (FIG. 3).

The proportions of patients with a false positive and clinically relevant false positives were tabulated and broken out by SDS and DBS dosing. In the case of clinically relevant FP the values drop to 10% and 4.3%. In the CAN case, the clinically relevant FP were 20.0% and 12.8% for SDS and DBS patients, respectively. The DBS group surprisingly had lower false positive patients that SDS group.

The overall sample fluorescence detection accuracy (i.e., the percentage of samples assigned a correct prediction of True Positive or True Negative) of margin-relevant tissues (lumpectomy/mastectomy and cavity shaves) for DBS dosing was 79.5% (435/547) in the CAN case. The sample specificity was similar for both dosing groups and PM definitions and ranged from 84-87%. The sample false positive rate (FPR) is equal to 1-specificity. Therefore, the sample FPRs were also similar across groups ranging from 13-16%. The lowest FPR was 7.4% in the SDS group in cavity shaves. This result is likely due to inferior cancer detection compared to DBS group.

Sample level sensitivity supports that DBS detection is superior to SDS for detecting PMs. In pathology positive samples, there was a larger and significant difference in true positive rates between the dosing groups for margin-relevant tissue (lumpectomy/mastectomy specimen surfaces and cavity shaves). In CAN case, the true positive sample rate for DBS was 39.2% compared to only 12.0% for SDS dosing. This 3.3× greater detection rate for DBS was highly significant (p<0.001). Breaking the result down further between lumpectomy and shaves, the superior detection rate was significantly observed for DBS group for both tissue types. In lumpectomy/mastectomy tissue, the true positive rate was 40.5% compared to only 15.8% for SDS group, (p=0.013). And in shave tissue the rate was 35.7% compared to 8.1%, (p=0.042).

### Patient Clinical Benefit

Based on the clinical imaging results, SDM-25 can benefit the majority of patients undergoing curative lumpectomy that would have had an interoperative PM, which could have resulted in re-excision recommendation. Patients who benefit the most from SDM-25 with imaging include those that have a PM identified intraoperatively. These patients have improved potential for removal of all cancer, achieving a clean margin, and preventing a re-excision operation. For those patients with correctly identified clinically relevant negative margins, the imaging provides additional intraoperative benefit confirming that the surgical procedure has been successful allowing the surgeon confidence to stop removing additional tissue. The highest patient level sensitivity was observed in the DBS group, compared to SDS group, and using the CAN definition, in this case, 27.7% of patients have a correctly identified PM and 44.6% have correctly identified clinically relevant negative margins. Together, 72.3% of patients have benefit.

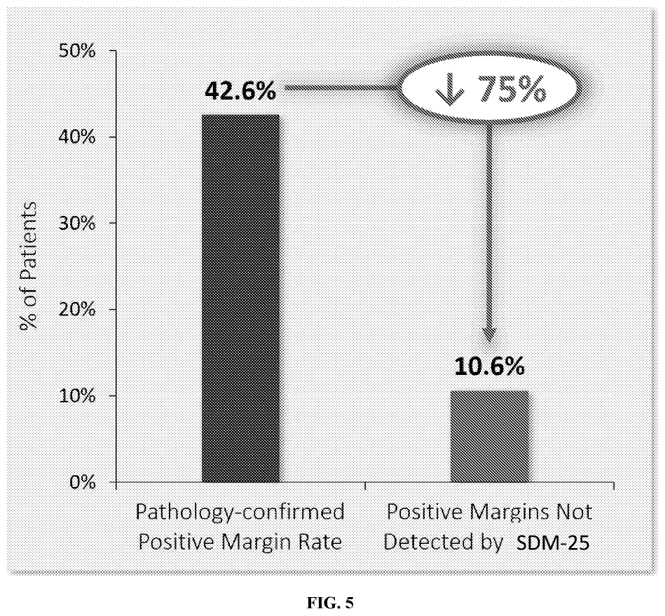

Further, FIG. 5 depicts that the SDM-25 imaging method described intraoperatively identified up to 75% patients with a cancer within 2 mm of surface (close margin) in margin-relevant tissues (lumpectomy surfaces and cavity shaves) in patients dosed DBS. The intraoperative positive margin rate was 42.6% in patients dosed DBS as determined by pathology. This result indicates that up to 75% of patients that could otherwise be candidates for a re-excision operation may be helped and avoid a second operation.

### Example 4: Conversion of SDM-25-Identified Positive Margins to Final Negative Margins

A clinically important objective in breast cancer surgery is to reduce the number of patients who after surgery have positive margins. To determine how SDM-25 cancer visualization may aid in the reduction of positive margins, an analysis was conducted to evaluate the number of patients who had intraoperative PMs identified by SDM-25 during the study described in Example 3 that were then converted to negative margins during the same operation. For this analysis PMs were determined using the same methodology as used in the sensitivity analysis. For the final negative margin (FNM), this was abstracted from the synoptic summary of the final margin status in the final pathology report for the patient. When the synoptic report did not specifically indicate the final margin status for the case, then this was derived by utilizing the final status of the shave margins and lumpectomy specimens utilizing the criteria from the Society of Surgical Oncology/American Society of Clinical Oncology consensus guidelines.

Results for this analysis are shown in Table 3. Conversion to NMs was reported in 60.0% and 76.9% of the SDS and DBS patients initially identified with PMs, respectively. The proportion of patients identified with PMs who were then converted to final NMs to the total evaluable population was higher for the DBS patients (21.3%) as compared to the SDS patients (7.5%). This difference is due to the higher patient-level sensitivity observed in the DBS group. These results indicate that SDM-25 fluorescence imaging guidance resulted in more conversions of PMs to FNMs in the DBS patient group relative to SDS patient group and that DBS administration of SDM-25 is the preferred clinical approach.

TABLE 3
Patient-Level Identified Positive Margins^{a} Converted
to Final Negative Margins With SDM-25

| | Statistic | SDS | DBS |
|---|---|---|---|
| Evaluable Patients | n | 40 | 47 |
| Intraoperative Positive Margin Patients | n | 20 | 20 |
| Identified by Postoperative | | | |
| Histopathology | | | |
| Intraoperative Positive Margin Patients | n | 5 | 13 |
| Correctly Identified by SDM-25 | | | |
| SDM-25 Identified Positive Margin | n | 3 | 10 |
| Patients Converted to FNMs^{b} | | | |
| Conversion Rate of SDM-25 Identified | % | 60.0% | 76.9% |
| Positive Margin Patients to FNMs | (n) | (3/5) | (10/13) |
| Proportion of SDM-25 Identified Positive | % | 7.5% | 21.3% |
| Margin Patients Converted to FNMs to | (n) | (3/40) | (10/47) |
| Total Evaluable Patients | | | |

Abbreviations:
CAN = close as negative;
DBS = day before surgery dosing;
FNM = final negative margin;
SDS = same day as surgery dosing
^{a}Margins determined utilizing imaging agent SDM-25 (using CAN positive margin definition) were compared to results determined by histopathology.
^{b}Converted patients identified by Avelas from final pathology reports.

### Example 5: Positive Margin Detection with Consensus Definition

In addition to CAN and CAP definition of positive margin, an analysis of clinical data from the study described in Example 3 was done using the consensus pathology definition where patients with a mix of invasive and non-invasive (e.g. DCIS) cancers are treated the same as invasive cancer (Moran et al 2014, Morrow et al 2016). Consensus pathology was determined from pathology records and pathology reports.

Using the consensus definition, the DBS patient level sensitivity was 77% [10/13] with consensus pathology which was even higher than the DBS patient level sensitivity of 65% [13.20] for CAN pathology (see Table 4). This result further supports that patient-level sensitivity is high using multiple PM definitions and including one used by majority of pathologists.

TABLE 4
Day Before Surgery Group, Patient-Level Results

| Positive Margin | | |
|---|---|---|
| Definition Method | CAN | Consensus |
| TN | 21 | 24 |
| TP | 13 | 10 |
| FN | 7 | 3 |
| FP | 6 | 10 |
| Total | 47 | 47 |
| Sensitivity | 65% | 77% |
| Specificity | 78% | 71% |

Abbreviations:
CAN = close as negative;
FN = false negative;
FP = false positive;
TN = true negative;
TP = true positive

1. (canceled)

2. A method of surgically excising a plurality of cancerous cells from a subject in need thereof, comprising:
(i) administering to the subject an effective amount of SDM-25, 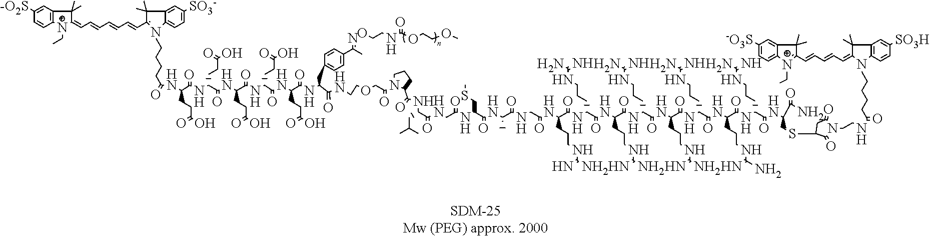
or a pharmaceutically acceptable salt thereof, at least 12 hours prior to surgery;
(ii) during surgery, contacting a tissue suspected to comprise a plurality of cancerous cells with light having at least one excitation wavelength of the Cy5 dye moiety of SDM-25;
(iii) capturing an image of Cy5 dye moiety fluorescence intensity in the tissue and, optionally, capturing an image of Cy7 dye moiety fluorescence intensity in the tissue;
(iv) detecting a region of the image of Cy5 dye moiety fluorescence intensity in which the image has a Cy5 dye moiety fluorescence intensity above a pre-specified fluorescence intensity threshold value; or
detecting a region of the image of Cy7 dye moiety fluorescence intensity in which the image has a Cy7 dye moiety fluorescence intensity above a pre-specified fluorescence intensity threshold value; or
detecting a region of a fluorescence ratio image of Cy5 dye moiety fluorescence intensity to Cy7 dye moiety fluorescence intensity, in which the ratio image has a Cy5/Cy7 ratio value above a pre-specified threshold value;
thereby identifying the location of the plurality of cancerous cells to be surgically excised from the tissue; and
(v) surgically excising a region of the tissue whose perimeter encompasses the perimeter of the identified location of the plurality of cancerous cells, thereby surgically excising the plurality of cancerous cells from the subject.

3. (canceled)

4. The method of claim 2, wherein SDM-25, or pharmaceutically acceptable salt thereof, is administered to the subject at least 14, 15, 16, 17, 18, 19, 20, or 21 hours prior to surgery.

5. The method of claim 2, wherein SDM-25, or pharmaceutically acceptable salt thereof, is administered to the subject at least 14 hours prior to surgery.

6. The method of claim 2, wherein SDM-25, or pharmaceutically acceptable salt thereof, is administered to the subject at least 15 hours prior to surgery.

7. The method of claim 2, wherein SDM-25, or pharmaceutically acceptable salt thereof, is administered to the subject at least 16 hours prior to surgery.

8. The method of claim 2, wherein SDM-25, or pharmaceutically acceptable salt thereof, is administered to the subject at least 17 hours prior to surgery.

9. The method of claim 2, wherein SDM-25, or pharmaceutically acceptable salt thereof, is administered to the subject at least 18 hours prior to surgery.

10. The method of claim 2, wherein SDM-25, or pharmaceutically acceptable salt thereof, is not administered to the subject more than 21 hours before surgery, preferably, not more than 20.5 hours before surgery, more preferably, not more than 20 hours before surgery.

11. The method of claim 2, wherein SDM-25, or pharmaceutically acceptable salt thereof, is intravenously administered to the subject.

12. The method of claim 2, wherein the at least one excitation wavelength is about 650 nm.

13. The method of claim 2, wherein the plurality of cancerous cells is of a cancer selected from the group consisting of AIDS-related cancers, anal cancer, basal cell carcinoma, bile duct cancer, bladder cancer, bone cancer, breast cancer, cervical cancer, colon cancer, colorectal cancer, endometrial cancer, ependymoma, esophageal cancer, eye cancer, gastric (stomach) cancer, germ cell tumor, head and neck cancer, leukemia, lip and oral cavity cancer, liver cancer, lung cancer, melanoma, Merkel cell carcinoma, neuroendocrine cancer, ovarian cancer, pancreatic cancer, penile cancer, pituitary tumor, prostate cancer, renal cancer, salivary gland cancers, sarcoma, skin cancer, small intestine cancer, squamous cell cancer, testicular cancer, throat cancer, thyroid cancer, urethral cancer, vaginal cancer, and post-transplant lymphoproliferative disorder (PTLD).

14. The method of claim 2, wherein the cancerous cell is of a cancer selected from the group consisting of breast cancer, colorectal cancer, melanoma, sarcoma, head and neck carcinoma, ovarian cancer, and prostate cancer.

15. The method of claim 2, wherein the cancerous cell is of breast cancer.

16. The method of claim 2, wherein the breast cancer is mixed breast cancer, wherein the mixed breast cancer comprises invasive and non-invasive breast cancer.

17. The method of claim 2, wherein the breast cancer is an invasive breast cancer.

18. The method of claim 2, wherein the breast cancer is a non-invasive breast cancer.

19. The method of claim 2, wherein the non-invasive breast cancer is ductal carcinoma in situ.

20. The method of claim 2, comprising administering 1 mg to 16 mg of SDM-25, or pharmaceutically acceptable salt thereof, to the subject.

21. The method of claim 2, comprising administering 8 mg of SDM-25, or pharmaceutically acceptable salt thereof, to the subject.

22.-112. (canceled)
